SEQUENCE LISTING (36 sequences)

SEQ ID NO: 1 - protein, 604 aa, synthetic construct
MYRMQLLSCIALSLALVTNSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGEGGGGSEEGGGGSEEGGGGSEEGGGGSEEGGGGSEEGGGGSEEGGGGSEEGGGGSEEGGGGS

SEQ ID NO: 2 - protein, 584 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGEGGGGSEEGGGGSEEGGGGSEEGGGGSEEGGGGSEEGGGGSEEGGGGSEEGGGGSEEGGGGS

SEQ ID NO: 3 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 57 aa, synthetic construct
SEEGGGGSEEGGGGSEEGGGGSEEGGGGSEEGGGGSEEGGGGSEEGGGGSEEGGGGS

SEQ ID NO: 7 - protein, 592 aa, synthetic construct
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARGGGSKDEL

SEQ ID NO: 8 - DNA, 1740 bp, synthetic construct
  source 1..1740: mol_type = other DNA
aaggacgtctacgtggtggaactggattggtacccagacgcccctggagaaatggtggtgctcacctgtgatacccccgaagaggacgggattacttggaccctggatcagtcatccgaagtgctgggatccggaaagaccctgacgatccaggtcaaggaattcggggacgcgggacagtacacttgccataagggcggcgaagtgctgtcccactccttgctcctgctccacaagaaagaagatggaatttggagcaccgacatcctgaaggaccagaaagagccgaagaacaagaccttcctgcgctgcgaggccaagaactacagcggacgctttacctgttggtggctgaccactatctccaccgacctgaccttctcggtcaagtcctcgcgcgggagcagcgatcctcagggagtgacttgcggtgccgcgactctgtcggccgaaagggtccggggcgacaacaaggagtacgagtactcagtcgagtgccaggaggacagcgcatgtccggccgctgaggaatccctgcctattgaagtcatggtggacgccgtccacaagctgaaatacgagaactacacctcctctttctttatccgggatatcatcaagcccgacccgcctaagaaccttcaactgaagccactgaagaacagcagacaagtggaggtgtcctgggagtacccggatacttggtccactccgcactcctatttctcgctcaccttctgcgtccaagtgcagggaaagtcgaaacgggagaagaaggacagagtgttcactgacaagaccagcgcaactgtgatctgtcggaagaacgcctccatttccgtgcgcgcccaggaccggtactattcctcatcctggtcggagtgggcctcagtgccctgcagcggcgggggcggttcgggtggtggcggcagcgggggcggagggtcccggaatctgccggtggcgacgcccgaccccggaatgttcccgtgcctgcaccatagccagaacttgctccgcgcggtgtctaacatgctgcagaaggcccggcagaccctggaattctacccctgcactagtgaagagatcgaccatgaagatattaccaaagacaagacttccaccgtggaagcgtgcctcccactggaactgaccaagaatgagtcctgcctgaattcaagggaaacttcgttcattaccaacggttcctgcctggcctcgagaaagacctccttcatgatggctctctgcctctcctcaatctacgaagatctcaaaatgtaccaagtggagttcaagaccatgaacgctaagctgctgatggacccgaagcggcagattttcctcgaccaaaacatgttggccgtgatcgacgagttgatgcaggcccttaacttcaactccgaaacagtgcctcagaagtcgagcctcgaggaacccgacttctacaagactaagatcaagctgtgcatcctgctgcacgcatttcgaattcgggccgtgaccatcgaccgcgtgatgagctacctgaacgcatccgggggcggcggggaaggaggaggcggctcagaggagggaggaggaggcagcgaggaaggaggcggaggaagcgaagaaggagggggaggctcagaagagggagggggtggatccgaggaaggcggaggaggatccgaagagggcggtggtggatcggaggaagggggaggcggttctgaggagggtggaggaggctcctga

SEQ ID NO: 9 - DNA, 1779 bp, synthetic construct
  source 1..1779: mol_type = other DNA
atgaagatgatgcttgttcgaagattccgggtcctgattctgatggtattcctcgtcgcatgcgccctgcacatcgccttggacctcctccctcggctggaacgcagaggagcacggccttccggagaaccgggctgctcgtgtgcacaaccagccgccgaagtcgctgcccctggatgggctcaagtcagaggacggcctggagaacccccggcagcttcctccgcggctggagatgcgggatggcccaacaagcacaccctccggatcctgcaggacttctcctccgacccctcctcgaatctgagctcccactccctcgaaaagctgcctcctgctgccgagccagctgaacgggccctgaggggtagagatcccggtgcactcaggccacatgaccctgcgcaccgacctctgttgagagatccgggtcctcggaggtctgaatcaccgccaggtcctggtggcgatgcgtcccttcttgcgcgcttgttcgagcacccgctgtaccgggtggcagtgcctcccctcaccgaggaagatgtgctgttcaacgtgaacagcgatacccgcctgagcccgaaagccgccgagaatcccgactggcctcatgccggagccgaaggagccgagttcctgagccctggagaagcggccgtggattcgtacccgaactggctaaagttccatatcggcatcaaccgctacgagctctactcgaggcataacccggccattgaggccctgctgcacgacctcagcagccaacggatcactagcgtggccatgaagtccgggggcactcagctgaagttgatcatgacctttcagaactacggccaggccctgtttaagcccatgaagcagacccgggagcaggaaacaccgccggactttttctacttctcggactacgaacgccacaacgccgagattgccgccttccacctggatcgcatcctggatttcagacgggtgccacctgtggcgggacgcatggtcaatatgaccaaagaaatccgcgacgtcactcgcgacaagaagctctggcggacgttcttcatttccccggccaacaacatctgcttctacggggagtgctcatactactgctccaccgagcatgccctttgcggaaagcccgaccagatcgaagggtccctggcagccttccttcccgacctgagcctggcgaagcgcaagacttggagaaacccatggcgccgctcataccacaagcggaagaaggcggagtgggaggtggaccccgactactgcgaggaagtcaagcagacccccccctacgactcctcccaccggattctggacgtgatggacatgactattttcgatttcctcatgggcaacatggaccggcaccactatgaaaccttcgaaaagttcgggaacgagactttcattattcacctggacaacggccggggattcggaaagtacagccacgacgaactctccatcctggtgccgctgcagcagtgttgccggatccgcaagtccacgtacctgagactgcagctgttggccaaagaggagtataagctgtcgctcctgatggccgaatccctccgcggcgatcaagtcgccccggtgctgtaccagccgcatctcgaggccctggacaggagactgcgcgtggtgctcaaagctgtgcgggattgtgtggaacggaacggcctgcattctgtggtggacgacgacctggacaccgaacacagagccgcatccgctaggggtggagggtccaaagatgagctgtga

SEQ ID NO: 10 - DNA, 1812 bp, synthetic construct
  source 1..1812: mol_type = other DNA
atgtatagaatgcagcttctgtcctgtatcgccctctccctcgcccttgtgaccaactccatctgggaactgaagaaggacgtctacgtggtggaactggattggtacccagacgcccctggagaaatggtggtgctcacctgtgatacccccgaagaggacgggattacttggaccctggatcagtcatccgaagtgctgggatccggaaagaccctgacgatccaggtcaaggaattcggggacgcgggacagtacacttgccataagggcggcgaagtgctgtcccactccttgctcctgctccacaagaaagaagatggaatttggagcaccgacatcctgaaggaccagaaagagccgaagaacaagaccttcctgcgctgcgaggccaagaactacagcggacgctttacctgttggtggctgaccactatctccaccgacctgaccttctcggtcaagtcctcgcgcgggagcagcgatcctcagggagtgacttgcggtgccgcgactctgtcggccgaaagggtccggggcgacaacaaggagtacgagtactcagtcgagtgccaggaggacagcgcatgtccggccgctgaggaatccctgcctattgaagtcatggtggacgccgtccacaagctgaaatacgagaactacacctcctctttctttatccgggatatcatcaagcccgacccgcctaagaaccttcaactgaagccactgaagaacagcagacaagtggaggtgtcctgggagtacccggatacttggtccactccgcactcctatttctcgctcaccttctgcgtccaagtgcagggaaagtcgaaacgggagaagaaggacagagtgttcactgacaagaccagcgcaactgtgatctgtcggaagaacgcctccatttccgtgcgcgcccaggaccggtactattcctcatcctggtcggagtgggcctcagtgccctgcagcggcgggggcggttcgggtggtggcggcagcgggggcggagggtcccggaatctgccggtggcgacgcccgaccccggaatgttcccgtgcctgcaccatagccagaacttgctccgcgcggtgtctaacatgctgcagaaggcccggcagaccctggaattctacccctgcactagtgaagagatcgaccatgaagatattaccaaagacaagacttccaccgtggaagcgtgcctcccactggaactgaccaagaatgagtcctgcctgaattcaagggaaacttcgttcattaccaacggttcctgcctggcctcgagaaagacctccttcatgatggctctctgcctctcctcaatctacgaagatctcaaaatgtaccaagtggagttcaagaccatgaacgctaagctgctgatggacccgaagcggcagattttcctcgaccaaaacatgttggccgtgatcgacgagttgatgcaggcccttaacttcaactccgaaacagtgcctcagaagtcgagcctcgaggaacccgacttctacaagactaagatcaagctgtgcatcctgctgcacgcatttcgaattcgggccgtgaccatcgaccgcgtgatgagctacctgaacgcatccgggggcggcggggaaggaggaggcggctcagaggagggaggaggaggcagcgaggaaggaggcggaggaagcgaagaaggagggggaggctcagaagagggagggggtggatccgaggaaggcggaggaggatccgaagagggcggtggtggatcggaggaagggggaggcggttctgaggagggtggaggaggctcc

SEQ ID NO: 11 - protein, 521 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 12 - protein, 519 aa, synthetic construct
IWELEKDVYVVELDWHPDAPGEMVVLTCHTPEEDDITWTSAQSSEVLGSGKTLTIQVKEFGDAGQYTCHKGGKVLSRSLLLIHKKEDGIWSTDILKEQKESKNKIFLKCEAKNYSGRFTCWWLTAISTDLKFSVKSSRGFSDPQGVTCGAVTLSAERVRVDNRDYKKYTVECQEGSACPSAEESLPIEVVVDAIHKLKYENYTSSFFIRDIIKPDPPTNLQLKPLKNSRHVEVSWEYPDTWSTPHSYFSLTFCVQAQGKNNREKKDRLCVDKTSAKVVCHKDAKIRVQARDRYYSSSWSDWASVSCSGGGGSGGGGSGGGGSRSLPTASPSPGIFQCLNHSQNLLRAVSNTLQKARQTLELYSCTSEEIDHEDITKDKTSTVEACLPLELTMNESCLASREISLITNGSCLASGKASFMTVLCLSSIYEDLKMYQMEFKAMNAKLLMDPKRQIFLDQNMLTAIDELLQALNFNSVTVPQKSSLEEPDFYKTKIKLCILLHAFRIRAVTIDRMMSYLNSS

SEQ ID NO: 13 - protein, 519 aa, synthetic construct
IWELEKNVYVVELDWHPDAPGEMVVLTCNTPEEDDITWTSDQSSEVLGSGKTLTIQVKEFADAGQYTCHKGGEVLSHSFLLIHKKEDGIWSTDILREQKESKNKIFLKCEAKNYSGRFTCWWLTAISTDLKFTVKSSRGSSDPQGVTCGAATLSAEKVRVDNRDYKKYTVECQEGSACPAAEESLPIEVVVDAIHKLKYENYTSSFFIRDIIKPDPPKNLQLKPLKNSRHVEVSWEYPDTWSTPHSYFSLTFGVQVQGKNNREKKDRLSVDKTSAKVVCHKDAKIRVQARDRYYSSSWSNWASVSCSGGGGSGGGGSGGGGSRNLPTPTPSPGMFQCLNHSQTLLRAISNTLQKARQTLEFYSCTSEEIDHEDITKDKTSTVEACLPLELTMNESCLASREISLITNGSCLASRKTSFMTTLCLSSIYEDLKMYQVEFKAMNAKLLMDPKRQIFLDQNMLTAIDELLQALNVNSVTVPQNSSLEEPDFYKTKIKLCILLHAFRIRAVTINRMMSYLNSS

SEQ ID NO: 14 - protein, 514 aa, synthetic construct
IWELEKNVYVVELDWYPNAPGEMVVLTCNTPEEDGITWTSDQSSEVLGTGKTLTIHVKEFGDAGQYTCRKGGAVLSQSLLLLHKKEDGIWSTDILKDQKEPKNKSFLKCEAKNYSGRFTCWWLTAISTDLKFSVKSSRGSTDPRGVTCGTATLSEDLGEYKKYRVECQEGSACPAAEESLPIEVVLEAVHKLKYENYTSSFFIRDIIKPDPPKNLQLNPLKNSRHVEISWEYPDTWSTPHSYFSLMFGVQVQGKNKREKKDKLFTDQISAKVTCHKDANIRVQARDRYYSSSWSEWASVSCNGGGGSGGGGSGGGGSRSLPATTAGPGMFKCLNHSQNLLKAVSNTLQKAKQTLEFYSCTSEEIDHEDITKDKTSTVEACLPLELATNESCLAARETSLITNGNCLTSGKTSFMTTLCLSSIYEDLKMYHVEFQAMNAKLLMDPKRQIFLDQNMLTAITELMQALNFNSETVPQKPSLEELDFYKTKIKLCILLHAFRIRAVTIDRMMSYLNSS

SEQ ID NO: 15 - protein, 519 aa, synthetic construct
IWELEKDVYVVELDWYPDAPGEMVVLTCNTPEEEGITWTSAQSNEVLGSGKTLTIQVKEFGDAGWYTCHKGGEVLSHSHLLLHKKEDGIWSTDILKDQKESKNKTFLKCEAKNYSGRFTCWWLTAISTDLKFSVKSSRGSSDPRGVTCGAATLSAERVSVDDREYKKYTVECQEGSACPAAEESLPIEIVVDAVHKLKYENYTSGFFIRDIIKPDPPKNLQLKPLKNSRQVEVSWEYPETWSTPHSYFSLTFSIQVQGKNKKERKDRLFMDETSATVTCHKDGQIRVQARDRYYSSSWSEWASVSCSGGGGSGGGGSGGGGSRNLPTATPGPGMFQCLNHSQNLLRTVSNTLQKARQTLEFYSCTSEEIDHEDITKDKSSTVAACLPLELAPNESCLASREISFITNGSCLTPGKASSMMTLCLSSIYEDLKMYQVEFKAMNAKLLIDPQRQIFLDENMLTAIDKLMQALNFNSETVPQKPSLEGLDFYKTKVKLCILLHAFRIRAVTINRMMGYLNAS

SEQ ID NO: 16 - protein, 8 aa, synthetic construct
  VARIANT 2: note = Any amino acid
  VARIANT 4: note = Any amino acid
  VARIANT 5: note = Any amino acid
SXQXXDEE

SEQ ID NO: 17 - protein, 4 aa, unidentified
SEEE

SEQ ID NO: 18 - protein, 4 aa, unidentified
SEEA

SEQ ID NO: 19 - protein, 4 aa, unidentified
SEEQ

SEQ ID NO: 20 - protein, 8 aa, synthetic construct
GGGSGGGG

SEQ ID NO: 21 - protein, 8 aa, synthetic construct
GGGEGGGG

SEQ ID NO: 22 - protein, 5 aa, synthetic construct
GGGGG

SEQ ID NO: 23 - protein, 4 aa, synthetic construct
GGGG

SEQ ID NO: 24 - protein, 5 aa, synthetic construct
GGGGS

SEQ ID NO: 25 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 26 - protein, 10 aa, synthetic construct
GGGGSGGGGS

SEQ ID NO: 27 - protein, 15 aa, synthetic construct
GGGGSGGGGSGGGGS

SEQ ID NO: 28 - protein, 20 aa, synthetic construct
GGGGSGGGGSGGGGSGGGGS

SEQ ID NO: 29 - protein, 25 aa, synthetic construct
GGGGSGGGGSGGGGSGGGGSGGGGS

SEQ ID NO: 30 - protein, 30 aa, synthetic construct
GGGGSGGGGSGGGGSGGGGSGGGGSGGGGS

SEQ ID NO: 31 - protein, 35 aa, synthetic construct
GGGGSGGGGSGGGGSGGGGSGGGGSGGGGSGGGGS

SEQ ID NO: 32 - protein, 40 aa, synthetic construct
GGGGSGGGGSGGGGSGGGGSGGGGSGGGGSGGGGSGGGGS

SEQ ID NO: 33 - protein, 45 aa, synthetic construct
GGGGSGGGGSGGGGSGGGGSGGGGSGGGGSGGGGSGGGGSGGGGS

SEQ ID NO: 34 - protein, 50 aa, synthetic construct; identical to SEQ ID NO: 25

SEQ ID NO: 35 - protein, 9 aa, synthetic construct
GGGGSGGGG

SEQ ID NO: 36 - protein, 9 aa, synthetic construct
GGGGEGGGG

(19) United States
(12) Patent Application Publication
(10) Pub. No.: US 2025/0339495 A1
(43) Pub. Date: Nov. 6, 2025

(54) FUSION POLYPEPTIDES AND FORMULATIONS THEREOF

(71) Applicant: Ankyra Therapeutics, Inc., Boston, MA (US)

(72) Inventors: Michael SCHMIDT, Wellesley, MA (US); Grigorios ZARBIS-PAPASTOITSIS, Watertown, MA (US)

(21) Appl. No.: 19/128,365

(22) PCT Filed: Nov. 8, 2023
(86) PCT No.: PCT/US2023/036996

§ 371 Date: May 8, 2025

Related U.S. Application Data
(60) Provisional application No. 63/424,013, filed on Nov. 9, 2022.

(51) Int. Cl. A61K 38/20; A61K 33/08; A61K 47/02; A61K 47/18; A61K 47/20; A61K 47/26

(52) U.S. Cl. CPC A61K 38/208; A61K 33/08; A61K 47/02; A61K 47/18; A61K 47/20; A61K 47/26

(57) ABSTRACT

The present disclosure provides surprisingly useful fusion polypeptides including an immunomodulatory moiety and a metal-hydroxide binding moiety, as well as various related technologies, including methods of making and of using such fusion polypeptides.

### CROSS-REFERENCE TO RELATED APPLICATIONS

The present application claims priority to and the benefit of U.S. Provisional Patent Application No. 63/424,013, filed on Nov. 9, 2022, the entirety of which is incorporated herein by reference.

### BACKGROUND

Useful fusion polypeptides have been developed in which a metal binding polypeptide is conjugated with an immunomodulatory domain (e.g., IL-12 immunomodulatory domain) (see, for example, published International Patent Application WO2020/263399). Particular uses of such fusion peptides include treatment of certain medical conditions, such as cancer.

### SUMMARY

The present disclosure provides particular compositions and formulations of a phosphorylated form of a fusion polypeptide, specifically including, for example, a phosphorylated form of an IL-12 fusion polypeptide (e.g., as described in published International Patent Application WO2020/263399). In some embodiments, provided technologies provides particularly stable compositions and formulations of a phosphorylated form of such fusion polypeptides.

Among other things, the present disclosure identifies the source of a problem with certain compositions or formulations comprising such phosphorylated fusion polypeptides, and provides solutions thereto. For example, the present disclosure proposes that relevant phosphorylated fusion polypeptides are unusually unstable in typical formulations and compositions, creating challenges for production and/or distribution, e.g., some standard formulations lead to agent deamidation, susceptibility to oxidation, and instabilities such as formation of visible particles triggered by shaking. Furthermore, without wishing to be bound by any particular theory, the present disclosure proposes that phosphorylated IL-12 fusion polypeptides may show unusual “stickiness” such that, in many standard formulations, agent sticks to surfaces of a vessel in which it is contained.

The present disclosure furthermore provides solutions to these problems and provides desirable compositions of provided phosphorylated fusion polypeptides and/or in some embodiments, formulations of phosphorylated fusion polypeptides complexed with a metal hydroxide. Among other things, the present disclosure provides metal-hydroxide-binding polypeptides, and fusion polypeptides that include them, which demonstrate high levels of adsorption to metal hydroxides.

Certain useful compositions comprise a phosphorylated fusion polypeptide that is or comprises a phosphorylated IL-12 fusion polypeptide in a Tris buffer formulation at pH around 6.5-8 (e.g., around 7.4). In some embodiments, compositions according to the present disclosure may also comprise addition of a salt (e.g., NaCl) and/or L-Methionine and/or sucrose and/or a surfactant (e.g., a Polysorbate). Without wishing to be bound by any particular theory L-Methionine may mitigate IL-12 polypeptide fusion susceptibility to oxidation and a surfactant (e.g., a Polysorbate) may mitigate formation of visible particles upon shaking the composition. Addition of a salt may stabilize the structure of the molecule via ionic interactions.

The present disclosure furthermore provides useful formulations comprising a fusion polypeptide metal-hydroxide complex comprising a phosphorylated fusion polypeptide (e.g., a phosphorylated IL-12 fusion polypeptide) in a Tris buffer formulation at pH around 6.5-8 (e.g., around 7.4). Formulations according to the present disclosure may also comprise addition of a salt and/or L-Methionine and/or a surfactant (e.g., a Polysorbate).

In some aspects, the present disclosure provides compositions comprising a phosphorylated form of a fusion polypeptide comprising: (a) an immunomodulatory polypeptide that comprises an interleukin-12 immune agonist moiety; and (b) a metal-hydroxide binding polypeptide, whose amino acid sequence includes a plurality of phosphorylation sites, so that the fusion polypeptide can adopt phosphorylated and unphosphorylated forms, Tris buffer, salt, sucrose, L-Methonine; and a surfactant, wherein the pH of the composition is within the range of about 6.5 and about 8. In some embodiments, phosphorylated fusion polypeptides, when exposed to a metal-hydroxide forms a complex therewith. In some embodiments, the metal hydroxide is aluminum hydroxide.

In some aspects, the present disclosure provides pharmaceutical formulations comprising a fusion polypeptide metal-hydroxide complex comprising a phosphorylated form of a fusion polypeptide comprising: (a) an immunomodulatory polypeptide that comprises an interleukin-12 immune agonist moiety; and (b) a metal-hydroxide binding polypeptide, whose amino acid sequence includes a plurality of phosphorylation sites, so that the fusion polypeptide can adopt phosphorylated and unphosphorylated forms, and a metabl hydroxide, Tris buffer, salt, sucrose, L-Methonine; and a surfactant, wherein the pH of the composition is within the range of about 6.5 and about 8.

In some aspects, the present disclosure provides methods for treating a subject, comprising administering a pharmaceutical composition according to the present disclosure.

In some aspects, the present disclosure provides methods of manufacturing a composition and/or a pharmaceutical formulation according to the present disclosure.

In some aspects, the present disclosure provides methods of characterizing a composition according to the present disclosure, by assessing the degree og phosphorylation of the fusion polypeptide.

### BRIEF DESCRIPTION OF THE DRAWINGS

FIG. 1 provides an exemplary schematic of fusion polypeptide metal-hydroxide complexes of the present disclosure. Fusion polypeptide metal-hydroxide complexes can be administered to a subject and result in enhanced retention and/or efficacy compared to an appropriate reference standard.

FIG. 2 provides a diagram of an exemplary fusion polypeptide of the present disclosure comprising a first (p40) and second (p35) IL12 immune agonist moieties and a metal-hydroxide binding polypeptide with a plurality of phosphorylation sites.

FIGS. 3A and 3B: show purity by size Exclusion Chromatography A) shows main peak (IL-12 fusion polypeptide) and B) shows high molecular weight species (HMW).

FIGS. 4A and 4B show purity by size Exclusion Chromatography A) shows main peak (IL-12 fusion polypeptide) and B) shows high molecular weight species (HMW).

FIG. 5 shows alum retention over time.

FIG. 6 shows IL-12 fusion polypeptide activity in when formulated in TBS or in an IL-12 fusion polypeptide composition conjugated to alum or without a metal hydroxide.

FIGS. 7A-E show quantification of ATP to determine PBMC viability—Donor 1. PBMCs were isolated from Donors 1 rested and stimulated with aqueous anti-CD3 (100 ng/mL) in the presence of pre-incubated IL-12 fusion polypeptides complexed with alum (ANK-101) (A-C) or without alum (IL-12-ABP) (D-E) at 12 different concentrations. Appropriate controls; negative control (unstimulated), positive control (soluble CD3 [5 μg/mL]+aqueous CD28 [2 μg/mL]) and vehicle control (formulation buffer [0.04%]). On Day 3 ATP was quantified using CellTiter-Glo® 2.0 Cell Viability Assay kit. Graphs show mean of triplicates ±SEM. Lower solid line: unstimulated; dotted line: vehicle; and Upper solid line: positive. ATP=adenosine triphosphate; CD=cluster of differentiation; Conc=concentration; PBMC=peripheral blood mononuclear cell; SEM=standard error of the mean. ANK-101: IL-12 fusion polypeptide complexed to alum. IL-12-ABP: IL-12 fusion polypeptide.

FIGS. 8A-E show quantification of ATP to determine PBMC viability—Donor 2. PBMCs were isolated from Donor 2 rested and stimulated with aqueous anti-CD3 (100 ng/mL) in the presence of pre-incubated IL-12 fusion polypeptides complexed with alum (ANK-101) (A-C) or without alum (IL-12-ABP) (D-E) at 12 different concentrations. Appropriate controls; negative control (unstimulated), positive control (soluble CD3 [5 μg/mL]+aqueous CD28 [2 μg/mL]) and vehicle control (formulation buffer [0.04%]). On Day 3 ATP was quantified using CellTiter-Glo® 2.0 Cell Viability Assay kit. Graphs show mean of triplicates ±SEM. Lower solid line: unstimulated; dotted line: vehicle; and Upper solid line: positive. ATP=adenosine triphosphate; CD=cluster of differentiation; Conc=concentration; PBMC=peripheral blood mononuclear cell; SEM=standard error of the mean. ANK-101: IL-12 fusion polypeptide complexed to alum. IL-12-ABP: IL-12 fusion polypeptide.

FIGS. 9A-E show accumulation of IFNγ in PBMC Cultures—Donor 1. PBMCs were isolated from Donor 1 rested and stimulated with aqueous anti-CD3 (100 ng/mL) in the presence of pre-incubated IL-12 fusion complexed with alum (ANK-101) (A-C) or without alum (IL-12-ABP) (D-E) polypeptide at 12 different concentrations. Appropriate controls; negative control (unstimulated), positive control (soluble CD3 [5 μg/mL]+aqueous CD28 [2 μg/mL]) and vehicle control (formulation buffer [0.04%]). On Day 3 cell culture supernatants were harvested and analysed by TR-FRET. Graphs show mean of triplicates ±SEM. Lower solid line: unstimulated; dotted line: vehicle; and Upper solid line: positive. CD=cluster of differentiation; Conc=concentration; EC50=half-maximal effective concentration; IFNγ=interferon gamma; NA=not applicable; PBMC=peripheral blood mononuclear cell; SEM=standard error of the mean; TR-FRET=time-resolved fluorescence energy transfer. ANK-101: IL-12 fusion polypeptide complexed to alum. IL-12-ABP: IL-12 fusion polypeptide.

FIGS. 10A-E show accumulation of IFNγ in PBMC Cultures—Donor 2. PBMCs were isolated from Donor 2 rested and stimulated with aqueous anti-CD3 (100 ng/mL) in the presence of pre-incubated IL-12 fusion polypeptide complexed with alum (ANK-101) (A-C) or without alum (IL-12-ABP) (D-E) at 12 different concentrations. Appropriate controls; negative control (unstimulated), positive control (soluble CD3 [5 μg/mL]+aqueous CD28 [2 μg/mL]) and vehicle control (formulation buffer [0.04%]). On Day 3 cell culture supernatants were harvested and analysed by TR-FRET. Graphs show mean of triplicates ±SEM. Lower solid line: unstimulated; dotted line: vehicle; and Upper solid line: positive. CD=cluster of differentiation; Conc=concentration; EC50=half-maximal effective concentration; IFNγ=interferon gamma; NA=not applicable; PBMC=peripheral blood mononuclear cell; SEM=standard error of the mean; TR-FRET=time-resolved fluorescence energy transfer.

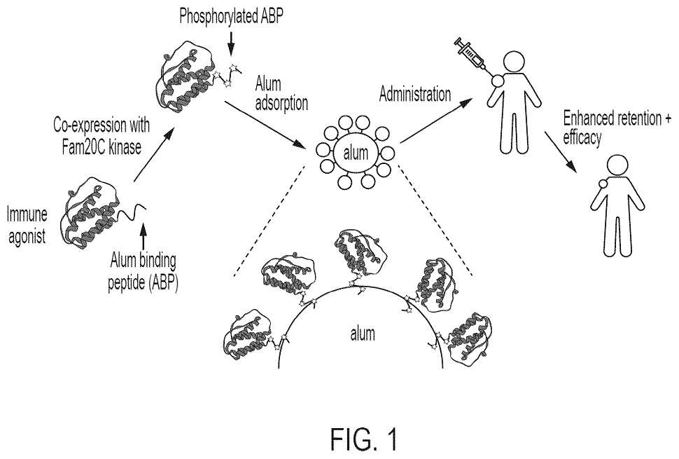

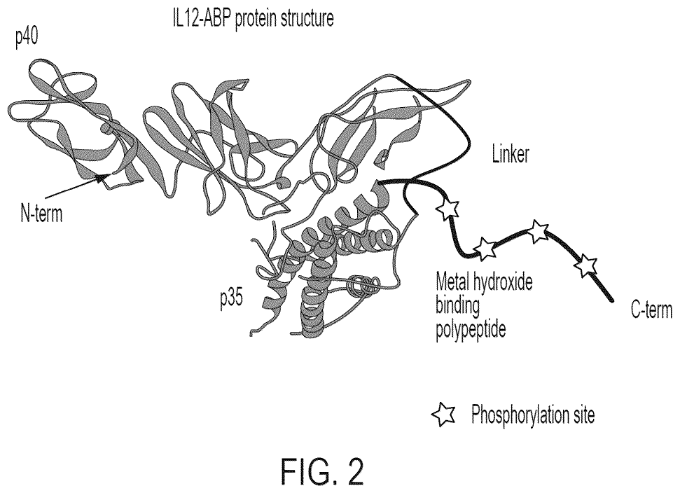

### DEFINITIONS

Administration: As used herein, the term “administration” typically refers to application of a composition to a subject or system. Those of ordinary skill in the art will be aware of a variety of routes that may, in appropriate circumstances, be utilized for administration to a subject, for example a human. For example, in some embodiments, administration may be systemic; in some embodiments, administration may be local. In some embodiments, administration may be enteral; in some embodiments, administration may be parenteral. In some embodiments, administration may be by injection (e.g., intramuscular, intratumoral, intravenous, or subcutaneous injection). In some embodiments, injection may involve bolus injection, drip, perfusion, or infusion. In many embodiments, administration in accordance with the present disclosure is by intratumoral injection.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them.

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class such as, for example, small molecules, polypeptides, nucleic acids, carbohydrates, lipids, metals, and/or any other entity that shows the relevant activating activity. In some embodiments, an agonist may be direct (in which case it exerts its influence directly upon its target, for example by physically binding to such target); in some embodiments, an agonist may be indirect (in which case it exerts its influence by other than binding to its target; e.g., by interacting with a regulator of the target, so that level and/or activity of the target is altered).

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein, the term “animal” refers to a member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a horse, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently, electrostatically, or otherwise associated with a carrier entity and/or in a biological system or cell). Binding between two entities may be considered “specific” if, under the conditions assessed, the relevant entities are more likely to associate with one another than with other available binding partners.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be one described as utilized in an antibody-drug conjugate as described or discussed in one or more of Govindan et al, The Scientific World JOURNAL 10:2070, 2010, -2089). In some embodiments, a chemotherapeutic agent may be or comprise one or more of farnesyl-thiosalicylic acid (FTS), 4-(4-Chloro-2-methylphenoxy)-N-hydroxybutanamide (CMH), estradiol (E2), tetramethoxystilbene (TMS), 6-tocatrienol, salinomycin, or curcumin Combination Therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Epitope: as used herein, the term “epitope” refers to a moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Functional: As used herein, the term “functional” is used to refer to a form or fragment of an entity that exhibits a particular property and/or activity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Genome: As used herein, the term “genome” refers to the total genetic information carried by an individual organism or cell, represented by the complete DNA sequences of its chromosomes.

Host cell: as used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO KI, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Host Cell Protein(s)” or HCP(s): As used herein, refers to proteins that may be present in a cell extract or preparation, for example because they were produced by or otherwise contained in or on a host cell in which a fusion polypeptide (e.g., a phosphorylated or unphosphorylated fusion polypeptide) as described herein is produced, and that are not the fusion polypeptide. In some embodiments, provided technologies (e.g., provided manufacturing methods, such as provided purification methods) exclude or reduce HCPs from preparation(s) of the fusion polypeptide (e.g., from preparations of phosphorylated fusion polypeptide as described herein). A “reduced HCP preparation” describes a preparation that contains reduced HCPs relative, for example, to that amount present before application of a relevant purification step (e.g., as provided herein) and/or relative to that achieved through a different purification technology. In some embodiments, provided technologies achieve production of fusion polypeptide preparations (e.g., preparations of phosphorylated fusion polypeptide) in which HCP are undetectable, for example, using e.g., an ELISA method. In some embodiments, removal of HCP may be monitored or assessed, for example, during or after purification of a fusion polypeptide (e.g., a phosphorylated form thereof) as described herein, for example from a host cell which may, in some embodiments, be an engineered mammalian cell as described herein (e.g., that expresses the fusion polypeptide and a kinase that phosphorylates it at a ratio within a range of about 4:1 to 10:1, for example at a ratio of about 8:1.

“Improved,” “Increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Linker: as used herein, is used to refer to that portion of a multi-element agent that connects different elements to one another. For example, those of ordinary skill in the art appreciate that a polypeptide whose structure includes two or more functional or organizational moieties or domains often includes a stretch of amino acids between such moieties or domains that links them to one another. In some embodiments, a polypeptide comprising a linker element has an overall structure of the general form S1-L-S2, wherein S1 and 52 may be the same or different and represent two moieties or domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger et al., Proc. Natl. Acad. Sci. USA 90:6444, 1993; Poljak et al. Structure 2:1121, 1994).

Modulator: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level

Moiety Those skilled in the art will appreciate that a “moiety” is a defined chemical group or entity with a particular structure and/or or activity, as described herein. Typically, a “moiety” is part of, less than the entirety of, a molecule or entity.

Mutant: As used herein, the term “mutant” refers to an entity that shows significant structural identity with a reference entity but differs structurally from the reference entity in the presence or level of one or more chemical moieties as compared with the reference entity. In many embodiments, a mutant also differs functionally from its reference entity. In general, whether a particular entity is properly considered to be a “mutant” of a reference entity is based on its degree of structural identity with the reference entity. As will be appreciated by those skilled in the art, any biological or chemical reference entity has certain characteristic structural elements. A mutant, by definition, is a distinct chemical entity that shares one or more such characteristic structural elements. To give but a few examples, a small molecule may have a characteristic core structural element (e.g., a macrocycle core) and/or one or more characteristic pendent moieties so that a mutant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a mutant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a mutant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a mutant polypeptide does not share at least one characteristic sequence element with a reference polypeptide. In some embodiments, the reference polypeptide has one or more biological activities. In some embodiments, a mutant polypeptide shares one or more of the biological activities of the reference polypeptide. In some embodiments, a mutant polypeptide lacks one or more of the biological activities of the reference polypeptide. In some embodiments, a mutant polypeptide shows a reduced level of one or more biological activities as compared with the reference polypeptide.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for a particular route of administration, e.g., as described herein.

Pharmaceutically acceptable: As used herein, the phrase “pharmaceutically acceptable” is used to refer to an agent or entity that, within the scope of sound medical judgment, is suitable for use in contact with tissues of human beings and/or animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Predetermined: By predetermined is meant deliberately selected, for example as opposed to randomly occurring or achieved.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference standard: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Viral inactivation or removal: As used herein, the term “viral inactivation or removal” describes inactivation or removal of a virus that may be contained in a sample such as, for example, a cell extract or a fusion polypeptide preparation. In some embodiments, a virus present in a sample may have originated from a source material (e.g. a host cell); alternatively or additionally, in some embodiments, a virus present in a sample may have been introduced, e.g., during processing of such source material. Those skilled in the art will be aware of a variety of technologies for accomplishing viral inactivation or removal such as, for example, by, pH inactivation, chemical inactivation (e.g., through use of a chemical agent such as a surfactant), etc. Those skilled in the art will appreciate that “pH viral inactivation” involves comprises exposing a virus (e.g., a sample containing a virus) to a pH that inactivates (e.g., has been established to inactivate) the virus.

Wild-type: As used herein, the term “wild-type” has its art-understood meaning and refers to a form of an entity (e.g., a polypeptide or nucleic acid) that has a structure and/or activity as found in nature in a “normal” (as contrasted with mutant, diseased, altered) state or context. In some embodiments, more than one “wild type” form of a particular polypeptide or nucleic acid may exist in nature, for example as “alleles” of a particular gene or normal variants of a particular polypeptide.. In some embodiments, that form (or those forms) of a particular polypeptide or nucleic acid that is most commonly observed in a population (e.g., in a human population) is the “wild type” form.

### DETAILED DESCRIPTION OF CERTAIN EMBODIMENTS

### Fusion Polypeptides

In some embodiments, fusion polypeptides according to the present disclosure can adopt phosphorylated and unphosphorylated forms.

### Immunomodulatory Polypeptide

Fusion polypeptides of the present disclosure comprise at least one immunomodulatory polypeptide.

In some embodiments, an immunomodulatory polypeptide (e.g., an immune agonist moiety) activates or inhibits activity of a cell of the immune system (e.g., is signaling competent). In some embodiments, an immunomodulatory polypeptide (e.g., an immune agonist moiety) is assessed, for example as part of a fusion polypeptide, e.g., as described herein.

For example, in some embodiments, signal competency is characterized in that, when assessed for binding to a particular binding partner, an immune agonist moiety or moieties or functional fragments thereof displays binding comparable to that of a reference standard (e.g., a wild-type polypeptide). For example, in some embodiments, signal competency is characterized in that, when assessed for a biological effect, e.g., in vitro or in vivo, an immune agonist moiety or moieties or functional fragments thereof displays said biological effect comparable to that of a reference standard (e.g., a wild-type polypeptide).

In some embodiments, an immunomodulatory polypeptide comprises an interleukin-12 (IL-12) immunomodulatory polypeptide (e.g., an IL-12 immune agonist moiety).

IL-12 is a pro-inflammatory cytokine that plays an important role in innate and adaptive immunity. Wild type IL-12 is a heterodimeric protein comprising two subunits, p35 (IL-12A; GenBenk GeneID: 3592) and p40 (IL-12B; GenBank GeneID: 3593), connected by disulfide bonds. Binding of IL-12 to the IL-12 receptor complex (IL-12Rβ1/IL-12Rβ2) on T cells and Natural Killer (NK) cells leads to signaling via signal transducer and activator of transcription 4 (STAT4) and subsequent interferon γ (IFN-γ) production and secretion.

IL-12 subunits, IL-12A and IL-12B, can also form heterodimers with other IL-12 family members. For example, IL-12A may also dimerize with Epstein-Barr virus induced gene 3 (EBI3) to form IL-12 family member, IL-35 and IL-12B may dimerize with a p19 monomer, to form IL-12 family member, IL23.

IL-12 plays important roles in the innate and adaptive immune response, and dysregulation of IL-12 has been implicated in a number of disease states. Exemplary such disease states include, but are not limited to, inflammatory bowel disease, psoriasis, diabetes mellitus, multiple sclerosis, rheumatoid arthritis, cancer, lupus erythematosus, primarily biliary cholangitis and Sjögren's syndrome (Ullrich et al. EXCLI journal vol. 19 1563-1589. 11 Dec. 2020). Use of IL-12 as a therapeutic modality has been studied extensively, including for treatment of tumors (Nastala C L et al. J Immunol. 1994 Aug. 15; Lasek et al. Cancer immunology, immunotherapy: CII vol. 63,5 (2014): 419-35).

In some embodiments, an immunomodulatory polypeptide disclosed herein is or comprises an IL-12 immune agonist moiety. In some embodiments, an immunomodulatory polypeptide disclosed herein comprises a plurality of IL-12 immune agonist moieties. In some embodiments, an immunomodulatory polypeptide disclosed herein comprises exactly two IL-12 immune agonist moieties. In some embodiments, two or more IL-12 moieties of a plurality of (e.g., two) IL-12 moieties are the same moiety. In some such embodiments, a plurality (e.g., two) IL-12 moieties are different moieties. In some such embodiments, an IL-12 moiety comprises an IL-12A polypeptide or functional fragment thereof. In some embodiments, an IL-12 moiety comprises an IL-12B polypeptide or functional fragment thereof.

In some embodiments, an IL-12B immune agonist moiety is located N-terminal to an IL-12A immune agonist moiety in an immunomodulatory polypeptide. In some embodiments, an IL-12A immune agonist moiety is located N-terminal to an IL-12B immune agonist moiety in an immunomodulatory polypeptide.

In some embodiments, an immunomodulatory polypeptide comprising a plurality (e.g., two) IL-12 moieties (e.g., IL-12A and/or IL-12B) are linked directly. In some embodiments, an immunomodulatory polypeptide comprising a plurality (e.g., two) IL-12 moieties (e.g., IL-12A and/or IL-12B) are linked via a first linker. Non-limiting examples of linkers are discussed elsewhere herein.

In some embodiments, an immunomodulatory polypeptide disclosed herein comprises an IL-12A and/or IL-12B immune agonist moiety comprising a variant. In some embodiments, an IL-12A and/or IL-12B immune agonist moiety variant comprises a substitution, deletion, addition, and/or insertion of relative to a wild-type IL-12A or IL-12B polynucleotide or amino acid sequence. In some embodiments, an IL-12A and/or IL-12B immune agonist moiety comprises a plurality of variants. In some embodiments, a plurality of variants comprises one or more of a substitution, deletion, addition, and/or insertion relative to a wild-type IL-12A or IL-12B. In some embodiments, a variant comprises a substitute that does not change the amino acid sequence relative to a wild-type IL-12A or IL-12B.

In some embodiments, an immunomodulatory polypeptide disclosed herein comprises an IL-12A and/or IL-12B immune agonist moiety that is a functional fragment thereof (e.g., a signaling competent fragment). In some embodiments, an immunomodulatory polypeptide comprises a functional IL-12A fragment. In some embodiments, an immunomodulatory polypeptide comprises a functional IL-12B fragment. In some embodiments, an immunomodulatory polypeptide comprises a full length IL-12A and a functional IL-12B fragment. In some embodiments, an immunomodulatory polypeptide comprises a full length IL-12B and a functional IL-12A fragment.

In some embodiments, a IL-12A or IL-12B fragment comprises or consists of at least 5%, 10%, 15%, 20%, 25%, 30%, 35%, 40%, 45%, 50%, 55%, 60%, 65%, 70%, 75%, 80%, 85%, 90%, 95%, 99% or more of the monomeric units (e.g., residues) as found in wild-type IL-12A or IL-12B.

In some embodiments, an immunomodulatory polypeptide disclosed herein comprises an IL-12A and/or IL-12B immune agonist moiety that is a human IL-12A and/or IL-12B immune agonist moiety.

In some embodiments, an immunomodulatory polypeptide disclosed herein comprises an IL-12B immune agonist moiety having at least 80% sequence identity to SEQ ID NO: 3, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity to SEQ ID NO: 3. In some embodiments, an immunomodulatory polypeptide disclosed herein comprises an IL-12A immune agonist moiety having at least 80% sequence identity to SEQ ID NO: 4, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity to SEQ ID NO: 4. In some embodiments, an immunomodulatory polypeptide disclosed herein comprises an IL-12B immune agonist moiety having at least 80% sequence identity to SEQ ID NO: 3 and an IL-12A immune agonist moiety having at least 80% sequence identity to SEQ ID NO: 4.

In some embodiments, an immunomodulatory polypeptide disclosed herein comprises an amino acid sequence having at least 80% sequence identity to SEQ ID NO: 5, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity to SEQ ID NO: 5.

Without being bound to a particular theory, a hydroxyl replacement (e.g., with phosphate groups) can increase a polypeptide's adsorption via ligand exchange with a metal hydroxide (e.g., aluminum hydroxide), and furthermore can improve tumor retention and anti-tumor efficacy of such polypeptide (e.g., specifically of a fusion polypeptide comprising an immunomodulatory polypeptide and a metal-hydroxide-binding polypeptide in which such hydroxyl replacement has occurred.

In some embodiments, immunomodulatory polypeptides according to the present invention can adopt phosphorylated and unphosphorylated forms. In some embodiments, an immunomodulatory polypeptide comprises at least one amino acid that can be phosphorylated. In some embodiments, an immunomodulatory polypeptide comprises at least one kinase targets motif. In some embodiments, an immunomodulatory polypeptide does not comprise a kinase targets motif. In those embodiments, the immunomodulatory polypeptide may still comprise an amino acid that can be phosphorylated. In some embodiments, an immunomodulatory polypeptide comprising one or more phosphorylated amino acids contributes to metal strong binding to metal hydroxide, e.g., aluminum hydroxide. Table 1 show exemplary serine residues that may be phosphorylated in the immunomodulatory domain (e.g., S43, S154, S, 168, S233, S365, S398, S481 of SEQ ID NO. 2). In some embodiments, an immunomodulatory polypeptide comprises at least one phosphorylated serine. In some embodiments, an immunomodulatory polypeptide comprises at least two phosphorylated serine residues. In some embodiments, an immunomodulatory polypeptide comprises at least three phosphorylated serine residues. In some embodiments, an immunomodulatory polypeptide comprises at least four phosphorylated serine residues. In some embodiments, an immunomodulatory polypeptide comprises at least five phosphorylated serine residues, such as six serine residues, such as seven serine residues, such as eight serine residues, such as nine serine residues, such as ten serine residues.

### Metal-Hydroxide Binding Polypeptide

In some embodiments, fusion polypeptides of the present disclosure comprise at least one metal-binding polypeptide.

The present disclosure provides metal-hydroxide-binding polypeptides, and fusion polypeptides that include them, which demonstrate high levels of adsorption to metal hydroxides and also desirable manufacturing characteristics (e.g., one or more of reproducibility, consistency, production of a homogeneously-phosphorylated fusion polypeptide, etc.).

In some embodiments, a fusion polypeptide comprises two or more metal-binding polypeptides (e.g., two or more alum-binding polypeptides). In some embodiments, a fusion polypeptide comprises two or more metal-binding polypeptides that are the same; in some such embodiments, all metal-binding polypeptides in a fusion polypeptide in accordance with the present disclosure are the same. In some such embodiments, a fusion polypeptide comprises two or more metal-binding polypeptides that at are different from one another.

As discussed above, a metal-binding peptide can be fused to an immunomodulatory polypeptide allowing for strong binding to a metal hydroxide, such as aluminum hydroxide. Various immunomodulatory polypeptides can be fused to metal-binding peptides. Without being bound to a particular theory, metal-binding polypeptides adsorbed to alum in serum can be used to retain proteins and peptides in tumors.

In some embodiments, a metal-hydroxide binding polypeptide comprises an amino acid sequence that includes a plurality of phosphorylation sites, so that it can adopt phosphorylated and unphosphorylated forms. In some embodiments, a metal-hydroxide binding polypeptide comprises at least one kinase target motif. A target kinase motif comprises an amino acid that is phosphorylated by a kinase. Amino acids that are typically phosphorylated include a hydroxyl, such as serine (Ser, S), threonine (Thr, T), and tyrosine (Tyr, Y) residues. A kinase motif refers to the amino acid sequence immediately N- and/or C-terminal to the amino acid residue capable of being phosphorylated. Without wishing to be bound by any one theory, many kinases comprise structural features that confer specificity such that the kinase phosphorylates a particular amino acid (e.g., serine, threonine, or tyrosine) of a particular kinase target motif.

Kinase target motifs recognized are highly diverse depending on the particular type of kinase. In some embodiments, the present disclosure provides metal-hydroxide binding polypeptides comprising one or more kinase target motifs of a secretory pathway kinase. The secretory pathway, which is the pathway by which a cell secretes proteins and/or other biomolecules into the extracellular space, refers to the endoplasmic reticulum (ER), Golgi apparatus (Golgi), cell membrane, and lysosomal storage compartments as well as the vesicles that travel between them. Secretory pathway kinases are localized throughout the secretory pathway (e.g., at the E R, Golgi, etc.) and function to phosphorylate proteins destined for secretion (Sreelatha et al. Biochimica et biophysica acta vol. 1854,10 Pt B (2015): 1687-93).

In some embodiments, a relevant kinase is a naturally occurring secretory pathway kinase (e.g., is endogenously targeted to the secretory pathway to function). In some embodiments, a secretory pathway kinase comprises a signal sequence that targets the kinase to the secretory pathway. Naturally-occurring human secretory pathway kinases include, for example, four-jointed box kinase 1, Fam20A, Fam20B, Fam20C, vertebrate lonesome kinase (VLK), SGK196, and Fam69A, Fam69B, and Fam69C.

In some embodiments, a relevant kinase is a non-naturally occurring secretory pathway kinase. In some embodiments, a non-naturally occurring kinase is produced by linking a secretory signal peptide to a kinase endogenously localized to a non-secretory pathway cellular compartment.

In some embodiments, a kinase target motif is a target kinase motif of a secretory pathway kinase. In some embodiments, a secretory pathway kinase target kinase motif comprises an S-X-E motif. For example, Fam20C phosphorylates serine and has been shown to phosphorylate kinase targets motif comprising the amino acid sequence Ser-X-Glu (e.g., S-X-E), Ser-X-pSer (e.g., S-X-pS), and Ser-X-Gln-X-X-Asp-Glu-Glu (S-X-Q-X-X-D-E-E) wherein X is any amino acid, and p5 is phosphorylated serine (Mercier, et al (1981) Biochimie, 63: 1-17; Mercier et al (1971) Eur J. Biochem. 23:41-51; Lasa-Benito (1996) FEES Lett. 382:149; Brunati, et al (2000) 3:765, Tagliabracci, et al (2015) Cell 161:1619-1632; Tagliabracci, et al (2012) Science 336:1150-1153). In some embodiments, a target kinase motif comprises the amino acid sequence SEEE. In some embodiments, a target kinase motif comprises the amino acid sequence SEEA. In some embodiments, a target kinase motif comprises the amino acid sequence SEEQ. In some embodiments, a target kinase motif comprises the amino acid sequence SEE.

In some embodiments, a metal-hydroxide binding polypeptide comprises at least one, two, three, four, five, six, seven, eight, nine, ten, eleven, or twelve target kinase motifs. In some embodiments, a metal-hydroxide binding polypeptide comprises at least one, two, three, four, five, six, seven, eight, nine, ten, eleven, or twelve S-X-E motifs. In some embodiments, a metal-hydroxide binding polypeptide comprises more than four S-X-E motifs. In some embodiments, a metal-hydroxide binding polypeptide comprises eight S-X-E motifs In some embodiments, the number of target kinase motifs (e.g., S-X-E motifs) contributes to the number of phosphorylated residues on a metal-hydroxide binding polypeptide.. In some embodiments, a metal-hydroxide binding polypeptide comprises eight S-E-E motifs.

In some embodiments, a metal-hydroxide binding polypeptide is a metal-hydroxide binding polypeptide whose amino acid sequence includes a plurality of phosphorylation sites. In some embodiments, a plurality of phosphorylation sites comprises at least one, two, three, four, five, six, seven, eight, nine, ten, eleven, or twelve target kinase motifs. In some embodiments, a plurality of phosphorylation sites comprises at least one, two, three, four, five, six, seven, eight, nine, ten, eleven, or twelve S-X-E motifs. In some embodiments, a plurality of phosphorylation sites comprises more than four S-X-E motifs. In some embodiments, a plurality of phosphorylation sites comprises more than eight S-X-E motifs. In some embodiments, the number of target kinase motifs (e.g., S-X-E motifs) contributes to the number of phosphorylated residues on a metal-hydroxide binding polypeptide.

In some embodiments, the at least one, two, three, four, five, six, seven, eight, nine, ten, eleven, or twelve target kinase motifs (e.g., S-X-E motifs) are directly adjacent (e.g., linked) to the next target kinase (e.g., S-X-E motif). In some embodiments, the at least one, two, three, four, five, six, seven, eight, nine, ten, eleven, or twelve target kinase motifs (e.g., S-X-E motifs) are separated (e.g., linked) to the next target kinase motif (e.g., S-X-E motif) by a spacer. In some embodiments, the spacer comprises at least one glycine residue. In some embodiments, the spacer comprises a plurality of glycine residues. In some embodiments, the spacer comprises three glycine residues. In some embodiments, the spacer comprises at least four glycine residues. In some embodiments, the spacer has a sequence that comprises four glycine residues. In some embodiments, the spacer has an amino acid sequence comprising GGGSGGGG. In some embodiments, the spacer has an amino acid sequence comprising GGGEGGGG. In some embodiments, the spacer has an amino acid sequence comprising GGGGG. In some embodiments, the spacer has an amino acid sequence comprising GGGG.

In some embodiments, a metal-hydroxide binding polypeptide comprises four S-X-E motifs and three spacers comprising four glycine residues. In some embodiments, a metal-hydroxide binding polypeptide comprises six S-X-E motifs and five spacers comprising four glycine residues. In some embodiments, a metal-hydroxide binding polypeptide comprises eight S-X-E motifs and seven spacers comprising four glycine residues. In some embodiments, a metal-hydroxide binding polypeptide comprises eight S-X-E motifs and eight spacers comprising four glycine residues. In some embodiments, a metal-hydroxide binding polypeptide comprises eight motifs with the amino acid sequence, SEE, and eight spacers comprising four glycine residues.

In some embodiments, a metal-hydroxide binding polypeptide comprise an ending sequence (e.g., an amino acid sequence) at the c-terminus of the fusion polypeptide. In some embodiments, an ending sequence comprises a plurality of amino acid residues. In some embodiments, a plurality of amino acid residues comprises GGGG. In some such embodiments, an ending sequence comprises the amino acid sequence GGGGS.

In some embodiments, a desired (e.g., optimal) number of kinase target motifs and/or spacing of kinase motifs may be determined based on one or more of, for example, desired phosphate content to achieve strong metal-hydroxide retention and/or avoidance of one or more manufacturing challenges (e.g., which the present disclosure appreciates may be associated with highly phosphorylated elements). In some embodiments, a desired (e.g., optimal) number and/or spacing of kinase motifs results in an exposure of the polypeptide to the kinase to achieve a desired level of fusion polypeptide phosphorylation. In some embodiments, improved fusion polypeptides as described herein, results in one or more of improved reproducibility, consistency, and/or production of a homogenously-phosphorylated fusion polypeptide. For example, in some embodiments, provided technologies achieve reproducible manufacturing of comparable preparations (e.g., preparations that are consistently within established parameters) of fusion polypeptides (e.g., phosphorylated fusion polypeptides) and/or complexes as described herein. For example, in some embodiments, provided technologies achieve reduced immunogenicity compared to an appropriate reference standard.

In some embodiments, degree of phosphorylation (e.g., the average number of phosphate molecules per polypeptide) is 0.5-7, 1-7, 2-7, 3-7, 4-7, 5-7, 6-7, 0.5-6, 0.5-5, 0.5-4, 1-6, 2-6, 3-6, 4-6, 5-6, 7-8, 8-9, 9-10, 10-11, 11-12 or 13-14. In some embodiments, degree of phosphorylation (e.g., the average number of phosphate molecules per polypeptide) is 3, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 7.10, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, or 12.

### Linkers

In some embodiments, fusion polypeptides as described herein may include one or more linkers or spacers.

For example, in some embodiments, fusion polypeptides comprise an immunomodulatory polypeptide comprising a first and a second immune agonist moiety. In some embodiments, a first and a second immune agonist moiety are linked via a first linker.

In some embodiments, fusion polypeptides of the present disclosure comprise an immunomodulatory polypeptide and a metal-hydroxide binding polypeptide. In some embodiments, an immunomodulatory polypeptide and a metal-hydroxide binding polypeptide are linked via a second linker.

In some embodiments, a first linker and/or a second linker is a polypeptide linker. In some embodiments, a polypeptide linker is synthetic. For example, a synthetic polypeptide linker may comprise non-naturally occurring polypeptides which are modified forms of naturally occurring polypeptides.

In some embodiments, polypeptide linkers of the present disclosure are at least one amino acid in length and can be any suitable number of amino acids. In some embodiments, a polypeptide linker is 1, 2, 3, 4, 5, 6, 7, 8, 9, 10, 11, 12, 13, 14, 15, 16, 17, 18, 19, 20, 25, 30, 35,40, 45, or 50 amino acids in length.

In some embodiments, a first linker comprises a polypeptide linker. In some embodiments, a first linker comprises or consists of a Glycine-Serine (Gly-Ser or G-S linker). A Gly-Ser linker is a polypeptide linker that consists of glycine and serine residues. In some embodiments, a Gly-Ser linker comprises an amino acid sequence of (Gly₄Ser)ₙ, wherein n is a positive integer (e.g., 1, 2, 3, 4, 5, 6, 7, 8, 9, or 10). In some embodiments, a Gly-Ser linker is (Gly₄Ser)₁. In some embodiments, a Gly-Ser linker is (Gly₄Ser)₂. In some embodiments, a Gly-Ser linker is (Gly₄Ser)₃. In some embodiments, a Gly-Ser linker is (Gly₄Ser)₄. In some embodiments, a Gly-Ser linker is (Gly₄Ser)₅. In some embodiments, a Gly-Ser linker is (Gly₄Ser)₆. In some embodiments, a Gly-Ser linker is (Gly₄Ser)₇. In some embodiments, a Gly-Ser linker is (Gly₄Ser)₈. In some embodiments, a Gly-Ser linker is (Gly₄Ser)₉. In some embodiments, a Gly-Ser linker is (Gly₄Ser)₁₀.

In some embodiments, a second linker comprises a polypeptide linker. In some embodiments, a second linker comprises a plurality of glycine residues. In some embodiments, a second linker comprises a polypeptide linker with the amino acid sequence, GGGGSGGGG. In some embodiments, a second linker comprises a polypeptide linker with the amino acid sequence, GGGGEGGGG.

### Variants

In some embodiments, an immunomodulatory polypeptide or a metal-hydroxide-binding polypeptide utilized in accordance with the present disclosure is a variant of a relevant reference polypeptide (e.g., a wild type polypeptide or functional portion thereof).

In some embodiments, a variant shows at least 70% identity to its reference polypeptide. In some such embodiments, a variant shows at least 70%, 75%, 80%, 85%, 90%, 91%, 92%, 93%, 94%, 95%, 96%, 97%, 98%, 99% or higher identity to its references polypeptide

In some embodiments, a variant comprises one or more conservative or otherwise non-disruptive modifications (e.g., substitutions, deletions or additions) relative to its reference. In some embodiments, a variant is free of any disruptive modifications (e.g., substitutions, deletions or additions) so that an immunomodulatory polypeptide maintains one or more functional characteristics of the reference. In some embodiments, maintains means an immunomodulatory polypeptide display comparable activity (e.g., signaling competency or binding) compared to an appropriate reference standard (e.g., a wild-type immunomodulatory polypeptide). For example, in some such embodiments, an immunomodulatory polypeptide maintains at least about 70%, 75%, 80%, 85%, 90%, 91%, 92%, 93%, 94%, 95%, 96%, 97%, 98%, 99% or higher activity compared to an appropriate reference standard (e.g., a wild-type immunomodulatory polypeptide).

### Metal Hydroxides

In some embodiments, the present disclosure provides phosphorylated fusion polypeptides comprising an immunomodulatory polypeptide and a metal-hydroxide binding polypeptides, wherein the phosphorylated fusion polypeptide, when exposed to a metal-hydroxide forms a complex therewith. A complex is formed via adsorption of the phosphorylated fusion polypeptide to a metal-hydroxide. Without wishing to be bound by any one theory, it is hypothesized adsorption of a phosphorylated fusion polypeptide to a metal hydroxide occurs by ligand exchange. Ligand exchange, for example, is a substitution or exchange of a surface hydroxyl by another ligand. In some embodiments, substitution or exchange of a surface hydroxyl occurs by a hydroxyl-replacement group (e.g., a phosphate group).

In some embodiments, a metal-hydroxide is a substance that includes at least one hydroxyl group bound to a metal. In accordance with the present disclosure, in some embodiments, a metal-hydroxide can adsorb fusion polypeptides comprising a hydroxyl-replacement moiety. In some embodiments, a hydroxyl-replacement moiety is a phosphate group.

In some embodiments, a metal-hydroxide is selected based on its inherent qualities or characteristics. In some embodiments, a metal-hydroxide is selected due to its biocompatibility for use in a subject (e.g., a mammal, e.g., a human). In some embodiments, a metal-hydroxide is aluminum hydroxide (e.g., alum). In some embodiments, a metal-hydroxide is iron-hydroxide. A skilled artisan will recognize any number of metal-hydroxide may be successfully utilized in accordance with the present disclosure.

In some embodiments, an aluminum hydroxide is formulated in a gel (e.g., aluminum hydroxide gel). In some embodiments, an aluminum hydroxide is formulated in water. In some embodiments, the concentration of a stock aluminum hydroxide preparation is 10 mg/mL.

### Manufacturing Phosphorylated Fusion Polypeptides

In one aspect of the present disclosure, a phosphorylated form of a fusion polypeptide as described herein is produced by a method comprising a step of (1) producing a phosphorylated form of a fusion polypeptide in a host cell.

In one aspect of the present disclosure, a high purity preparation of a phosphorylated form of a fusion polypeptide from a cell extract that comprises the phosphorylated is obtained by a method comprising a step of (2) purifying said fusion polypeptide from the cell extract (e.g., of step (1)).

In one aspect of the present disclosure, a fusion polypeptide metal-hydroxide complex comprising a phosphorylated form of a fusion polypeptide as described herein is produced by a method comprising a step of (3) contacting a phosphorylated form of a fusion polypeptide (e.g., of step (2)) with a metal hydroxide.

### Expression in Host Cells

In some embodiments of the present disclosure, fusion polypeptides are manufactured by production in a host cell, such as a mammalian cell. Typically, such host cell (e.g., such mammalian cell) will have been engineered to express the fusion polypeptide. Those skilled in the art will be familiar with a variety of technologies for introducing exogenous genetic sequences (e.g., encoding a fusion polypeptide, and/or a kinase) into a host cell, such as a mammalian host cell, for expression thereby.

For example, in some embodiments, a polynucleotide (e.g., DNA or RNA) encoding a fusion polypeptide of the present disclosure may be prepared, e.g., for introduction into a host cell. For example, sequences encoding fusion polypeptides may be excised from DNA using restriction enzymes, may be amplified from plasmids or genomic polynucleotide sequences using, for example, polymerase chain reaction, or may be synthesized using chemical synthesis techniques. In some embodiments, a combination of known methods is utilized to prepare a recombinant polynucleotide encoding fusion polypeptides of the present disclosure.

Recombinant polynucleotides encoding fusion polypeptides of the present disclosure may be cloned into a vector capable of expressing a fusion polypeptide. Cloning may be carried out according to a variety of methods available (e.g., Gibson assembly, restriction digest and ligation, etc.). In some embodiments, a vector is a viral vector. In some embodiments, a vector is a non-viral vector. In some embodiments, a vector is a plasmid. In some embodiments, a vector is a transposon.

In some embodiments, a vector capable of expression comprises a recombinant polynucleotide that encodes a fusion polypeptide of the present disclosure is operatively linked to a sequence or sequences that regulates expression of the polynucleotides (e.g., promoters, start signals, stop signals, polyadenylation signals, activators, repressors, etc.). In some embodiments, a regulatory sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of recombinant polynucleotides that encode a plurality polypeptides. In some embodiments, a plurality of recombinant polypeptides are expressed from the same vector (e.g., a bi-cistronic vector, a tri-cistronic vector, multi-cistronic.). In some embodiments, a plurality of recombinant polypeptides are expressed, each of which is expressed from a separate vector.

In some embodiments, a vector capable of expression comprising a recombinant polynucleotide encoding a fusion polypeptide of the present disclosure is used to express a fusion polypeptide in a host cell.

A host cell may be selected from a variety of the available and known host cells (e.g., Human Embryonic Kidney (HEK) cells, suspension HEK293 cells, Chinese Hamster Ovary cells) suitable expressing fusion polypeptides disclosed herein.

A variety of methods are available to introduce a vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, host cells (e.g., producer cells) are used to manufacture a phosphorylated form of a fusion polypeptide.

In some embodiments, host cells expressing a fusion polypeptide and/or kinase are cultured in a single use bioreactor (e.g., 50 L to 4000L) or a stainless steel bioreactor (e.g., 50 L to 4000L). In some embodiments, host cell are cultured at a temperature ranging from 30° C. to 40° C. In some embodiments, the temperature is lower during the production phase (e.g., 33° C.). In some embodiments, the cell extract is harvested by a 2 or 3 stage filters followed by terminal sterile 0.22 μm filtration.

In some embodiments, a nucleic acid encoding a fusion polypeptide is introduced into a host cell, such that the fusion polypeptide is expressed by the host cell. Alternatively or additionally, in some embodiments, a nucleic acid encoding a kinase that phosphorylates a fusion polypeptide is introduced into a host cell so that the host cell expresses the kinase. In many embodiments, as described herein, both a nucleic acid encoding a fusion polypeptide and a nucleic acid encoding a kinase that phosphorylates it are introduced into the same host cell; in some such embodiments, a single nucleic acid molecule may encode both.

In some embodiments, a nucleic acid molecule introduced into a cell is an RNA (e.g., an mRNA); in some such embodiments, encoded polypeptide(s) (e.g., a fusion polypeptide and/or a kinase) is/are expressed from such RNA. Alternatively or additionally, in some embodiments, a nucleic acid molecule introduced into a cell is a DNA (e.g., a single stranded DNA or a double stranded DNA). In some embodiments, a nucleic acid is introduced into a cell so that coding sequences integrate into the host cell (e.g., into its genome); in some such embodiments encoded polypeptide(s) (e.g., a fusion polypeptide and/or a kinase) is/are expressed therefrom,

In some embodiments, a nucleic acid molecule introduced into a cell (e.g., a nucleic acid molecule that encodes a fusion polypeptide and/or a kinase) includes one or more expression elements, e.g., that may regulate expression of such encoded polypeptide(s). Alternatively or additionally, in some embodiments, a nucleic acid molecule introduced into a cell (e.g., a nucleic acid molecule that encodes a fusion polypeptide and/or a kinase) may be designed or intended to become associated (e.g., by integration) with one or more regulatory elements in a host cell.

In some embodiments, a vector (e.g., a transposon) that includes sequences encoding a fusion polypeptide and/or a kinase as described herein is used to express a fusion polypeptide and/or a kinase in a host cell.

In some embodiments, a host cell may be selected from a variety of the available and known host cells (e.g., Human Embryonic Kidney (HEK) cells, suspension HEK293 cells, Chinese Hamster Ovary cells) suitable expressing fusion polypeptides disclosed herein. In some embodiments, a host cell is a mammalian cell.

A variety of methods are available to introduce a nucleic acid (e.g., a vector, such as an expression vector) into host cells. In some embodiments, a nucleic acid may be introduced into host cells using transfection. In some embodiments, transfection is completed, for example, using calcium phosphate transfection, lipofection, or polyethylenimine-mediated transfection. In some embodiments, a nucleic acid may be introduced into a host cell using transduction. In some embodiments, a nucleic acid may be introduced into a host cell using electroporation. In some embodiments, a nucleic acid may be introduced into a host cell using particle delivery, e.g., polymer particle delivery, lipid particle delivery, gold particle delivery, etc.

### Phosphorylation

In some embodiments, the present disclosure provides a method of manufacturing a phosphorylated form of fusion polypeptides disclosed herein by contacting the fusion polypeptide with a kinase. In some embodiments, a nucleic acid, such as a nucleic acid encoding a fusion polypeptide and/or a kinase is introduced into a host cell. In some embodiments, a fusion polypeptide is contacted with a kinase by co-expressing a fusion polypeptide in a host cell with a kinase. In some embodiments, co-expression is achieved by introducing two vectors, one comprising a recombinant polynucleotide encoding a fusion polypeptide and one comprising a recombinant polynucleotide encoding a kinase, into a host cell. In some embodiments, co-expression is achieved by introducing a single, multi-cistronic (e.g., bi-cistronic) vector that comprises a plurality of recombinant polynucleotides (e.g., such as a transposon). In some embodiments, a recombinant polynucleotide encodes a fusion polypeptide and a recombinant polynucleotide encodes a kinase. In some embodiments, a transformed host cell is cultured following introduction of a vector (e.g., a transposon) into a host cell. Without wishing to be bound by any one theory, upon co-expression of a fusion polypeptide and a kinase in a host cell, a kinase can contact a fusion polypeptide and phosphorylate it.

In some embodiments, co-expression is achieved by introducing two vectors, one comprising a recombinant polynucleotide encoding a fusion polypeptide and one comprising a recombinant polynucleotide encoding a kinase, into a host cell. In some embodiments, two vectors are introduced at a ratio of vector encoding fusion polypeptide to vector encoding a kinase introduced into a host cell optimized to achieve a desired, relative level of expression of fusion polypeptide to kinase. In some embodiments, a ratio of vector encoding fusion polypeptide to vector encoding a kinase is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1.

In some embodiments, co-expression is achieved by introducing one vector comprising both a recombinant polynucleotide encoding a fusion polypeptide and a recombinant polypeptide encoding a kinase (e.g., a bi-cistronic vector) into a host cell. In some embodiments, co-expression is achieved by introducing one transposon comprising both a recombinant polynucleotide encoding a fusion polypeptide and a recombinant polypeptide encoding a kinase into a host cell. In some embodiments, a transposon is a DNA transposon. In some embodiments, the transposon or part thereof (e.g., comprising nucleotides encoding the fusion polypeptide and nucleotides encoding the kinase) is integrated in the host cell genome by an integration enzyme (i.e., by an integrase enzyme, such as a DDE/D integrase enzyme). In some embodiments, an integration enzyme is delivered to the host cell as mRNA. In some embodiments, an integration enzyme is a PiggyBac enzyme. In some embodiments, an integration enzyme is a Leap-In Transposase. In some embodiments, a transposon or part thereof is not integrated into the genome by random integration. In some embodiments, a single copy of a polynucleotide encoding a fusion polypeptide of the present disclosure is integrated into specific multiple host cell genomic loci. In some embodiments, the integration of a polynucleotide encoding a fusion polypeptide of the present disclosure is irreversible. Irreversible integration of the fusion polypeptide into the host cell genome may ensure stable integration. Hereby allowing production of a very stable cell line. In some embodiments, a recombinant polynucleotide encoding a fusion polypeptide and a recombinant polynucleotide encoding a kinase are operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, multiple regulatory nucleotide sequences that control expression (e.g., promoters) are utilized to achieve a desired ratio of expression of fusion polypeptide to kinase. In some embodiments, a ratio is 1:1, 2:1, 3:1,4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1. In some embodiments, a regulatory nucleotide sequence is a promoter. In some embodiments, a specific ratio (e.g., 2:1, 4:1, 8:1 or 15:1) is achieved using a single transposon with two promoters to express the fusion polypeptide and the kinase. In some embodiments, a single separate transposon comprises promoters of differing strength to produce the desired ratio (e.g., 8:1). In some embodiments, the promoter is a CMV or EF1a promoter. In some embodiments, a fusion polypeptide is under the control of a CMV promoter or EF1a promoter. In some embodiments, the promoter is a SV40 or Ubc promoter. In some embodiments, a kinase is under the control of a SV40 promoter or Ubc promoter. In some embodiments, a ratio of fusion polypeptide to kinase is 8:1.

In some embodiments, transformed host cells (i.e., host cells into which a nucleic acid, such as a nucleic acid encoding a fusion polypeptide and/or a kinase) are cultured following such transformation, for example to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. In some embodiments, a transformed host cells are cultured for at least 1 day, 2 days, 3 days, 4 days, 5 days, 6 days, 7 days, 8 days, 9 days, 10 days, 11 days, 12 days, 13 days, 14 days or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art. In some embodiments, host cells secret a phosphorylated form of a fusion polypeptide to the cell extract. In some embodiments, a host cell may show increased secretion of phosphorylated fusion polypeptides to the cell extract compared to an unphosphorylated fusion polypeptides. In some embodiments, a host cell secrets comparable levels of phosphorylated fusion polypeptide and unphosphorylated fusion polypeptides to the cell extract. In some embodiments, a cell extract comprises an unphosphorylated form of a fusion polypeptide. In some embodiments, a cell extract comprises a phosphorylated form of the fusion polypeptide. In some embodiments, a cell extract comprises a mixture of both unphosphorylated and phosphorylated forms of a fusion polypeptide. In some embodiments, a cell extract comprises more phosphorylated forms of a fusion polypeptide than unphosphorylated forms of a fusion polypeptide.

In some embodiments, a host cell secretes a phosphorylated form of a fusion polypeptide to the cell extract, but does not secrete a kinase. In some embodiments, a host cell does not secrete a kinase or only secretes a small amount of a kinase.

In some embodiments, a host cell extract comprises a phosphorylated form of a fusion polypeptide. In some embodiments, a host cell extract comprises host cell proteins and/or host cell nucleotides.

In some embodiments, a phosphorylated form of a fusion polypeptide is harvested from transformed host cells and clarified by centrifugation.

In some embodiments, a transformed host cell is characterized in that a culture thereof produces the fusion protein with a titer of at least 200 mg/L, such as at least 250 mg/L, such as at least 300 mg/L, such as at least 350 mg/L, such as at least 400 mg/L, such as at least 450 mg/L, such as at least 500 mg/L, such as at least 550 mg/L, such as at least 600 mg/L, such as at least 650 mg/L, such as at least 700 mg/L, such as at least 750 mg/L, such as at least 800 mg/L, such as at least 850 mg/L, such as at least 900 mg/L, such as at least 950 mg/L, such as at least 1 g/L or more.

In some embodiments, one or more serine residues at position 43, 281, 306, 311, 316, 365 or 481 of SEQ ID NO: 2 are phosphorylated. In some embodiments, one or more serine residues at position 43, 154, 281, 306, 311, 316, 365 or 481 of SEQ ID NO: 2 are phosphorylated. In some embodiments, one or more serine residues at position 43, 154, 168, 281, 306, 311, 316, 365 or 481 of SEQ ID NO: 2 are phosphorylated. In some embodiments, one or more serine residues at position 43, 154, 168, 281, 306, 311, 316, 365, 406 or 481 of SEQ ID NO: 2 are phosphorylated. In some embodiments, at least serine residue at position 481 of SEQ ID NO: 2 is phosphorylated.

### Purification

In some embodiments, the present disclosure provides technologies (e.g., manufacturing technologies) that are or comprise methods of purification—e.g., methods that comprise one or more purification steps. In some embodiments, a phosphorylated form of a fusion protein is purified from an extract of cell described herein.

In some embodiments, a purification step may include removal of commonly aberrant products (e.g., residual protein, host cell contaminants (e.g., host DNA and/or protein, etc.)) from a cell extract.

In some embodiments, a manufacturing method comprising one or more purification steps results in a high purity preparation of a phosphorylated form of a fusion polypeptide. In some embodiments, a high purity preparation of a phosphorylated form of a fusion polypeptide comprises reduced host cell proteins comparable to a cell extract described herein above. In some embodiments, such high purity preparation does not comprise any host cell proteins. In some embodiments, a high purity preparation comprises less than 100 ng/mg host cell proteins, such as less than 50 ng/mg, such as less than 40 ng/mg, such as less than 30 ng/mg, such as less than 20 ng/mg, such as less than 10 ng/mg, such as less than 9 ng/mg, such as less than 8.5 ng/mg host cell proteins. In some embodiments, a high purity preparation of a phosphorylated form of a fusion polypeptide comprises reduced host cell DNA comparable to a cell extract described herein above. In some embodiments, such high purity preparation does not comprise any host cell DNA. In some embodiments, a high purity preparation comprises less than 10 pg/mg host cell DNA, such as less than 9 pg/mg, such as less than 8 pg/mg, such as less than 7 pg/mg, such as less than 6 pg/mg, such as less than 5 pg/mg, such as less than 4 pg/mg, such as less than 3 pg/mg, such as less than 2 pg/mg, such as less than 1.5 pg/mg, such as less than 1 pg/mg, such as less than 0.9 pg/mg, such as less than 0.8 pg/mg, such as less than 0.7 pg/mg host cell DNA.

In some embodiments, a high purity preparation comprises a low degree of in-process compounds. In some embodiments, in-process compounds may be Tropolone, Pluronic, PDMS, Octamethylcyclo tetrasiloxane D4, TDAO, and/or Fam20). In some embodiments, a high purity preparation comprises less than 1 mg/mL TDAO, such as less than 0.9 mg/mL TDAO such as less than 0.8 mg/mL TDAO such as less than 0.7 mg/mL TDAO, such as less than 0.6 mg/mL TDAO, such as less than 0.5 mg/mL TDAO, such as less than 0.4 mg/mL TDAO, such as less than 0.3 mg/mL TDAO. In some embodiments, a high purity preparation comprises less than 5000 ng Fam20C/mg IL-12 fusion polypeptide, such as less than 4000 ng Fam20C/mg IL-12 fusion polypeptide, such as less than 3000 ng Fam20C/mg IL-12 fusion polypeptide, such as less than 2500 ng Fam20C/mg IL-12 fusion polypeptide, such as less than 2000 ng Fam20C/mg IL-12 fusion polypeptide, such as less than 1800 ng Fam20C/mg IL-12 fusion polypeptide, such as less than 1000 ng Fam20C/mg IL-12 fusion polypeptide, such as less than 750 ng Fam20C/mg IL-12 fusion polypeptide, such as less than 500 ng Fam20C/mg IL-12 fusion polypeptide, such as less than 300 ng Fam20C/mg IL-12 fusion polypeptide, such as less than 200 ng Fam20C/mg IL-12 fusion polypeptide, such as less than 100 ng Fam20C/mg IL-12 fusion polypeptide, such as less than 80 ng Fam20C/mg IL-12 fusion polypeptide, such as less than 70 ng Fam20C/mg IL-12 fusion polypeptide, such as less than 60 ng Fam20C/mg IL-12 fusion polypeptide.

In some embodiments, a phosphorylated form of a fusion polypeptide can be purified by a including one or more chromatography purification steps. In some embodiments, one or more conventional chromatography steps is used. In some embodiments, a conventional chromatography step utilizes anion or cation exchange, hydrophobic interactions, hydroxyapatite chromatography.

In some embodiments, a phosphorylated form of a fusion polypeptide can be purified by including one or more purification steps selected from an ion chromatography step (e.g., an anion chromatography step) and a hydrophobic interaction chromatography step. Accordingly, resulting in separation of the phosphorylated fusion polypeptide from impurities.

Those skilled in the art will be familiar with a variety of purification (e.g., chromatography) substrates, and formats thereof that can be utilized in accordance with the present disclosure. For example, in some embodiments, beads, particles, microspheres, resins, etc. may be utilized. In some embodiments, a substrate utilized for purification (e.g., for chromatography) has properties such that, in accordance with the present disclosure, permits a different retention time for a fusion polypeptide relative to any other undesirable components in the preparation of fusion polypeptide.

In some embodiments, a phosphorylated form of a fusion polypeptide is not purified by an affinity-based purification method. For example, in some embodiments, provided purification technologies do not utilize affinity chromatography.

In some embodiments, a phosphorylated form of a fusion polypeptide may be eluted from a solid substrate. In some embodiments, elution may be carried out using specific elution. For example, in some embodiments specific elution is completed by challenging a polypeptide-substrate complex by an agent or agents that will complete for complexation with either a substrate or a polypeptide, releasing a polypeptide into solution. In some embodiments, elution may be carried out using non-specific elution. For example, in some embodiments, non-specific elution is completed by manipulating solvent or buffer conditions (e.g., increasing concentration of a buffer, e.g., an imidazole buffer) to reduce the associate rate constant, resulting in dissociation of the polypeptide from the substrate.

### First Chromatography Step

In some embodiments, methods according to the present disclosure comprise at least one ion chromatography step (e.g., an anion chromatography step). In some embodiments, methods according to the present disclosure comprise at least one anion chromatography step.

In some embodiment, a first chromatography step is a capturing step (e.g., an anion chromatography capturing step). Without wishing to be bound by any one theory, polypeptide phosphorylation confers variability in a polypeptide's charge, permitting separation of differentially phosphorylated polypeptides using ion-exchange chromatography (e.g., anion-exchange chromatography). Anion-exchange chromatography is a form of ion exchange where a negatively charged biomolecule (e.g., a phosphorylated form of a fusion polypeptide disclosed herein) binds to a positively charged solid substrate (e.g., resin). The positively charged solid substrate hereby captures the negatively charged fusion polypeptide from the cell extract and at the same time remove positively charged impurities from the cell extract as well as fusion polypeptide aggregates, as aggregates seem to have a less negatively charge.

In some embodiments, anion-exchange chromatography can be used to resolve polypeptides with different numbers of phosphorylated amino acid residues (e.g., differentially phosphorylated polypeptides). Anion exchange chromatography can enrich a fusion polypeptide preparation for highly phosphorylated species (e.g., fusion polypeptides having more than 6 phosphorylation sites). Accordingly, in some embodiments, a preparation having a high concentration of phosphorylated fusion polypeptide and a low concentration of positively charged impurities is generated through use of an anion-exchange chromatography step (e.g., as a first step).

In some embodiments, a step of purifying a phosphorylated form of a fusion polypeptide from a cell extract comprises an anion chromatography capture step. In some embodiments, an anion chromatography capture step is a first capture step.

In some embodiments, anion exchange chromatography utilizes an ion exchange resin with covalently bound positively charged groups, such as quaternary amino groups. Commercially available anion exchange resins include Q Sepharose, DEAE Sepharose, TMAE, GigaCap Q 650M and 650S. Binding a negatively charged biomolecule (e.g., a phosphorylated form of a fusion polypeptide) to an anion exchange material comprises, in some embodiments, exposing the negatively charged biomolecule to the resin under appropriate conditions (e.g., pH/conductivity) hereby immobilizing the biomolecule to the anion exchange resin through ionic interactions between the negatively charged biomolecule and a charged group or charged groups of the ion exchange material.

A wash step may entail passing an appropriate buffer through the chromatography resin to wash out unwanted material such as host cell proteins or host cell nucleotides. In some embodiments, a wash buffer may include varying conditions such as pH, conductivity, with the goal of dissociating impurities that are non-specifically bound with the chromatography resin. In some embodiments, a wash step utilizes a mixture of an equilibration and an elution buffer.

Phosphorylated forms of fusion polypeptides can be eluted from a solid substrate (e.g, a positively charged resin) using an elution. In some embodiments, a negatively charged agent (e.g., a phosphorylated form of a fusion polypeptide) is eluted using a buffer that decreases interaction between the anion exchange resin and the negatively charged agent (e.g., a phosphorylated fusion polypeptide). In some embodiments, such an elution buffer may have higher concentration of salt and/or different pH so that dissociation of the negatively charged agent from the chromatography resin is promoted.

In some embodiments, a gradient elution buffer (e.g., a buffer with increasing salt concentrations) is used to elute from the ion exchange (e.g., anion exchange) column. In some such embodiments, use of such a gradient may permitting separation of differently phosphorylated polypeptides (i.e., may achieve separation of different phosphoforms).

In some embodiments, a buffer is, for example, a Tris buffer. In some embodiments, a linear gradient of Tris buffer is utilized. In some embodiments, a linear gradient of Tris buffer comprises over a linear gradient from 20 mM Tris, pH 7.1 to 20 mM Tris, 1 M NaCl, pH 7.1 over a pre-defined period of time. In some embodiments, a linear gradient is conducted over a period of 1 minute, 5 minutes, 10 minutes, 15 minutes, 20 minutes, 22 minutes, 24 minutes, 26 minutes, 28 minutes, 30 minutes, 32 minutes, 34 minutes, 36 minutes, 38 minutes, 40 minutes, or longer. In some embodiments, a first anion chromatography capture step uses a Tris buffer. In some embodiments, a first anion chromatography capture step is performed at a pH of about 6 to about 9, such as about 7 to about 8.

In some embodiments, a first anion chromatography capture step is performed using capturing beads. In some embodiments, a first step capture bead has a diameter of at least 50 μm, such as at least 55 μm, such as at least 60 μm, such as 65 at least μm, such as at least 70 μm, such as at least 75 μm. In some embodiments, a first capture bead is a GigaCap Q 650M.

In some embodiments, a first anion chromatography capture step comprises an immobilizing step (e.g., binding a phosphorylated fusion polypeptide to a chromatography column), a pre-elution wash step, and an elution step (e.g., eluting a phosphorylated fusion polypeptide). In some embodiments, a resin bead of about 50 to about 100 micrometer particle size (mean), such as 75 micrometer particle size (mean) is utilized for immobilizing a phosphorylated fusion polypeptide. In some embodiments, an immobilization composition having a low salt concentration is used during immobilization of a phosphorylated fusion polypeptides to a chromatography column (e.g., 0 M sodium chloride is used). In some embodiments, a pre-elution composition having an intermediate salt concentration, comparable to the salt concentration of the immobilization composition and the elution composition (e.g., 215 mM sodium chloride is used) is utilized during a pre-elution wash step. In some embodiments, an elution composition having a high salt concentration is used during the elution of the phosphorylated fusion polypeptide from the chromatography column (e.g., 350 mM sodium chloride is used). In some embodiments, a first anion chromatography capture step is performed at a pH within the range of about 7 to about 8 (e.g., 7.4). In some embodiments, a flow rate of 200-400 cm/h is used (e.g., 300 cm/h).

### Second Chromatography Step

In some embodiments, methods according to the present disclosure comprise at least one hydrophobic interaction chromatography step. In some embodiments, a hydrophobic interaction step is performed after a first anion chromatography step. Without being bound to a particular theory, a phosphorylated form of a fusion protein according to the present disclosure has low hydrophobicity (e.g., due to its phosphorylation degree and hence its charge) which can be used to separate it from hydrophobic impurities. In some embodiments, hydrophobic host cell impurities (e.g., host cell proteins) and/or fusion polypeptide aggregates are separated from a preparation of a phosphorylated form of a fusion polypeptide. In some embodiments, a preparation (e.g., a high purity preparation) comprises less than 5% aggregated fusion polypeptides, such as less than 4%, such as less than 3%, such as less than 3%, such as less than 2%, such as less than 1%, such as less than 0.9%, such as less than 0.8% aggregated fusion polypeptides.

A hydrophobic interaction step may, in some embodiments, separate product or process related impurities, e.g., hos cell proteins or aggregated product (e.g., fusion polypeptide aggregates) from a phosphorylated form of a fusion polypeptide based on differences in hydrophobic interactions of the phosphorylated fusion polypeptide and the impurities with a hydrophobic material. Such a step may in some embodiments be referred to as a polishing step.

Examples of hydrophobic interaction resins include, but are not limited to, hydrophobic ligands such as alkyl groups ranging from 2 to 8 carbon atoms, or aryl groups such as phenyl. Binding a negatively charged agent (e.g., a phosphorylated form of a fusion polypeptide) to a hydrophobic interactions resin comprises, in some embodiments, exposing the biomolecule to the resin under appropriate conditions (pH/conductivity) hereby immobilizing the biomolecule to the hydrophobic resin through hydrophobic interactions between the biomolecule and a non-polar group of the hydrophobic interactions material. Hydrophobic interaction bindings typically happen at a high concentration salt (e.g. 1 to 1.8 M Ammonium Sulfate). In some embodiment, a phosphorylated form of a fusion polypeptide is immobilized at a high salt concentration (e.g., 1.4 M sodium sulfate). In some embodiments, a phosphorylated form of a fusion polypeptide is eluted with a linear gradient ranging from 1.4 M ammonium sulfate to 0 M ammonium sulfate.

A wash step may entail passing an appropriate buffer through the chromatography resin to wash out unwanted material such as less hydrophobic host cell proteins. In some embodiments, a wash buffer may have different pH, so that dissociating of the low hydrophobicity agent from impurities that are non-specifically bound with the chromatography resin is promoted. In some embodiments, a wash step utilizes a mixture of an equilibration and an elution buffer.

Phosphorylated forms of fusion polypeptides can be eluted from a solid substrate (e.g., hydrophobic resin) using an elution. In some embodiments, a low hydrophobicity agent (e.g., a phosphorylated form of a fusion polypeptide) is eluted from a hydrophobic resin using a buffer that decreases interaction between the hydrophobic interactions resin and the negatively charged agent (e.g., a phosphorylated fusion polypeptide). In some embodiments, such an elution buffer may have lower concentrations of salts or changes in pH that promote dissociation of the bio molecule from the chromatography resin. In some embodiment, a phosphorylated form of a fusion polypeptide is eluted at a low salt concentration (e.g., 750 mM sodium sulfate).

In some embodiments, hydrophobic interaction chromatography step comprises an immobilizing step (e.g., binding a phosphorylated fusion polypeptide to a chromatography column), and an elution step (e.g., eluting a phosphorylated fusion polypeptide). In some embodiments, a resin bead of about 50 to about 100 micrometer particle size (mean), such as 75 micrometer particle size (mean) is utilized for immobilizing a phosphorylated fusion polypeptide. In some embodiments, an immobilization composition having a high salt concentration is used during immobilization of a phosphorylated fusion polypeptides to a chromatography column (e.g., 1.4 M ammonium sulfate is used). In some embodiments, an elution composition having a low salt concentration is used during the elution of the phosphorylated fusion polypeptide from the chromatography column (e.g., 740 mM ammonium sulfate is used). In some embodiments, a hydrophobic interaction chromatography step is performed at a pH within the range of about 7 to about 8 (e.g., 7.4). In some embodiments, a flow rate of 200-350 cm/h is used (e.g., 275 cm/h).

### Third Chromatography Step

In some embodiments, methods according to the present disclosure comprise a first anion chromatography step and a second anion chromatography step. In some embodiments, methods according to the present disclosure comprise a first anion chromatography step and a second anion chromatography step, wherein the first anion chromatography step is a capture step and the second anion chromatography step is a polishing step. In some embodiments, a second anion chromatography step is performed after a hydrophobic interaction chromatography step.

In some embodiments, methods according to the present disclosure comprise the following steps:
i) a first anion chromatography step;
ii) a hydrophobic interaction chromatography step; and
iii) a second anion chromatography step.

In some embodiments, a second anion chromatography step is performed using capturing beads. In some embodiments, a second step capture bead has a diameter of at the most 50 μm, the most 45 μm, the most 40 μm, the most 35 μm. In some embodiments, a first capture bead is a GigaCap Q 6505.

In some embodiments, a second anion chromatography capture step comprises an immobilizing step (e.g., binding a phosphorylated fusion polypeptide to a chromatography column), a pre-elution wash step, and an elution step (e.g., eluting a phosphorylated fusion polypeptide). In some embodiments, a resin bead of about 10 to about 50 micrometer particle size (mean), such as 35 micrometer particle size (mean) is utilized for immobilizing a phosphorylated fusion polypeptide. In some embodiments, an immobilization composition having a low salt concentration is used during immobilization of a phosphorylated fusion polypeptides to a chromatography column (e.g., 0 M sodium chloride is used). In some embodiments, a pre-elution composition having an intermediate salt concentration, comparable to the salt concentration of the immobilization composition and the elution composition (e.g., 274 mM sodium chloride is used) is utilized during a pre-elution wash step. In some embodiments, an elution composition having a high salt concentration is used during the elution of the phosphorylated fusion polypeptide from the chromatography column (e.g., 355 mM sodium chloride is used). In some embodiments, a first anion chromatography capture step is performed at a pH within the range of about 7 to about 8 (e.g., 7.3). In some embodiments, a flow rate of 200-400 cm/h is used (e.g., 300 cm/h).

### Optional Additional Step(s)

In some embodiments, methods according to the present disclosure comprise a viral inactivation step or viral removal step. In some embodiments, a viral inactivation step or viral removal step is performed before or after any of the chromatography steps described herein above. In one embodiment, a viral inactivation step or viral removal step is performed prior to the first chromatography step (e.g., a first anion chromatography step). In some embodiments, a viral inactivation step comprises pH inactivation or chemical inactivation (e.g., through use of a chemical agent such as a surfactant). In some embodiments, a viral inactivation step comprises utilizes a detergent as, without being bound a particular theory, IL12-ABP is sensitive and might aggregate at low pH. In some embodiments, a viral inactivation step comprises utilizes a detergent selected from Myristyldimethylamine N-oxide, TDAO, Triton X-100 or Polysorbates. In some embodiments, a viral removal step comprises a filtration step.

### Fusion Polypeptide Preparations

In some embodiments, the present disclosure, among other things, provides fusion polypeptide preparations. In some embodiments, fusion polypeptide preparations comprise a phosphorylated form of a fusion polypeptide. In some embodiments, a fusion polypeptide preparation is a high purity preparation of a phosphorylated form of a fusion polypeptide. In some embodiments, a fusion polypeptide preparation comprises a mixture of both unphosphorylated and phosphorylated forms of a fusion polypeptide. In some embodiments, a fusion polypeptide preparation comprises more fusion polypeptide that are phosphorylated than fusion polypeptide that are unphosphorylated.

In some embodiments, a phosphorylated fusion polypeptide preparation comprises fusion polypeptides with varying degrees of phosphorylation as described herein above.

In some embodiments, fusion polypeptide preparations comprise a buffer. In some embodiments, fusion polypeptide preparations comprise a Tris buffer of about pH 7 to about pH 8. In some embodiments, a fusion polypeptide preparation comprises a salt (e.g., NaCl).

### Fusion Polypeptide Compositions

In some embodiments, the present disclosure, among other things, provides fusion polypeptide compositions. In some embodiments, fusion polypeptide compositions comprise a phosphorylated form of a fusion polypeptide. In some embodiments, a fusion polypeptide composition is a high purity composition of a phosphorylated form of a fusion polypeptide. In some embodiments, a fusion polypeptide composition comprises a mixture of both unphosphorylated and phosphorylated forms of a fusion polypeptide. In some embodiments, a fusion polypeptide composition comprises more fusion polypeptide that are phosphorylated than fusion polypeptide that are unphosphorylated. In some embodiments, a fusion polypeptide composition comprises fusion polypeptides with varying degrees of phosphorylation as described herein above.

In some embodiments, a fusion polypeptide composition comprises a buffer. In some embodiments, a buffer is a Tris buffer. Among other things, the present disclosure demonstrates that Tris buffer(s) are particularly useful for stability of IL-12 fusion polypeptides as described herein. For example, the present disclosure demonstrates that Tris buffer can provide surprising stability advantages relative to alternative buffer(s) such as histidine buffer(s). In some embodiments, a buffer is not a Histidine buffer.

In some embodiments, a fusion polypeptide composition has a pH of about 6.5 to about 8. In some embodiments, a fusion polypeptide composition has a pH of at the most 7.5, such as at the most 7.4, such as at the most 7.7. In some embodiments, a fusion polypeptide composition has a pH of at least 7, such as at least 7.1, such as at least 7.2, such as at least 7.3. Without wishing to be bound by any particular theory, a high pH may lead to deamidation of the IL-12 fusion polypeptide.

In some embodiments, a fusion polypeptide composition comprises a salt. Without wishing to be bound by any particular theory, salt may act as a tonicity modifier in compositions and/or formulations and may assist in the stability of the IL-12 fusion polypeptide by stabilizing the structure of the molecule via ionic interactions. In some embodiments, a fusion polypeptide composition comprises a salt, wherein the concentration of the salt is within the range of about 1 mM and about 750 mM, such as within the range of about 10 mM and about 500 mM, such as within the range of about 20 mM and about 100 mM, such as within the range of about 30 mM and about 60 mM, such as within the range of about 35 mM and about 55 mM. In some embodiments, a salt is NaCl or Na2SO4.

In some embodiments, a fusion polypeptide composition comprises a surfactant. In some embodiments, a fusion polypeptide composition comprises a hydrophilic surfactant. Hydrophilic surfactants may interact more with charged molecules such as a phosphorylated form of an IL-12 fusion polypeptide compared to more hydrophobic surfactants. This interaction might mitigate formation of visible particles upon shaking, i.e. more vulnerable to agitation. In some embodiments, a fusion polypeptide composition comprises a Polysorbate (e.g., Polysorbate 20 or Polysorbate 80). In some embodiments, a fusion polypeptide composition comprises a Polysorbate 20. In some embodiments, a fusion polypeptide composition comprises a surfactant (e.g., Polysorbate 20), wherein the concentration of the surfactant is within the range of about 0.0005% w/v and about 1% w/v, within the range of about 0.005% w/v and about 0.1% w/v, within the range of about 0.01% w/v and about 0.05% w/v, within the range of about 0.015% w/v and about 0.2% w/v.

In some embodiments, a fusion polypeptide composition comprises L-methionine. The addition of methionine seems to have a positive impact on the stability of IL-12 fusion polypeptide, notably mitigating oxidation risks and on preventing development of high molecular weight species (HMWS) and as well as. In some embodiments, a fusion polypeptide composition comprises L-methionine, wherein the concentration of L-methionine is within the range of about 1 mM and about 20 mM, such as within the range of about 5 mM and about 15 mM.

In some embodiments, a fusion polypeptide composition comprises a disaccharide (e.g., sucrose or trehalose). In some embodiments, a fusion polypeptide composition comprises sucrose, wherein the concentration of sucrose is within the range of about 100 mM and about 200 mM.

### Pharmaceutical Compositions

In some embodiments, pharmaceutical formulations comprise a fusion polypeptide-metal hydroxide complex as described herein. Exemplary pharmaceutical compositions are shown in Example 8. Such pharmaceutical formulations can be prepared by mixing a fusion polypeptide composition as described herein above with a metal hydroxide. In some embodiments, a fusion polypeptide metal hydroxide complex as described herein is produced by contacting a fusion polypeptide composition with a metal hydroxide (e.g., an aluminum hydroxide). In some embodiments, an aluminum hydroxide is formulated in a gel. In some embodiments, an aluminum hydroxide is formulated in water. In some embodiments, the concentration of a stock aluminum hydroxide preparation is 10 mg/mL.

In some embodiments, the ratio between the fusion polypeptide and the metal hydroxide is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, and 20:1.

In some embodiments, the fusion polypeptide is contacted with a metal hydroxide for at least 10 minutes, such as at least 15 minutes, such as at least 20 minutes, such as at least 25 minutes, such as at least 30 minutes, such as at least 40 minutes.

In some embodiments, the fusion polypeptide is contacted with a metal hydroxide at a temperature within the range of about 15° C. to about 30° C., such as about 20° C. to about 25° C.

In some embodiments, a pharmaceutical composition comprises the same components as a fusion polypeptide composition and a metal hydroxide. In some embodiments, the concentrations of the components in a pharmaceutical formulation comprises are similar to the concentrations of the components in a fusion polypeptide composition. In some embodiments, the concentrations of the components in a pharmaceutical formulation comprises are lower compared to the concentrations of the components in a fusion polypeptide composition.

In some embodiments, a pharmaceutical formulation comprises 0.25 mg/mL fusion polypeptide, 15 mM Tris buffer, 38 mM NaCl, 7.5 mM L-Methonine, 0.015% polysorbate 20, and 113 mM sucrose, 2.5 mg/mL aluminum hydroxide and wherein the pH of the composition is within the range of 6 and 8.

### Characterization

Among other things, in some embodiments, the present disclosure provides technologies for characterizing fusion polypeptides (e.g., phosphorylated or unphosphorylated preparations thereof) and/or of complexes comprising such fusion polypeptides and metal hydroxides. Characterization may be performed during and/or following production process. In some embodiments, a particular preparation process may be modified or terminated in light of a characterization (e.g., if a particular preparation fails to meet one or more specifications). In some embodiments, such characterization may involve assessment of one or more of metal-hydroxide retention, degree of phosphorylation, heterogeneity of phosphorylation, signaling activity, and/or efficacy.

### Exemplary Characterization of Phosphate Content

In some embodiments, degree of phosphorylation (e.g., of fusion polypeptides of the present disclosure) is characterized. A variety of methods are available for measurement of degree of phosphorylation (e.g., the average number of phosphate molecules per polypeptide). For example, in some embodiments, degree of phosphorylation can be determined by a colorimetric method. In some embodiments, a colorimetric method is or comprises a malachite green assay. Without wishing to be bound by any one theory, a malachite green assay is based on quantification of a green complex formed between Malachite green, molybdate, and free orthophosphate which can be measured (e.g., using a spectrophotometer or plate reader).

In some embodiments, degree of phosphorylation (e.g., the average number of phosphate molecules per polypeptide) is about 4-12, about 5-11, about 6-10, about 7-9, or about 7.5-8.5. In some embodiments, degree of phosphorylation (e.g., the average number of phosphate molecules per polypeptide) is 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 7.10, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, or 11. In some embodiments, degree of phosphorylation (e.g., the average number of phosphate molecules per polypeptide) is 7.0, 7.1, 7.2, 7.3, 7.4, 7.5, 7.6, 7.7, 7.8, 7.9, 7.10, 8.0, or 8.1.

In some embodiments, heterogeneity of phosphorylation of fusion polypeptides of the present disclosure and/or preparations thereof are characterized. In some embodiments, heterogeneity of phosphorylation is a measurement of the degree of phosphorylation within a given preparation of fusion polypeptide. In some embodiments, heterogeneity of phosphorylation is a measurement of the degree of phosphorylation across a plurality of preparations of fusion polypeptide. In some embodiments, heterogeneity of phosphorylation is a measurement of location of particular phosphate groups on a polypeptide within a given preparation of fusion polypeptide. In some embodiments, heterogeneity of phosphorylation is a measurement of location of particular phosphate groups on a polypeptide across a plurality of preparations of fusion polypeptide.

A variety of technologies is available for measurement of heterogeneity of phosphorylation. For example, in some embodiments, degree of phosphorylation can be determined by a chromatography method. In some embodiments, a chromatography method comprises ion-exchange chromatography. In some embodiments, for example, a chromatography method comprises analytical anion-exchange chromatography. Anion-exchange chromatography is a form of ion exchange where a negatively charged biomolecule (e.g., a phosphorylated form of a fusion polypeptide disclosed herein) binds to a positively charged resin. In some embodiments, anion-exchange chromatography can be used to resolve polypeptides with different numbers of phosphorylated amino acid residues (e.g., differentially phosphorylated polypeptides). Without wishing to be bound by any one theory, polypeptide phosphorylation confers variability in a polypeptide's charge, permitting separation of differentially phosphorylated polypeptides using ion-exchange chromatography (e.g., anion-exchange chromatography). Use of a gradient elution buffer (e.g., a buffer with increasing salt concentrations) to elute from the ion exchange (e.g., anion exchange) column permits separation of differentially phosphorylated polypeptides. In some embodiments, a buffer is, for example, a Tris buffer. In some embodiments, a linear gradient of Tris buffer is utilized. In some embodiments, a linear gradient of Tris buffer comprises over a linear gradient from 20 mM Tris, pH 7.1 to 20 mM Tris, 525 mM NaCl, pH 7.1 over a pre-defined period of time. In some embodiments, a linear gradient is conducted over a period of 1 minute, 5 minutes, 10 minutes, 15 minutes, 20 minutes, 22 minutes, 24 minutes, 26 minutes, 28 minutes, 30 minutes, 32 minutes, 34 minutes, 36 minutes, 38 minutes, 40 minutes, or longer.

In some embodiments, differentially phosphorylated polypeptides are dephosphorylated. In some embodiments, dephosphorylation comprises use of a phosphatase (e.g., a lambda phosphatase). In some embodiments, a fusion polypeptide is incubated with a phosphatase for a period of time and at a temperature that permits activity of said phosphatase and dephosphorylation of said fusion polypeptide. In some embodiments, dephosphorylation occurs at an incubation temperature of approximately 25° C., 26° C., 27° C., 28° C., 29° C., 30° C., 31° C., 32° C., 33° C., 34° C., 35° C., or higher. In some embodiments, dephosphorylation occurs for an incubation time of 25 minutes, 30 minutes, 35 minutes, 40 minutes, 45 minutes, 50 minutes, 55 minutes, 60 minutes, 65 minutes, or longer. In some embodiments, dephosphorylation occurs for an incubation time of 25-65 minutes, 30-60 minutes, 35-55 minutes, 40-50 minutes, 30-65 minutes, 35-65 minutes, 40-65 minutes, 45-65 minutes, 50-65 minutes, or 55-65 minutes.

In some embodiments, differentially phosphorylated polypeptides are dephosphorylated prior to separation. In some embodiments, differentially phosphorylated polypeptides of the present disclosure are assessed relative to an appropriate reference standard (e.g., a dephosphorylated and/or non-phosphorylated form of a fusion polypeptide).

In some embodiments, after separation of differentially phosphorylated polypeptides (e.g., by ion-exchange chromatography), the amount of each differentially phosphorylated polypeptide is measured. In some embodiments, the amount of each differentially phosphorylated polypeptide is measured according to a variety of methods available in the art. In some embodiments, for example and without limitation, differentially phosphorylated polypeptides are measured using a malachite green assay, analytical ion exchange, spectrophotometer, colorimetric assays, and/or western blot.

### Exemplary Characterization of Metal-Hydroxide Retention

In some embodiments, fusion polypeptides of the present disclosure, when exposed to a metal-hydroxide (e.g., aluminum hydroxide) forms a complex therewith. In some embodiments, retention of a fusion polypeptide of the present disclosure on a metal-hydroxide (e.g., metal-hydroxide retention) is characterized. A variety of methods are available to measure metal-hydroxide retention. In some embodiments, for example and without limitation, metal-hydroxide retention can be measured by ellipsometry, surface plasmon resonance, optical waveguide lightmode spectroscopy, attenuated total internal reflectance-infrared spectroscopy, circular dichroism spectroscopy (CD), total internal reflectance-infrared spectroscopy (TIRF), and other high resolution microscopy techniques.

In some embodiments, metal-hydroxide retention is characterized using an in vitro assay. For example, fusion polypeptides at a known concentration are mixed with an excess of metal-hydroxide. The concentration of free, non-complexed fusion polypeptides is quantified and compared to a standard curve to determine metal-hydroxide retention. The concentration of free, non-complexed fusion polypeptide can be assessed according to a variety of method known to those of skill in the art. For example, and without limitation, in some embodiments, free, non-complexed, fusion polypeptides are quantified by enzyme-linked immunosorbent assay (ELISA), western blot, bicinchoninic acid assay, or Bradford assay.

In some embodiments, at least 10%, 15%, 20%, 25%, 30%, 35%, 40%, 45%, 50%, 55%, 60%, 65%, 70%, 75%, 80%, 85%, 90%, 95%, 99%, or 100% of fusion-polypeptide, when mixed with a metal-hydroxide, forms a complex therewith (e.g., is retained).

### Exemplary Characterization of Signaling Activity

In some embodiments, fusion polypeptides (and/or complexes thereof) as described herein are characterized for activity (e.g., signaling activity). In some embodiments, activity is characterized by assessing signaling activity (e.g., signaling competency) compared to an appropriate reference standard. An appropriate reference standard can be, for example, a wild-type polypeptide and/or a fusion polypeptide lacking a metal-hydroxide binding polypeptide.

A variety of methods are available to assess signaling competency. In some embodiments, for example, signaling competency is assessed using an in vitro- or in vivo-based activity assay.

In some embodiments, signaling activity is assessed with an in vitro activity assay. In some embodiments, an in vitro activity assay comprises measuring activation or inhibition of downstream signaling of a fusion polypeptide. In some embodiments, measuring activation or inhibition of downstream activity comprises use of a reporter (e.g., a reporter assay). In some embodiments, a reporter assay measures activity using a detectable molecule (e.g., a reporter) that correlates with fusion polypeptide activity.

In some embodiments, a reporter comprises a fluorescent, bioluminescent, and/or other detectable probe known to those of skill in the art. In some embodiments, a reporter comprises use of a gene reporter. A gene reporter, for example, can be activated upon signaling elicited from a polypeptide. For example, upon activation of gene reporter transcription, a detectable product or enzyme that can be activated upon addition of substrate, generating a detectable product and/or by-product, can be utilized. In some embodiments, an enzyme useful in accordance with a reporter assay is, for example, luciferase or an alkaline phosphatase (e.g., secreted alkaline phosphatase, SEAP). In some such embodiments, a HEK-blue-IL12 reporter assay is utilized.

In some embodiments, signaling activity is assessed with an in vivo activity assay. In some embodiments, a fusion polypeptide is administered to a subject (e.g., a mouse, non-human primate, human, etc.) and activity is assessed. In some embodiments, activity is assessed, for example, by measuring activation or inhibition of downstream signaling of a fusion polypeptide as compared to an appropriate reference standard (e.g., activity of a wild-type polypeptide). A variety of methods are available to measure activation or inhibition of downstream signaling of a fusion polypeptide. For example, and without limitation, differential gene expression, protein expression, and/or alterations in post-translational modifications induced by a fusion polypeptide can be measured.

### Exemplary Efficacy Characterization

In some embodiments, efficacy can be characterized according to a variety of methods that are available. In some embodiments, for example, a fusion polypeptide (or complex thereof) as described herein is administered (e.g., by intratumoral or peritumoral injection) to a subject (e.g., mouse, non-human primate, human, etc.) and efficacy is determined in comparison to an appropriate reference standard. An appropriate reference standard can be, for example, a wild-type polypeptide and/or a polypeptide lacking a metal-hydroxide binding polypeptide, or having a metal-hydroxide binding polypeptide in a non-binding (e.g., non-phosphorylated) state.

In some embodiments, efficacy is determined pre-clinically in an animal model (e.g., in mice, rats, non-human primates, etc.). In some embodiments, a fusion polypeptide is administered (e.g., by intratumoral or peritumoral injection) to an animal model. For example, in some embodiments, an animal model is an animal model with a tumor (e.g., an animal model of cancer). In some embodiments, a cancer animal model is generated by inoculating said animal model with tumor cells. In some embodiments, an animal model is inoculated with tumor cells at the flank region. In some embodiments, an animal model is inoculated with tumor cells in a clinically relevant region (e.g., a mammary fat pad).

In some embodiments, an animal model of cancer is administered a fusion polypeptide of the present disclosure. In some embodiments, an animal model of cancer is administered a reference standard (e.g., a wild-type polypeptide and/or a polypeptide lacking a metal-hydroxide binding polypeptide). In some embodiment, a variety of available, pre-determined measurements for efficacy known in the art, such as, for example, tumor volume and/or percent survival are assessed over time relative to an appropriate reference standard (e.g., a wild-type polypeptide and/or a polypeptide lacking a metal-hydroxide binding polypeptide).

In some embodiments, efficacy of a fusion polypeptide is determined clinically. In some embodiments, a fusion polypeptide is administered (e.g., by intratumoral, peritumoral injection, or into a tumor-draining lymph node) to a subject with a tumor. In some embodiments, a variety of available, pre-determined measurements for efficacy known in the art, such as, for example, tumor volume and/or percent survival are assessed over time relative to a subject with a tumor administered reference standard (e.g., a treatment in the art of known efficacy and/or placebo).

### Use

### Methods of Treatment

In one aspect, the present disclosure relates to methods of treating a subject with a medical condition. In some embodiments, the present disclosure relates to methods of treating a subject with cancer (e.g., a subject with a tumor). In general, methods of treatment are aimed at reducing tumor volume, reducing and/or preventing metastases, prolonging survival, and/or curing the condition. Appropriate subjects or individuals receiving a fusion polypeptide or fusion polypeptide metal hydroxide complex of the present disclosure include, for example, humans or other mammals (e.g., mice, rats, rabbits, dogs, horses, cats, pigs, or non-human primates) that have a tumor (e.g., cancer).

In some embodiments, a method of treating a subject with a tumor comprises a step of: treating a subject with a complex comprising: a fusion polypeptide comprising an immunomodulatory polypeptide that comprises an immune agonist moiety and a metal-hydroxide binding polypeptide and a metal hydroxide. In some embodiments, a method of treating a subject with a tumor comprises administering a fusion polypeptide comprising: an immunomodulatory polypeptide that comprises an immune agonist moiety and a metal-hydroxide binding polypeptide, wherein a fusion polypeptide is formulated with a metal hydroxide.

In some embodiments, a complex as described herein is administered as a monotherapy. In some embodiments, a complex as described herein is administered in combination with a second therapeutic. In some embodiments, a complex as described herein is administered to a subject wherein a subject has received or is receiving therapy with at least one additional therapeutic.

Fusion polypeptides and/or complexes thereof and/or compositions and/or formulations of the present disclosure, among other things, are useful for treating a subject with a tumor. Non-limiting examples of diseases associated with a tumor include cancer (e.g., carcinoma, sarcoma, metastatic diseases or hematopoietic neoplastic disorders). A tumor, including a metastatic tumor, can arise from a plurality of primary tumor types. For example, and without limitation, in some embodiments, a tumor or metastatic tumor can arise from a primary tumor of the kidney (e.g., renal cell carcinoma), head and neck (e.g., head and neck squamous cell carcinoma), prostate, breast (e.g., triple-negative), colon, skin (e.g., melanoma, merkel cell carcinoma, cutaneous T-cell lymphoma, cutaneous squamous cell carcinoma, basal cell carcinoma), lung (e.g., non-small cell lung cancer), and pancreas. Accordingly, fusion polypeptides and preparations thereof disclosed herein, including fusion polypeptide metal-hydroxide complexes and preparations thereof, can be administered to subject who has cancer.

It will be appreciated by those skilled in the art that amounts of a fusion polypeptide-metal hydroxide complex, fusion polypeptide or a preparation thereof sufficient to reduce tumor growth and size, or a therapeutically effective amount, will vary not only on the particular compounds or preparations selected, but also with the route of administration, the nature of the condition being treated, and the age and condition of the patient, and will ultimately be at the discretion of the patient's physician or pharmacist and/or based upon clinical guidelines. The length of time during which the compounds used in the instant method will be given varies on an individual basis and/or be based upon clinical guidelines.

In some embodiments, a method of treating a subject with a tumor (e.g., cancer) comprises a step of treating the subject with a complex comprising a fusion polypeptide comprising an immunomodulatory polypeptide that comprises an immune agonist moiety and a metal-hydroxide binding polypeptide and a metal hydroxide. In some embodiments, a fusion polypeptide and a metal-hydroxide are formulated together. Formulated together, for example, comprises a pre-formed complex of fusion polypeptide and metal-hydroxide. In some embodiments, a fusion polypeptide and metal-hydroxide are mixed immediately prior to administration.

In some embodiments, a method of treating a subject with a tumor (e.g., cancer) comprises treating a subject with a complex wherein a complex is administered by intratumoral injection. In some embodiments, a method of treating a subject with a tumor (e.g., cancer) comprises treating a subject with a complex wherein a complex is administered by peritumoral injection. In some embodiments, a method of treating a subject with a tumor (e.g., cancer) comprises treating a subject with a complex wherein a complex is administered to a tumor-draining lymph node or lymph nodes.

Methods of the present invention often involve administration of a therapeutically effective amount of a particular agent. A therapeutically effective amount is an amount sufficient to achieve (in principle, for a subject of comparable characteristics, such as species, body type, size, extent of disease or disorder, degree or type of symptoms, history of responsiveness, and/or overall health) an intended biological or medical response or therapeutic benefit in a tissue, system or subject. For example, a desirable response may include one or more of: delaying or preventing the onset of a medical condition, disease or disorder, slowing down or stopping the progression, aggravation, or deterioration of the symptoms of the condition, bringing about ameliorations of the symptoms of the condition, and curing the condition.

When combinations of therapeutic agents are administered, the amount of any individual agent required in the combination may be different from the amount required of that same agent to achieve its therapeutic effect alone. In some cases, synergies between or among therapeutic agents used in a combination may reduce amounts required; in other cases, inhibitory interactions may increase amounts required. Thus, in general, therapeutically effective amounts of a combination of agents may utilize different absolute amounts of the agents than constitute therapeutically effective amounts of the agents individually.

### Combination Therapies

In some embodiments, fusion polypeptide metal-hydroxide complexes or formulations thereof as disclosed herein are administered in combination with other therapies. For example, in some embodiments IL-12 complexes are used in combination with another immunotherapy. Exemplary immunotherapies include, but are not limited to, chimeric antigen receptor (CAR) T cell therapy, tumor-associated antigen targeting antibodies, immune checkpoint inhibitors, and cancer vaccines. In some embodiments, by antagonizing such immune checkpoint inhibitors, an immune response is induced or stimulated. The following table provides a list of immune checkpoint inhibitors are suitable for use in the in combination with the pharmaceutical composition of the present disclosure.

A second therapeutic agent may be selected from a variety of available anti-tumor agents known in the art. In some embodiments, a second therapeutic agent is administered prior to administration of a fusion polypeptide metal-hydroxide complex. In some embodiments, a second therapeutic agent is administered concurrently with a fusion polypeptide metal-hydroxide complex. In some embodiments, a second therapeutic agent is administered after administration with a fusion polypeptide metal-hydroxide complex.

For example, in some embodiments, a second therapeutic is radiation (e.g., ionizing radiation). In some embodiments, an amount of ionizing radiation administered is between about 1 Gy and about 1,000 Gy, about 5 Gy and about 900 Gy, about 10 Gy to about 800 Gy, about 10 Gy to about 700 Gy, about 10 Gy to about 600 Gy, about 10 Gy to about 500 Gy, about 10 Gy to about 400 Gy, about 10 Gy to about 300 Gy, about 10 Gy to about 200 Gy, about 10 Gy to about 100 Gy, about 5 Gy and about 15 Gy, between about 7.5 Gy and about 12 Gy, or between about 10 Gy and about 12 Gy. In some embodiments, an amount of ionizing radiation administered is about 12 Gy. In some embodiments, an amount of ionizing radiation is greater than about 10, 20, 30, 40, 50, 60, 70, 80, 90, 100, 200, 300, 400, 500, or 1,000 Gy. In some embodiments, an amount of ionizing radiation is less than about 1,000, 900, 800, 700, 600, 500,400, 300, 200, 100, 90, 80, 70, 60, or 50 Gy.

For example, in some embodiments, a second therapeutic agent is a chemotherapeutic agent. In some embodiments, a chemotherapeutic agent may be a targeted therapy (e.g., BRAF inhibitor, MEK inhibitor, etc.). In some embodiments, a chemotherapeutic agent may be any approved chemotherapeutic agent. For example, and without limitation, a chemotherapeutic agent can be one or more of adriamycin, anastrozole, cyclophosphamide, docetaxel, doxifluridine, doxorubicin, erlotinib, fluorouracil, gemcitabine, imatinib, iressa, letrozole, methotrexate, paclitaxel, tarceva, and trastuzumab. A chemotherapeutic agent may be administered according to any approved and/or known regimen in the art.

For example, in some embodiments, a second therapeutic agent is an anti-tumor antibody. In some embodiments, an anti-tumor antibody is an immune modulator. In some embodiments, an immune modulator is a checkpoint inhibitor. In some embodiments, a checkpoint inhibitor is an antibody or a functional fragment thereof. In some embodiments, an antibody targets one or more of PD-1, PD-L1, CTLA-4, TIM3, TIGIT, and/or LAG3. In some embodiments, an antibody targets PD-1 (e.g., pembrolizumab). An anti-tumor antibody may be administered according to any approved and/or known regimen in the art.

For example, in some embodiments, a second therapeutic agent is a surgical tumor resection. In some embodiments, a fusion polypeptide metal-hydroxide complex is administered prior to surgical tumor resection. In some embodiments, a fusion polypeptide metal-hydroxide complex is administered to tissue after tumor resection, which tissue may include, for example, remaining tumor (e.g., tumor cells). In some embodiments, a fusion polypeptide metal-hydroxide complex is administered to tissue which cannot be removed by surgical tumor resection, or tissue proximal to the resection, during said resection.

For example, in some embodiments, a second therapeutic agent is or comprises cell therapy. In some embodiments, a cell therapy is or comprises natural killer (NK) cells. In some embodiments, a cell therapy is or comprises tumor infiltrating lymphocytes (TILs). In some embodiments, a cell therapy is or comprises cells that have been expanded ex vivo. In some embodiments, a cell therapy is or comprises Chimeric Antigen Receptor (CAR) effector cell therapy (e.g., CAR T cells). CARs are genetically-engineered, artificial transmembrane receptors, which confer a selected specificity for a ligand of choice onto an immune effector cell (e.g. a T cell, natural killer cell or other immune cell) and which results in activation of the effector cell upon recognition and binding to the ligand. Often, such ligand specificity is achieved by engineering the antigen specificity of a monoclonal antibody into the CAR, thereby targeting the CAR T cell to the antigen recognized by the antibody.

In some embodiments, chimeric antigen receptor-expressing effector cells (e.g., CAR-T cells) are cells that are derived (e.g., isolated) from a patient with a disease or condition and genetically modified in vitro to express at least one CAR with an arbitrary specificity to a ligand. The cells perform at least one effector function (e.g. induction of cytokines) that is stimulated or induced by the specific binding of the ligand to the CAR and that is useful for treatment of the same patient's disease or condition. The effector cells may be T cells (e.g. cytotoxic T cells or helper T cells). One skilled in the art, reading the present disclosure, will appreciate that, in some embodiments, cells other than T cells (e.g., natural killer cells, stem cells, etc) may be engineered to express CARs, so that a chimeric antigen receptor effector cell may comprise an effector cell other than a T cell. In some embodiments, a CAR effector cell is a T cell (e.g. a cytotoxic T cell); in some embodiments, such CAR-T cell exerts its effector function (e.g. a cytotoxic T cell response) on a target cell when brought in contact or in proximity to the target or target cell (e.g. a cancer cell) (see e.g., Chang and Chen (2017) Trends Mol Med 23(5):430-450). In some embodiments, a cell therapy (e.g., a CAR effector cell therapy) utilizes of Tumor Infiltrating Lymphocytes (TILs). TILs target cancer cells. In some embodiments, TILs are isolated from a subject with cancer and expanded ex vivo. In some such embodiments, TILs are isolated and expanded ex vivo after surgical resection of the tumor. In some embodiments, before administration of TILs, a subject is treated with a lymphodepleting conditioning regimen (Rohaan, Maartje W et al. “Adoptive cellular therapies: the current landscape.” Virchows Archiv: an international journal of pathology vol. 474,4 (2019): 449-461).

In some embodiments, a cell therapy (e.g., a CAR effector cell therapy) utilizes Natural Killer (NK) cells. Natural killer (NK) cells are an essential part of tumor immunosurveillance, evidenced by higher cancer susceptibility and metastasis in association with diminished NK activity in mouse models and clinical studies. In some embodiments, for example, using an array of germline-encoded surface receptors, NK cells are able to recognize and rapidly act against malignant cells without prior sensitization (iu, S., Galat, V., Galat4, Y. et al. NK cell-based cancer immunotherapy: from basic biology to clinical development. J Hematol Oncol 14, 7 (2021)).

In some embodiments, fusion polypeptide metal-hydroxide complexes or preparations thereof as disclosed herein are administered to a subject who has received or is receiving a therapy with at least one additional therapeutic. An additional therapeutic agent may be selected from a variety of anti-tumor agents known in the art. In some embodiments, an additional therapeutic agent is administered prior to administration of a fusion polypeptide metal-hydroxide complex. In some embodiments, an additional therapeutic agent is administered concurrently with a fusion polypeptide metal-hydroxide complex. In some embodiments, an additional therapeutic agent is administered after administration with a fusion polypeptide metal-hydroxide complex.

For example, in some embodiments, an additional therapeutic is radiation (e.g., ionizing radiation). In some embodiments, an amount of ionizing radiation administered is between about 1 Gy and about 1,000 Gy, about 5 Gy and about 900 Gy, about 10 Gy to about 800 Gy, about 10 Gy to about 700 Gy, about 10 Gy to about 600 Gy, about 10 Gy to about 500 Gy, about 10 Gy to about 400 Gy, about 10 Gy to about 300 Gy, about 10 Gy to about 200 Gy, about 10 Gy to about 100 Gy, about 5 Gy and about 15 Gy, between about 7.5 Gy and about 12 Gy, or between about 10 Gy and about 12 Gy. In some embodiments, an amount of ionizing radiation administered is about 12 Gy. In some embodiments, an amount of ionizing radiation is greater than about 10, 20, 30, 40, 50, 60, 70, 80, 90, 100, 200, 300, 400, 500, or 1,000 Gy. In some embodiments, an amount of ionizing radiation is less than about 1,000, 900, 800, 700, 600, 500,400, 300, 200, 100, 90, 80, 70, 60, or 50 Gy.

For example, in some embodiments, an additional therapeutic agent is a chemotherapeutic agent. In some embodiments, an additional therapeutic agent is or comprises a targeted therapy (e.g., BRAF inhibitor, MEK inhibitor, etc.). In some embodiments, a chemotherapeutic agent may be any approved chemotherapeutic agent. For example, and without limitation, a chemotherapeutic agent can be one or more of adriamycin, anastrozole, cyclophosphamide, docetaxel, doxifluridine, doxorubicin, erlotinib, fluorouracil, gemcitabine, imatinib, iressa, letrozole, methotrexate, paclitaxel, tarceva, and trastuzumab. A chemotherapeutic agent may be administered according to any approved and/or known regimen in the art. In some embodiments, an additional therapeutic agent is an anti-tumor antibody. In some embodiments, an anti-tumor antibody is an immune modulator. In some embodiments, an immune modulator is a checkpoint inhibitor. In some embodiments, a checkpoint inhibitor is an antibody or a functional fragment thereof. In some embodiments, an antibody targets one or more of PD-1, PD-L1, CTLA-4, TIM3, TIGIT, and/or LAG3. In some embodiments, an antibody targets PD-1 (e.g., pembrolizumab). An anti-tumor antibody may be administered according to any approved and/or known regimen in the art. For example, in some embodiments an additional therapeutic agent is or comprises cell therapy. In some embodiments, a cell therapy is or comprises Chimeric Antigen Receptor (CAR) effector cell therapy (e.g., CAR T cells). CARs are genetically-engineered, artificial transmembrane receptors, which confer a selected specificity for a ligand of choice onto an immune effector cell (e.g. a T cell, natural killer cell or other immune cell) and which results in activation of the effector cell upon recognition and binding to the ligand. Often, such ligand specificity is achieved by engineering the antigen specificity of a monoclonal antibody into the CAR, thereby targeting the CAR T cell to the antigen recognized by the antibody.

In some embodiments, chimeric antigen receptor-expressing effector cells (e.g., CAR-T cells) are cells that are derived (e.g., isolated) from a patient with a disease or condition and genetically modified in vitro to express at least one CAR with an arbitrary specificity to a ligand. The cells perform at least one effector function (e.g. induction of cytokines) that is stimulated or induced by the specific binding of the ligand to the CAR and that is useful for treatment of the same patient's disease or condition. The effector cells may be T cells (e.g. cytotoxic T cells or helper T cells). One skilled in the art, reading the present disclosure, will appreciate that, in some embodiments, cells other than T cells ((e.g., natural killer cells, stem cells, etc) may be engineered to express CARs, so that a chimeric antigen receptor effector cell may comprise an effector cell other than a T cell. In some embodiments, a CAR effector cell is a T cell (e.g. a cytotoxic T cell); in some embodiments, such CAR-T cell exerts its effector function (e.g. a cytotoxic T cell response) on a target cell when brought in contact or in proximity to the target or target cell (e.g. a cancer cell) (see e.g., Chang and Chen (2017) Trends Mol Med 23(5):430-450). In some embodiments, a cell therapy (e.g., a CAR effector cell therapy) utilizes of Tumor Infiltrating Lymphocytes (TILs). TILs target cancer cells. In some embodiments, TILs are isolated from a subject with cancer and expanded ex vivo. In some such embodiments, TILs are isolated and expanded ex vivo after surgical resection of the tumor. In some embodiments, before administration of TILs, a subject is treated with a lymphodepleting conditioning regimen (Rohaan, Maartje W et al. “Adoptive cellular therapies: the current landscape.” Virchows Archiv: an international journal of pathology vol. 474,4 (2019): 449-461).

In some embodiments, a cell therapy (e.g., a CAR effector cell therapy) utilizes Natural Killer (NK) cells. Natural killer (NK) cells are an essential part of tumor immunosurveillance, evidenced by higher cancer susceptibility and metastasis in association with diminished NK activity in mouse models and clinical studies. In some embodiments, for example, using an array of germline-encoded surface receptors, NK cells are able to recognize and rapidly act against malignant cells without prior sensitization (iu, S., Galat, V., Galat4, Y. et a. NK cell-based cancer immunotherapy: from basic biology to clinical development. J Hematol Oncol 14, 7 (2021)).

In some embodiments, a cell therapy (e.g., a CAR effector cell therapy) comprises myeloid cells. In some embodiments, myeloid cells are or comprise macrophages. Macrophages have been shown to take up alum.

Table 1 provides exemplary amino acid sequences of polypeptides described herein.

TABLE 1
Exemplary Amino Acid Sequences

| SEQ ID | | |
|---|---|---|
| NO: | Polypeptide | Sequence |
| 1 | IL-12 fusion | MYRMQLLSCIALSLALVTNSIWELKKDVYVVELDWYPDAPGEMVVLTCDTPEEDGITWTLDQ |
| | polypeptide | SSEVLGSGKTLTIQVKEFGDAGQYTCHKGGEVLSHSLLLLHKKEDGIWSTDILKDQKEPKNK |
| | With 20 aa | TFLRCEAKNYSGRFTCWWLTTISTDLTFSVKSSRGSSDPQGVTCGAATLSAERVRGDNKEYE |
| | signal peptide | YSVECQEDSACPAAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVICRKNASISVRAQD |
| | | RYYSSSWSEWASVPCSGGGGSGGGGSGGGGSRNLPVATPDPGMFPCLHHSQNLLRAVSNMLQ |
| | | KARQTLEFYPCTSEEIDHEDITKDKTSTVEACLPLELTKNESCLNSRETSFITNGSCLASRK |
| | | TSFMMALCLSSIYEDLKMYQVEFKTMNAKLLMDPKRQIFLDQNMLAVIDELMQALNFNSETV |
| | | PQKSSLEEPDFYKTKIKLCILLHAFRIRAVTIDRVMSYLNASGGGGEGGGGSEEGGGGSEEG |
| | | GGGSEEGGGGSEEGGGGSEEGGGGSEEGGGGSEEGGGGSEEGGGGS |
| 2 | Mature IL-12 | IWELKKDVYVVELDWYPDAPGEMVVLTCDTPEEDGITWTLDQSSEVLGSGKTLTIQVKEFGD |
| | fusion | AGQYTCHKGGEVLSHSLLLLHKKEDGIWSTDILKDQKEPKNKTFLRCEAKNYSGRFTCWWLT |
| | polypeptide | TISTDLTFSVKSSRGSSDPQGVTCGAATLSAERVRGDNKEYEYSVECQEDSACPAAEESLPI |
| | Without 20 aa | EVMVDAVHKLKYENYTSSFFIRDIIKPDPPKNLQLKPLKNSRQVEVSWEYPDTWSTPHSYFS |
| | signal peptide | LTFCVQVQGKSKREKKDRVFTDKTSATVICRKNASISVRAQDRYYSSSWSEWASVPCSGGGG |
| | | SGGGGSGGGGSRNLPVATPDPGMFPCLHHSQNLLRAVSNMLQKARQTLEFYPCTSEEIDHED |
| | | ITKDKTSTVEACLPLELTKNESCLNSRETSFITNGSCLASRKTSFMMALCLSSIYEDLKMYQ |
| | | VEFKTMNAKLLMDPKRQIFLDQNMLAVIDELMQALNFNSETVPQKSSLEEPDFYKTKIKLCI |
| | | LLHAFRIRAVTIDRVMSYLNASGGGGEGGGGSEEGGGGSEEGGGGSEEGGGGSEEGGGGSEE |
| | | GGGGSEEGGGGSEEGGGGSEEGGGGS |
| 3 | Human IL-12B | IWELKKDVYVVELDWYPDAPGEMVVLTCDTPEEDGITWTLDQSSEVLGSGKTLTIQVKEFGD |
| | | AGQYTCHKGGEVLSHSLLLLHKKEDGIWSTDILKDQKEPKNKTFLRCEAKNYSGRFTCWWLT |
| | | TISTDLTFSVKSSRGSSDPQGVTCGAATLSAERVRGDNKEYEYSVECQEDSACPAAEESLPI |
| | | EVMVDAVHKLKYENYTSSFFIRDIIKPDPPKNLQLKPLKNSRQVEVSWEYPDTWSTPHSYFS |
| | | LTFCVQVQGKSKREKKDRVFTDKTSATVICRKNASISVRAQDRYYSSSWSEWASVPCS |
| 4 | Human IL-12A | RNLPVATPDPGMFPCLHHSQNLLRAVSNMLQKARQTLEFYPCTSEEIDHEDITKDKTSTVEA |
| | | CLPLELTKNESCLNSRETSFITNGSCLASRKTSFMMALCLSSIYEDLKMYQVEFKTMNAKLL |
| | | MDPKRQIFLDQNMLAVIDELMQALNFNSETVPQKSSLEEPDFYKTKIKLCILLHAFRIRAVT |
| | | IDRVMSYLNAS |
| 5 | IL-12 | IWELKKDVYVVELDWYPDAPGEMVVLTCDTPEEDGITWTLDQSSEVLGSGKTLTIQVKEFGD |
| | immuno- | AGQYTCHKGGEVLSHSLLLLHKKEDGIWSTDILKDQKEPKNKTFLRCEAKNYSGRFTCWWLT |
| | modulatory | TISTDLTFSVKSSRGSSDPQGVTCGAATLSAERVRGDNKEYEYSVECQEDSACPAAEESLPI |
| | polypeptide | EVMVDAVHKLKYENYTSSFFIRDIIKPDPPKNLQLKPLKNSRQVEVSWEYPDTWSTPHSYFS |
| | | LTFCVQVQGKSKREKKDRVFTDKTSATVICRKNASISVRAQDRYYSSSWSEWASVPCSGGGG |
| | | SGGGGSGGGGSRNLPVATPDPGMFPCLHHSQNLLRAVSNMLQKARQTLEFYPCTSEEIDHED |
| | | ITKDKTSTVEACLPLELTKNESCLNSRETSFITNGSCLASRKTSFMMALCLSSIYEDLKMYQ |
| | | VEFKTMNAKLLMDPKRQIFLDQNMLAVIDELMQALNFNSETVPQKSSLEEPDFYKTKIKLCI |
| | | LLHAFRIRAVTIDRVMSYLNAS |
| 6 | Metal binding | SEEGGGGSEEGGGGSEEGGGGSEEGGGGSEEGGGGSEEGGGGSEEGGGGSEEGGGGS |
| | polypeptide | |
| 7 | Human | MKMMLVRRFRVLILMVFLVACALHIALDLLPRLERRGARPSGEPGCSCAQPAAEVAAPGWAQ |
| | Fam20C-KDEL | VRGRPGEPPAASSAAGDAGWPNKHTLRILQDFSSDPSSNLSSHSLEKLPPAAEPAERALRGR |
| | | DPGALRPHDPAHRPLLRDPGPRRSESPPGPGGDASLLARLFEHPLYRVAVPPLTEEDVLFNV |
| | | NSDTRLSPKAAENPDWPHAGAEGAEFLSPGEAAVDSYPNWLKFHIGINRYELYSRHNPAIEA |
| | | LLHDLSSQRITSVAMKSGGTQLKLIMTFQNYGQALFKPMKQTREQETPPDFFYFSDYERHNA |
| | | EIAAFHLDRILDFRRVPPVAGRMVNMTKEIRDVTRDKKLWRTFFISPANNICFYGECSYYCS |
| | | TEHALCGKPDQIEGSLAAFLPDLSLAKRKTWRNPWRRSYHKRKKAEWEVDPDYCEEVKQTPP |
| | | YDSSHRILDVMDMTIFDFLMGNMDRHHYETFEKFGNETFIIHLDNGRGFGKYSHDELSILVP |
| | | LQQCCRIRKSTYLRLQLLAKEEYKLSLLMAESLRGDQVAPVLYQPHLEALDRRLRVVLKAVR |
| | | DCVERNGLHSVVDDDLDTEHRAASARGGGSKDEL |
| 11 | Mouse IL12 | MWELEKDVYVVEVDWTPDAPGETVNLTCDTPEEDDITWTSDQRHGVIGSGKTLTITVKEFLD |
| | (IL12B-linker- | AGQYTCHKGGETLSHSHLLLHKKENGIWSTEILKNFKNKTFLKCEAPNYSGRFTCSWLVQRN |
| | IL12A) | MDLKFNIKSSSSSPDSRAVTCGMASLSAEKVTLDQRDYEKYSVSCQEDVTCPTAEETLPIEL |
| | | ALEARQQNKYENYSTSFFIRDIIKPDPPKNLQMKPLKNSQVEVSWEYPDSWSTPHSYFSLKF |
| | | FVRIQRKKEKMKETEEGCNQKGAFLVEKTSTEVQCKGGNVCVQAQDRYYNSSCSKWACVPCR |
| | | VRSGGGGSGGGGSGGGGSRVIPVSGPARCLSQSRNLLKTTDDMVKTAREKLKHYSCTAEDID |
| | | HEDITRDQTSTLKTCLPLELHKNESCLATRETSSTTRGSCLPPQKTSLMMTLCLGSIYEDLK |
| | | MYQTEFQAINAALQNHNHQQIILDKGMLVAIDELMQSLNHNGETLRQKPPVGEADPYRVKMK |
| | | LCILLHAFSTRVVTINRVMGYLSSA |
| 12 | Canine IL12 | IWELEKDVYVVELDWHPDAPGEMVVLTCHTPEEDDITWTSAQSSEVLGSGKTLTIQVKEFGD |
| | (IL12B-linker- | AGQYTCHKGGKVLSRSLLLIHKKEDGIWSTDILKEQKESKNKIFLKCEAKNYSGRFTCWWLT |
| | IL12A) | AISTDLKFSVKSSRGFSDPQGVTCGAVTLSAERVRVDNRDYKKYTVECQEGSACPSAEESLP |
| | | IEVVVDAIHKLKYENYTSSFFIRDIIKPDPPTNLQLKPLKNSRHVEVSWEYPDTWSTPHSYF |
| | | SLTFCVQAQGKNNREKKDRLCVDKTSAKVVCHKDAKIRVQARDRYYSSSWSDWASVSCSGGG |
| | | GSGGGGSGGGGSRSLPTASPSPGIFQCLNHSQNLLRAVSNTLQKARQTLELYSCTSEEIDHE |
| | | DITKDKTSTVEACLPLELTMNESCLASREISLITNGSCLASGKASFMTVLCLSSIYEDLKMY |
| | | QMEFKAMNAKLLMDPKRQIFLDQNMLTAIDELLQALNFNSVTVPQKSSLEEPDFYKTKIKLC |
| | | ILLHAFRIRAVTIDRMMSYLNSS |
| 13 | Feline IL12 | IWELEKNVYVVELDWHPDAPGEMVVLTCNTPEEDDITWTSDQSSEVLGSGKTLTIQVKEFAD |
| | (IL12B-linker- | AGQYTCHKGGEVLSHSFLLIHKKEDGIWSTDILREQKESKNKIFLKCEAKNYSGRFTCWWLT |
| | IL12A) | AISTDLKFTVKSSRGSSDPQGVTCGAATLSAEKVRVDNRDYKKYTVECQEGSACPAAEESLP |
| | | IEVVVDAIHKLKYENYTSSFFIRDIIKPDPPKNLQLKPLKNSRHVEVSWEYPDTWSTPHSYF |
| | | SLTFGVQVQGKNNREKKDRLSVDKTSAKVVCHKDAKIRVQARDRYYSSSWSNWASVSCSGGG |
| | | GSGGGGSGGGGSRNLPTPTPSPGMFQCLNHSQTLLRAISNTLQKARQTLEFYSCTSEEIDHE |
| | | DITKDKTSTVEACLPLELTMNESCLASREISLITNGSCLASRKTSFMTTLCLSSIYEDLKMY |
| | | QVEFKAMNAKLLMDPKRQIFLDQNMLTAIDELLQALNVNSVTVPQNSSLEEPDFYKTKIKLC |
| | | ILLHAFRIRAVTINRMMSYLNSS |
| 14 | Porcine IL12 | IWELEKNVYVVELDWYPNAPGEMVVLTCNTPEEDGITWTSDQSSEVLGTGKTLTIHVKEFGD |
| | (IL12B-linker- | AGQYTCRKGGAVLSQSLLLLHKKEDGIWSTDILKDQKEPKNKSFLKCEAKNYSGRFTCWWLT |
| | IL12A) | AISTDLKFSVKSSRGSTDPRGVTCGTATLSEDLGEYKKYRVECQEGSACPAAEESLPIEVVL |
| | | EAVHKLKYENYTSSFFIRDIIKPDPPKNLQLNPLKNSRHVEISWEYPDTWSTPHSYFSLMFG |
| | | VQVQGKNKREKKDKLFTDQISAKVTCHKDANIRVQARDRYYSSSWSEWASVSCNGGGGSGGG |
| | | GSGGGGSRSLPATTAGPGMFKCLNHSQNLLKAVSNTLQKAKQTLEFYSCTSEEIDHEDITKD |
| | | KTSTVEACLPLELATNESCLAARETSLITNGNCLTSGKTSFMTTLCLSSIYEDLKMYHVEFQ |
| | | AMNAKLLMDPKRQIFLDQNMLTAITELMQALNFNSETVPQKPSLEELDFYKTKIKLCILLHA |
| | | FRIRAVTIDRMMSYLNSS |
| 15 | Equine IL12 | IWELEKDVYVVELDWYPDAPGEMVVLTCNTPEEEGITWTSAQSNEVLGSGKTLTIQVKEFGD |
| | (IL12B-linker- | AGWYTCHKGGEVLSHSHLLLHKKEDGIWSTDILKDQKESKNKTFLKCEAKNYSGRFTCWWLT |
| | IL12A) | AISTDLKFSVKSSRGSSDPRGVTCGAATLSAERVSVDDREYKKYTVECQEGSACPAAEESLP |
| | | IEIVVDAVHKLKYENYTSGFFIRDIIKPDPPKNLQLKPLKNSRQVEVSWEYPETWSTPHSYF |
| | | SLTFSIQVQGKNKKERKDRLFMDETSATVTCHKDGQIRVQARDRYYSSSWSEWASVSCSGGG |
| | | GSGGGGSGGGGSRNLPTATPGPGMFQCLNHSQNLLRTVSNTLQKARQTLEFYSCTSEEIDHE |
| | | DITKDKSSTVAACLPLELAPNESCLASREISFITNGSCLTPGKASSMMTLCLSSIYEDLKMY |
| | | QVEFKAMNAKLLIDPQRQIFLDENMLTAIDKLMQALNFNSETVPQKPSLEGLDFYKTKVKLC |
| | | ILLHAFRIRAVTINRMMGYLNAS |

TABLE 2
Exemplary Nucleic Acid Sequences

| SEQ ID | | |
|---|---|---|
| NO: | Nucleotide | Sequence |
| 8 | IL-12 fusion | AAGGACGTCTACGTGGTGGAACTGGATTGGTACCCAGACGCCCCTGGAGAAATG |
| | polypeptide | GTGGTGCTCACCTGTGATACCCCCGAAGAGGACGGGATTACTTGGACCCTGGAT |
| | | CAGTCATCCGAAGTGCTGGGATCCGGAAAGACCCTGACGATCCAGGTCAAGGAA |
| | | TTCGGGGACGCGGGACAGTACACTTGCCATAAGGGCGGCGAAGTGCTGTCCCAC |
| | | TCCTTGCTCCTGCTCCACAAGAAAGAAGATGGAATTTGGAGCACCGACATCCTG |
| | | AAGGACCAGAAAGAGCCGAAGAACAAGACCTTCCTGCGCTGCGAGGCCAAGAAC |
| | | TACAGCGGACGCTTTACCTGTTGGTGGCTGACCACTATCTCCACCGACCTGACC |
| | | TTCTCGGTCAAGTCCTCGCGCGGGAGCAGCGATCCTCAGGGAGTGACTTGCGGT |
| | | GCCGCGACTCTGTCGGCCGAAAGGGTCCGGGGCGACAACAAGGAGTACGAGTAC |
| | | TCAGTCGAGTGCCAGGAGGACAGCGCATGTCCGGCCGCTGAGGAATCCCTGCCT |
| | | ATTGAAGTCATGGTGGACGCCGTCCACAAGCTGAAATACGAGAACTACACCTCC |
| | | TCTTTCTTTATCCGGGATATCATCAAGCCCGACCCGCCTAAGAACCTTCAACTG |
| | | AAGCCACTGAAGAACAGCAGACAAGTGGAGGTGTCCTGGGAGTACCCGGATACT |
| | | TGGTCCACTCCGCACTCCTATTTCTCGCTCACCTTCTGCGTCCAAGTGCAGGGA |
| | | AAGTCGAAACGGGAGAAGAAGGACAGAGTGTTCACTGACAAGACCAGCGCAACT |
| | | GTGATCTGTCGGAAGAACGCCTCCATTTCCGTGCGCGCCCAGGACCGGTACTAT |
| | | TCCTCATCCTGGTCGGAGTGGGCCTCAGTGCCCTGCAGCGGCGGGGGCGGTTCG |
| | | GGTGGTGGCGGCAGCGGGGGCGGAGGGTCCCGGAATCTGCCGGTGGCGACGCCC |
| | | GACCCCGGAATGTTCCCGTGCCTGCACCATAGCCAGAACTTGCTCCGCGCGGTG |
| | | TCTAACATGCTGCAGAAGGCCCGGCAGACCCTGGAATTCTACCCCTGCACTAGT |
| | | GAAGAGATCGACCATGAAGATATTACCAAAGACAAGACTTCCACCGTGGAAGCG |
| | | TGCCTCCCACTGGAACTGACCAAGAATGAGTCCTGCCTGAATTCAAGGGAAACT |
| | | TCGTTCATTACCAACGGTTCCTGCCTGGCCTCGAGAAAGACCTCCTTCATGATG |
| | | GCTCTCTGCCTCTCCTCAATCTACGAAGATCTCAAAATGTACCAAGTGGAGTTC |
| | | AAGACCATGAACGCTAAGCTGCTGATGGACCCGAAGCGGCAGATTTTCCTCGAC |
| | | CAAAACATGTTGGCCGTGATCGACGAGTTGATGCAGGCCCTTAACTTCAACTCC |
| | | GAAACAGTGCCTCAGAAGTCGAGCCTCGAGGAACCCGACTTCTACAAGACTAAG |
| | | ATCAAGCTGTGCATCCTGCTGCACGCATTTCGAATTCGGGCCGTGACCATCGAC |
| | | CGCGTGATGAGCTACCTGAACGCATCCGGGGGCGGCGGGGAAGGAGGAGGCGGC |
| | | TCAGAGGAGGGAGGAGGAGGCAGCGAGGAAGGAGGCGGAGGAAGCGAAGAAGGA |
| | | GGGGGAGGCTCAGAAGAGGGAGGGGGTGGATCCGAGGAAGGCGGAGGAGGATCC |
| | | GAAGAGGGCGGTGGTGGATCGGAGGAAGGGGGAGGCGGTTCTGAGGAGGGTGGA |
| | | GGAGGCTCCTGA |
| 9 | Human | ATGAAGATGATGCTTGTTCGAAGATTCCGGGTCCTGATTCTGATGGTATTCCTC |
| | Fam20C-KDEL | GTCGCATGCGCCCTGCACATCGCCTTGGACCTCCTCCCTCGGCTGGAACGCAGA |
| | | GGAGCACGGCCTTCCGGAGAACCGGGCTGCTCGTGTGCACAACCAGCCGCCGAA |
| | | GTCGCTGCCCCTGGATGGGCTCAAGTCAGAGGACGGCCTGGAGAACCCCCGGCA |
| | | GCTTCCTCCGCGGCTGGAGATGCGGGATGGCCCAACAAGCACACCCTCCGGATC |
| | | CTGCAGGACTTCTCCTCCGACCCCTCCTCGAATCTGAGCTCCCACTCCCTCGAA |
| | | AAGCTGCCTCCTGCTGCCGAGCCAGCTGAACGGGCCCTGAGGGGTAGAGATCCC |
| | | GGTGCACTCAGGCCACATGACCCTGCGCACCGACCTCTGTTGAGAGATCCGGGT |
| | | CCTCGGAGGTCTGAATCACCGCCAGGTCCTGGTGGCGATGCGTCCCTTCTTGCG |
| | | CGCTTGTTCGAGCACCCGCTGTACCGGGTGGCAGTGCCTCCCCTCACCGAGGAA |
| | | GATGTGCTGTTCAACGTGAACAGCGATACCCGCCTGAGCCCGAAAGCCGCCGAG |
| | | AATCCCGACTGGCCTCATGCCGGAGCCGAAGGAGCCGAGTTCCTGAGCCCTGGA |
| | | GAAGCGGCCGTGGATTCGTACCCGAACTGGCTAAAGTTCCATATCGGCATCAAC |
| | | CGCTACGAGCTCTACTCGAGGCATAACCCGGCCATTGAGGCCCTGCTGCACGAC |
| | | CTCAGCAGCCAACGGATCACTAGCGTGGCCATGAAGTCCGGGGGCACTCAGCTG |
| | | AAGTTGATCATGACCTTTCAGAACTACGGCCAGGCCCTGTTTAAGCCCATGAAG |
| | | CAGACCCGGGAGCAGGAAACACCGCCGGACTTTTTCTACTTCTCGGACTACGAA |
| | | CGCCACAACGCCGAGATTGCCGCCTTCCACCTGGATCGCATCCTGGATTTCAGA |
| | | CGGGTGCCACCTGTGGCGGGACGCATGGTCAATATGACCAAAGAAATCCGCGAC |
| | | GTCACTCGCGACAAGAAGCTCTGGCGGACGTTCTTCATTTCCCCGGCCAACAAC |
| | | ATCTGCTTCTACGGGGAGTGCTCATACTACTGCTCCACCGAGCATGCCCTTTGC |
| | | GGAAAGCCCGACCAGATCGAAGGGTCCCTGGCAGCCTTCCTTCCCGACCTGAGC |
| | | CTGGCGAAGCGCAAGACTTGGAGAAACCCATGGCGCCGCTCATACCACAAGCGG |
| | | AAGAAGGCGGAGTGGGAGGTGGACCCCGACTACTGCGAGGAAGTCAAGCAGACC |
| | | CCCCCCTACGACTCCTCCCACCGGATTCTGGACGTGATGGACATGACTATTTTC |
| | | GATTTCCTCATGGGCAACATGGACCGGCACCACTATGAAACCTTCGAAAAGTTC |
| | | GGGAACGAGACTTTCATTATTCACCTGGACAACGGCCGGGGATTCGGAAAGTAC |
| | | AGCCACGACGAACTCTCCATCCTGGTGCCGCTGCAGCAGTGTTGCCGGATCCGC |
| | | AAGTCCACGTACCTGAGACTGCAGCTGTTGGCCAAAGAGGAGTATAAGCTGTCG |
| | | CTCCTGATGGCCGAATCCCTCCGCGGCGATCAAGTCGCCCCGGTGCTGTACCAG |
| | | CCGCATCTCGAGGCCCTGGACAGGAGACTGCGCGTGGTGCTCAAAGCTGTGCGG |
| | | GATTGTGTGGAACGGAACGGCCTGCATTCTGTGGTGGACGACGACCTGGACACC |
| | | GAACACAGAGCCGCATCCGCTAGGGGTGGAGGGTCCAAAGATGAGCTGTGA |
| 10 | IL-12 fusion | ATGTATAGAATGCAGCTTCTGTCCTGTATCGCCCTCTCCCTCGCCCTTGTGACC |
| | polypeptide | AACTCCATCTGGGAACTGAAGAAGGACGTCTACGTGGTGGAACTGGATTGGTAC |
| | (with signal | CCAGACGCCCCTGGAGAAATGGTGGTGCTCACCTGTGATACCCCCGAAGAGGAC |
| | peptide) | GGGATTACTTGGACCCTGGATCAGTCATCCGAAGTGCTGGGATCCGGAAAGACC |
| | | CTGACGATCCAGGTCAAGGAATTCGGGGACGCGGGACAGTACACTTGCCATAAG |
| | | GGCGGCGAAGTGCTGTCCCACTCCTTGCTCCTGCTCCACAAGAAAGAAGATGGA |
| | | ATTTGGAGCACCGACATCCTGAAGGACCAGAAAGAGCCGAAGAACAAGACCTTC |
| | | CTGCGCTGCGAGGCCAAGAACTACAGCGGACGCTTTACCTGTTGGTGGCTGACC |
| | | ACTATCTCCACCGACCTGACCTTCTCGGTCAAGTCCTCGCGCGGGAGCAGCGAT |
| | | CCTCAGGGAGTGACTTGCGGTGCCGCGACTCTGTCGGCCGAAAGGGTCCGGGGC |
| | | GACAACAAGGAGTACGAGTACTCAGTCGAGTGCCAGGAGGACAGCGCATGTCCG |
| | | GCCGCTGAGGAATCCCTGCCTATTGAAGTCATGGTGGACGCCGTCCACAAGCTG |
| | | AAATACGAGAACTACACCTCCTCTTTCTTTATCCGGGATATCATCAAGCCCGAC |
| | | CCGCCTAAGAACCTTCAACTGAAGCCACTGAAGAACAGCAGACAAGTGGAGGTG |
| | | TCCTGGGAGTACCCGGATACTTGGTCCACTCCGCACTCCTATTTCTCGCTCACC |
| | | TTCTGCGTCCAAGTGCAGGGAAAGTCGAAACGGGAGAAGAAGGACAGAGTGTTC |
| | | ACTGACAAGACCAGCGCAACTGTGATCTGTCGGAAGAACGCCTCCATTTCCGTG |
| | | CGCGCCCAGGACCGGTACTATTCCTCATCCTGGTCGGAGTGGGCCTCAGTGCCC |
| | | TGCAGCGGCGGGGGCGGTTCGGGTGGTGGCGGCAGCGGGGGCGGAGGGTCCCGG |
| | | AATCTGCCGGTGGCGACGCCCGACCCCGGAATGTTCCCGTGCCTGCACCATAGC |
| | | CAGAACTTGCTCCGCGCGGTGTCTAACATGCTGCAGAAGGCCCGGCAGACCCTG |
| | | GAATTCTACCCCTGCACTAGTGAAGAGATCGACCATGAAGATATTACCAAAGAC |
| | | AAGACTTCCACCGTGGAAGCGTGCCTCCCACTGGAACTGACCAAGAATGAGTCC |
| | | TGCCTGAATTCAAGGGAAACTTCGTTCATTACCAACGGTTCCTGCCTGGCCTCG |
| | | AGAAAGACCTCCTTCATGATGGCTCTCTGCCTCTCCTCAATCTACGAAGATCTC |
| | | AAAATGTACCAAGTGGAGTTCAAGACCATGAACGCTAAGCTGCTGATGGACCCG |
| | | AAGCGGCAGATTTTCCTCGACCAAAACATGTTGGCCGTGATCGACGAGTTGATG |
| | | CAGGCCCTTAACTTCAACTCCGAAACAGTGCCTCAGAAGTCGAGCCTCGAGGAA |
| | | CCCGACTTCTACAAGACTAAGATCAAGCTGTGCATCCTGCTGCACGCATTTCGA |
| | | ATTCGGGCCGTGACCATCGACCGCGTGATGAGCTACCTGAACGCATCCGGGGGC |
| | | GGCGGGGAAGGAGGAGGCGGCTCAGAGGAGGGAGGAGGAGGCAGCGAGGAAGGA |
| | | GGCGGAGGAAGCGAAGAAGGAGGGGGAGGCTCAGAAGAGGGAGGGGGTGGATCC |
| | | GAGGAAGGCGGAGGAGGATCCGAAGAGGGCGGTGGTGGATCGGAGGAAGGGGGA |
| | | GGCGGTTCTGAGGAGGGTGGAGGAGGCTCC |

### EXEMPLIFICATION

Throughout the Examples NKY-001 is used interchangeably with an IL-12 fusion polypeptide.

### Example 1: Exemplary Fusion Polypeptide Stock Preparation

This Example presents an exemplary fusion polypeptide stock preparation of a phosphorylated form of a fusion polypeptide as described herein. An exemplary amino acid sequence of interleukin-12 fusion polypeptide is shown in Table 1 and an exemplar nucleotide sequence is shown in Table 2.

Bulk purified fusion agent was supplied in 10 mM Tris, approx. 500 mM NaCl, pH 7.4 and at a concentration of approximately 11 g/L, see Table 3 and stored at 5-65° C. until formulated.

TABLE 3
Interleukin-12 fusion polypeptide bulk preparation.

| | |
|---|---|
| Bach amount | Approximately 1430 mg protein |
| Protein concentration | Approximately 11 g/L |
| Volume | 13 × 100 mL (in high density polyethylene 10 mL bottles with |
| | screw cap) |
| Buffer | 10 mM Tris, approx. 500 mM NaCl, pH 7.4 |
| Molar extinction coefficient | 1.282 mL/mg*cm |

The following chemicals and excipients were utilized (Table 4).

TABLE 4
Chemicals and excipients

| Material Name (Supplier) | Supplier Name | Material Nr (Supplier) |
|---|---|---|
| L-Histidine, USP, Multi-compendial | SAFC | H3911-1Kg |
| L-Histidine, Monohydrochloride, FCC, | Avantor ™ Performance | 2081-06 |
| Multi-compendial | Materials | |
| Trizma Base | Sigma | T1503-250g |
| Trizma Hydrochloride | Sigma | T3253-500g |
| L-Methionine | Ajinomoto | 30415 |
| Sucrose | Planstiehl | S-124-2-MC |
| Trehalose | Thermo Scientific | 309875000 |
| Sodium Chloride | Sigma-Aldrich | S7653-1Kg |
| Polysorbate 20, N.F., Multi-Compendial. | Avantor ™ Performance | 4116-02 |
| TWEEN 20 HP-LQ-(MH) | Materials | |
| Polysorbate 80, NF, Multi-compendial, | Avantor ™ Performance | 4117-02 |
| (polyoxyethylene (80) sorbitan | Materials | |
| monoleate) CRILLET 4HP | | |
| Hydrochloric Acid 1M | Merck KGaA | 1.09507.1000 |
| Sodium Hydroxide 0.1M | Merck KGaA | 1.09141.1000 |

A variety of IL-12 fusion polypeptide compositions were formulated, using different components (e.g., buffers, surfactants, excipients), and different pH conditions. Assessments of such preparations are described herein below (see Examples 2-4). Compositions were stored for a period of time—e.g., six (6) weeks or longer, and in some cases twelve (12) weeks or longer. As described below, Examples 2-4.

Each assessed composition tested in Examples 2-4 comprises IL-12 fusion polypeptide in a concentration ranging from about 0.5 mg/mL and about 3.5 mg/mL (e.g., about 2 mg/mL). Without wishing to be bound by any particular theory, a IL-12 fusion polypeptide concentration of about 2 mg/mL is a suitable concentration when later mixing the composition comprising a phosphorylated form of a fusion polypeptide with a metal-hydroxide (e.g., aluminum hydroxide) and hereby forming a fusion polypeptide metal-hydroxide complex.

### Example 2: Exemplary Fusion Polypeptide Stock Compositions

The present Example demonstrates that the degree of IL-12 fusion polypeptide stability is impacted by pH levels and/or type of buffer used in the of IL-12 fusion polypeptide composition. For example, this Example demonstrates that IL-12 fusion polypeptides are stable in a Tris buffer composition at pH around 7-8 (e.g., around 7.3-7.4). This Example also presents exemplary compositions for use in accordance with the present invention.

### Assessed Compositions

Eight different buffer/pH conditions were assessed in eight different compositions (F1-F8), see Table 5. Tris buffer and His/HisHCl buffers were evaluated in the stability assessment. NaCl concentration was 50 mM or 100 mM, if present.

TABLE 5
Composition preparations evaluated

| | Protein | | | | | | | |
|---|---|---|---|---|---|---|---|---|
| | Concentration | | Conc. | | | Conc. | Fill | |
| No. | (mg/mL) | Buffer | (mM) | pH | Excipient | (mM) | (mL) | State |
| F1 | 2 | Tris | 20 mM | 7.4 | N/A | N/A | 1.0 | Liquid |
| F2 | 2 | Tris | 20 mM | 7.4 | NaCl | 100 | 1.0 | Liquid |
| F3 | 2 | Tris | 20 mM | 7.4 | NaCl | 50 | 1.0 | Liquid |
| F4 | 2 | Tris | 20 mM | 8.0 | N/A | N/A | 1.0 | Liquid |
| F5 | 2 | His/HisHCl | 20 mM | 5.5 | N/A | N/A | 1.0 | Liquid |
| F6 | 2 | His/HisHCl | 20 mM | 6.0 | N/A | N/A | 1.0 | Liquid |
| F7 | 2 | His/HisHCl | 20 mM | 6.5 | N/A | N/A | 1.0 | Liquid |
| F8 | 2 | His/HisHCl | 20 mM | 6.5 | NaCl | 50 | 1.0 | Liquid |

Compositions (F1-F8) as described in Table 5 were prepared by buffer exchange to achieve target buffer concentration and pH. Protein concentration, osmolality, and pH of each composition was determined. All composition solutions were filtered using a 0.22 μm Polyvinylidene Fluoride (PVDF) membrane filter.

Primary packaging materials were prepared according to standard procedures, and each composition was transferred manually, observing aseptic techniques, into 2R/13 mm glass type I vials at a target fill volume of 1.0 mL, stoppered with 13 mm bromobutyl rubber stoppers (injection stoppers) and sealed with 13 mm aluminium flip-off seals. Samples of all compositions were labelled and stored at 5±3° C. till distribution for stability assessments.

Testing samples of each composition were distributed in upright position into the stability chambers according to Table 6. Vials were obtained from the different compositions at the following time points: 0 (initial/T0/frozen starting material), 1 week (T1W) and 2 weeks (T2W) at selected temperatures 5° C., 25° C. and 40° C. See Table 4 for the specific vial distribution.

TABLE 6
Vial distribution.

| | | −65° C. or | 5° C. +/− | 25° C. +/− 2° C./ | 40° C. +/− 2° C./ |
|---|---|---|---|---|---|
| Liquid | Initial | below | 3° C. | 60% +/− 5% RH | 75% +/− 5% RH |
| Initial/T0 | 1 | | | | |
| 1 week | | | 1 | 1 | 1 |
| 2 weeks | | | 1 | 1 | 1 |
| Vials/Temp | 1 | | 2 | 2 | 2 |

| | |
|---|---|
| Target vials | 7 |

At initial and subsequent time-points, testing samples of each composition were pulled and assessed as indicated below:
Visible particles (black and white) at all stability time points.
Clarity and opalescence of a solution (turbidimety) at all stability time points.
Determination of pH at all stability time points.
Osmolality by Freezing Point depression at T0/initial.
Protein content by SoloVPE at all stability time points.
Purity by Size Exclusion-HPLC at all stability time points.
Purity by reverse phase-HPLC (CR-HPLC) (reduced and non-reduced condition) at all stability time points.
CE-SDS (chip based) for F5 and F6 2 weeks at 25° C.
   Compositions after Compounding

pH, protein concentration, and osmolality of each composition after compounding and filtration were determined. Results are shown in Table 7. Protein concentration determined by UV spectrophotometer (A280) at the initial time point of the stability study is also included.
pH and protein concentration of each composition (F1-F8) were close to the desired values
Osmolality results show a wide range, as expected for each of the compositions
All compositions were colorless and free of visible particles after compounding and filtration.

TABLE 7
Results of characterization of compositions after compounding.

| | Formulations NKY-001 S27A | | | | | | | |
|---|---|---|---|---|---|---|---|---|
| Test | F1 | F2 | F3 | F4 | F5 | F6 | F7 | F8 |
| pH ^{a} | 7.3 | 7.3 | 7.3 | 7.9 | 5.6 | 6.1 | 6.6 | 6.6 |
| Osmolality by freezing | 50 | 229 | 137 | 36 | 41 | 34 | 27 | 122 |
| point [mOsm/Kg] ^{a} | | | | | | | | |
| Protein concentration | 2.0 | 1.9 | 2.0 | 2.0 | 2.1 | 1.9 | 1.9 | 2.1 |
| [mg/mL] ^{a} | | | | | | | | |

^{a} values taken over from T0 results;

### Stability Studies

No significant changes were observed over 2 weeks of the stability assessment; no visible particles were observed, pH and protein concentration were stable. Turbidity showed values between 0 and 1.

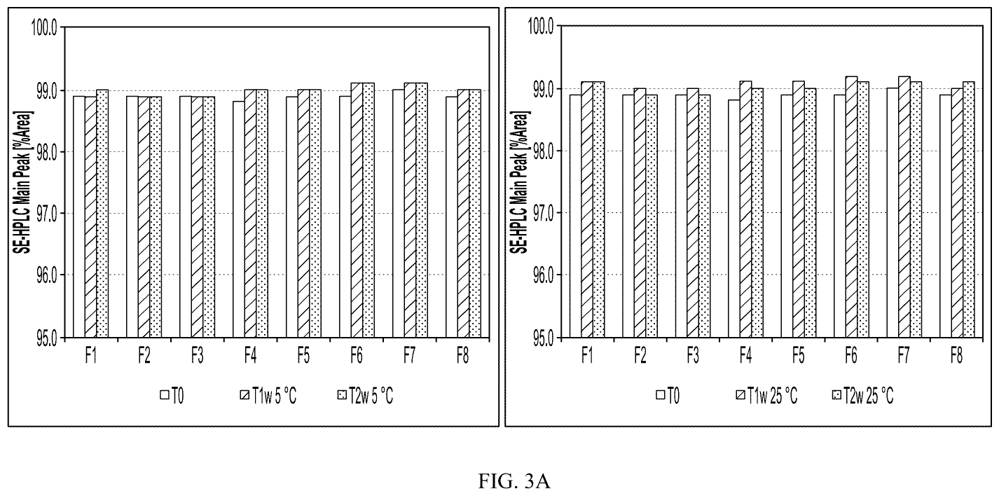

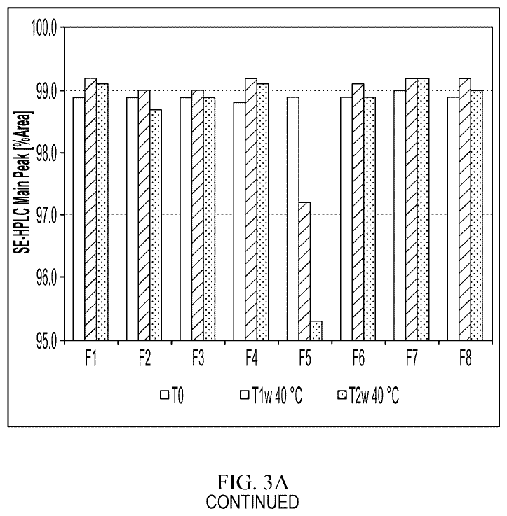

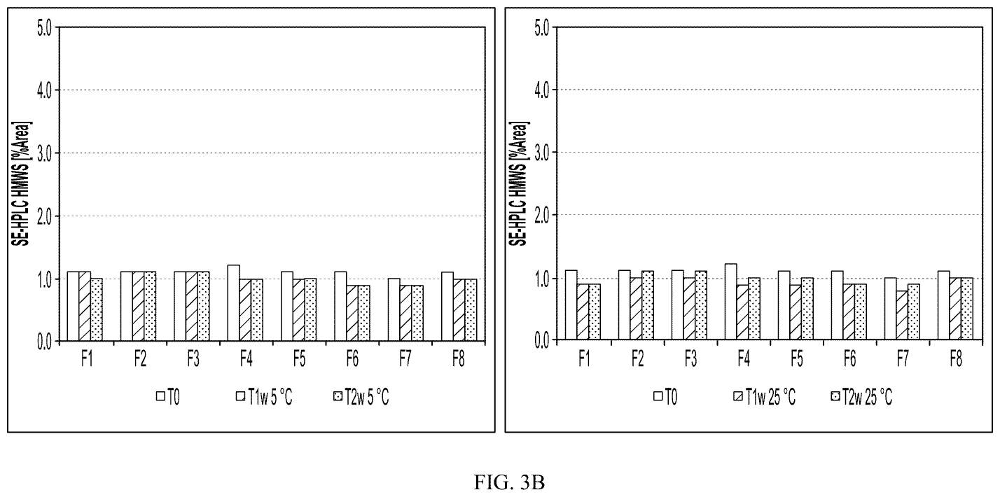

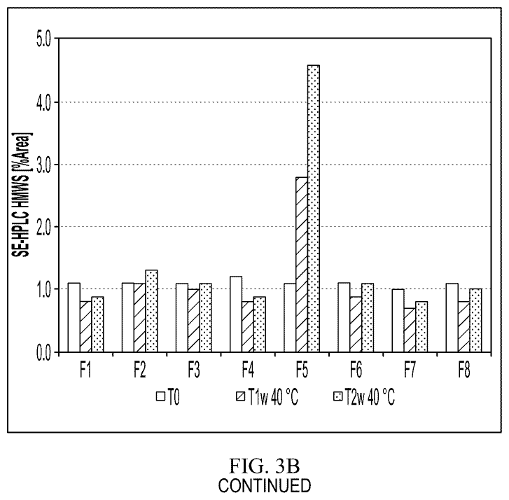

When analyzed by RP-HPLC, the composition containing Histidine buffer at lower pH (F5, pH 5.5) showed a decrease of main peak, and subsequently an increase in HMWS of about 5% at 40° C. (See FIGS. 3A-B). By contrast, compositions containing Tris buffer (F1-F4) with a higher pH, were stable over time and at the different temperatures. The present disclosure therefore demonstrates that Tris buffer can be used to effectively formulate IL-12 fusion polypeptides compositions as described herein, whereas other buffer systems (e.g., histidine buffer) may not be useful for such purpose.

More specifically, the present disclosure demonstrates that, although significant differences between or among compositions were not observed by HPLC under non-reducing conditions, under reducing conditions, some loss of the main peak was observed for the lower pH histidine buffer compositions. Specifically, F5 (at pH 5.5) showed a main peak loss above 20% and F6 (pH 6.0) showed a loss of main peak of approximately 17% after 2 weeks at 40° C., which leads to an increase of peak A (group) in both cases. Compositions F7 and F8, with a pH of 6.5 in histidine buffer, showed a more moderate decrease of main peak at 40° C. (approx. 8%). The compositions in Tris buffer (pH 7.4 and pH 8.0) showed almost no changes over time (and/or under conditions of temperature stress). Thus, the present disclosure documents surprising stability of such Tris buffer compositions.

CE-SDS (chip-based) was performed only on 2 samples: F5, F6 after 2 weeks at 25° C. Non-reduced samples did not show any differences when compared with T0 (initial/frozen starting material), i.e. 100% intact, and reduced profiles were also still very similar to T0.

Stability data up to 2 weeks showed that the interleukin-12 metal binding polypeptide fusion agent is more stable in Tris buffer at pH around 7.4, compared to Histidine buffer (evaluated at pH 5.5 to 6.5).

### Conclusion

A particularly stable composition was developed with 20 mM Tris buffer pH around 7-8 (e.g., around 7.4).

### Example 3: Impact of Surfactant on Stability

The present Example demonstrates that IL-12 fusion polypeptide compositions comprising a particular Polysorbate, Polysorbate-20, protect IL-12 fusion polypeptide against instabilities triggered by shaking stress (without Polysorbate 20 IL-12 fusion polypeptide is vulnerable to agitation). The present Example specifically establishes that Polysorbate-20 is surprisingly more effective even than another polysorbate surfactant (i.e., Polysorbate-80) at mitigating formation of visible particles upon shaking. This Example presents exemplary IL-12 fusion polypeptide compositions for use in accordance with the present invention.

### Assessed Compositions

Five different compositions were evaluated: Polysorbate 20 and 80 at two concentrations levels (0.02% (w/v) and 0.04% (w/v)), and a fifth composition without any polysorbate as a control were assessed, see the composition in Table 8.

Assessed compositions included 2 mg/ml interleukin-12 metal binding polypeptide fusion agent, 20 mM Tris buffer at pH 7.3. 50 mM NaCl was added for additional stabilization and 10 mM L-Methionine was added to mitigate possible oxidation risks.

TABLE 8
Compositions evaluated

| | Protein | | | | | | | | | |
|---|---|---|---|---|---|---|---|---|---|---|
| | conc. | | Conc. | | | Conc. | | Conc. | Fill | |
| No. | (mg/mL) | Buffer | (mM) | pH | Excipient | (mM) | Surfactant | (% w/v) | (mL) | State |
| F1 | 2 | Tris | 20 | 7.3 | NaCl L- | 50 | Polysorbate | 0.02 | 2.4 | Liquid |
| | | | mM | | methionine | 10 | 80 | | | |
| F2 | 2 | Tris | 20 | 7.3 | NaCl L- | 50 | Polysorbate | 0.04 | 2.4 | Liquid |
| | | | mM | | methionine | 10 | 80 | | | |
| F3 | 2 | Tris | 20 | 7.3 | NaCl L- | 50 | Polysorbate | 0.02 | 2.4 | Liquid |
| | | | mM | | methionine | 10 | 20 | | | |
| F4 | 2 | Tris | 20 | 7.3 | NaCl L- | 50 | Polysorbate | 0.04 | 2.4 | Liquid |
| | | | mM | | methionine | 10 | 20 | | | |
| F5 | 2 | Tris | 20 | 7.3 | NaCl L- | 50 | N/A | N/A | 2.4 | Liquid |
| | | | mM | | methionine | 10 | | | | |

Compositions as described in Table 8 were prepared by buffer exchange to achieve target buffer concentration and pH. Protein concentration, osmolality, and pH of each composition was determined. All composition solutions were filtered using a 0.22 μm Polyvinylidene Fluoride (PVDF) membrane filter.

Primary packaging materials were prepared according to standard procedures, and each composition was transferred manually, observing aseptic techniques, into 2R/13 mm glass type I vials at a target fill volume of 1.0 mL, stoppered with 13 mm bromobutyl rubber stoppers (injection stoppers) and sealed with 13 mm aluminium flip-off seals. Samples of all compositions were labelled and stored at 5±3° C. till distribution for stability assessments.

Two testing samples of each composition were subjected in horizontal position to shake stress during respectively 2 and 5 days at cool (5° C.) and room temperature (25° C.) conditions in a reciprocating (horizontal) shaker at a target speed of 200 rpm. Additionally, two testing samples of each composition were subjected in vertical position to respectively three and five freeze/thaw cycles from −65° C. or below to room temperature. See Table 9 for the specific vial distribution.

TABLE 9
Vial Distribution

| | | −65° C. or | 5° C. +/− | 25° C. +/− 2° C./ | 40° C. +/− 2° C./ |
|---|---|---|---|---|---|
| Liquid | Initial | below | 3° C. | 60% +/− 5% RH | 75% +/− 5% RH |
| Initial/T0 | 1 | | | | |
| Freeze thaw: from 25° C. | 1 | | | | |
| to −65° C., 3 cycles | | | | | |
| Freeze thaw: from 25° C. | 1 | | | | |
| to −65° C., 5 cycles | | | | | |
| 2 days shaking stress | | | 1 | 1 | |
| 5 days shaking stress | | | 1 | 1 | |

| | |
|---|---|
| Target vials | 7 |

At initial and subsequent time-points, testing samples of each composition were pulled and assessed as indicated below:
Visible particles (black and white) at all stability time points.
Clarity and opalescence of a solution (turbidimety) at all stability time points.
Colour measurement using colorimeter at all stability time points.
Sub-visible particles by Light Obscuration (LO), low volume method at all stability time points.
Determination of pH at all stability time points.
Osmolality by Freezing Point depression at all stability time points.
Protein content by SoloVPE at all stability time points.
Purity by Size Exclusion-HPLC at all stability time points.
Determination of Polysorbate 20/80 content by Florescent Micelle Assay (HPLC) at all stability time points.
Purity by reverse phase-HPLC (CR-HPLC) (reduced and non-reduced condition) at all stability time points.
CE-SDS (chip-based)
   Compositions after Compounding

pH, protein concentration, and osmolality of each composition after compounding and filtration were determined. Results are shown in Table 10. For completeness, protein concentration determined by UV spectrophotometer (A280) at the initial time point of the stability study is also included.
pH and protein concentration of each composition (F1-F5) were close to the desired values
Osmolality results show a wide range, as expected for each of the compositions
All compositions were colorless and free of visible particles after compounding and filtration.

TABLE 10
Results of characterization of compositions after compounding.

| | Formulations NKY-001 S27B | | | | |
|---|---|---|---|---|---|
| Test | F1 | F2 | F3 | F4 | F5 |
| pH^{a} | 7.3 | 7.3 | 7.3 | 7.3 | 7.3 |
| Osmolality by freezing | 145 | 144 | 144 | 145 | 144 |
| point [mOsm/Kg]^{a} | | | | | |
| Protein concentration | 2.1 | 2.1 | 2.1 | 2.1 | 2.1 |
| [mg/mL]^{a} | | | | | |

^{a}values taken over from T0 results

### Freeze-Thaw and Shaking Studies

All compositions were subjected to respectively 3 and 5 freeze-thaw cycles (−65° C. to RT), and to shaking stress at ambient temperature and at cool temperature for 2 days and 5 days.

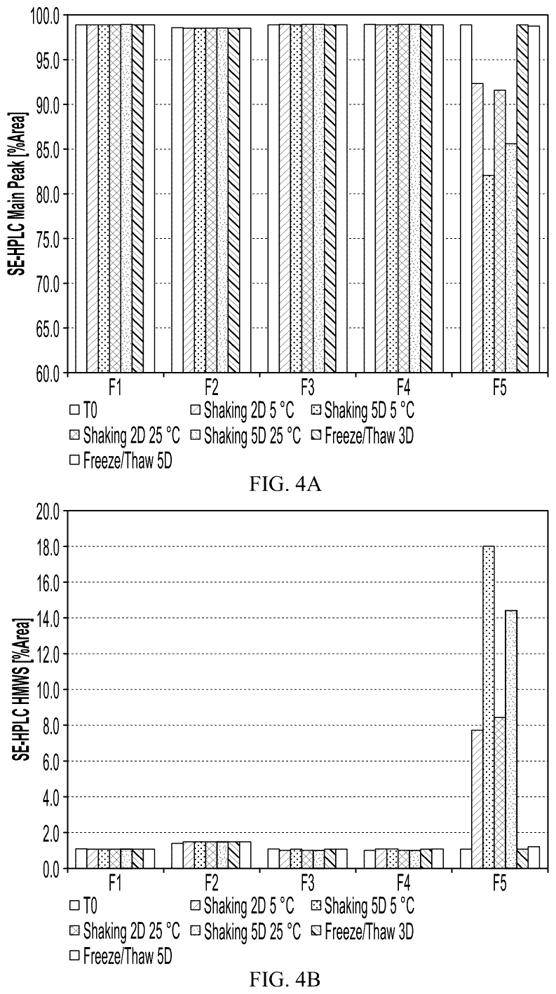

No visible particles were observed in compositions containing polysorbate (F1-F4). However, F5 showed some decrease in main peak (IL-12 fusion polypeptide) after shaking stress, which translated to an increase in HMWS (about 8% after 2 days of shaking and 15% after 5 days of shaking) (FIGS. 4A-B). Thus, many particles were observed for F5 (no polysorbate) after 5 days of shaking stress (both temperatures), and after 2 days of shaking at Room Temperature.

Freeze-thaw stress did not seem to have an impact on interleukin-12 metal binding polypeptide fusion agent for any of the compositions (F1-F5). However, F2 (PS80 0.04%) showed a slightly higher HMWS response when compared with F1 (PS80 0.02%).

No changes were observed in pH and protein concentration over the study. Turbidity and color remained stable at 1 Nephelometric Turbidity Units (NTU) and B9 respectively. For subvisible particles, some variability was observed but the values remained in a generally low range (even for the control composition without surfactant). In RP-HPLC, only small variabilities were observed in the non-reduced conditions. In reduced conditions, a minor decrease of peak A was observed for stressed samples, which was reflected in an increase main peak (approx. 0.5% to 1%). In Polysorbate content determination by generic fluorescence micelle assay (FMA), no relevant change was observed after stress conditions.

### Conclusion

Overall, Polysorbate protects interleukin-12 metal binding polypeptide fusion agent against instabilities that could be triggered by shaking stress. A higher concentration of Polysorbate (0.04%) does not seem beneficial for IL-12 fusion polypeptide compared to the lower concentration (0.02%). No real difference was observed between the two stress duration (2 days and 5 days) when polysorbate was present in the composition.

### Example 4: Impact of Other Composition Components on Stability

The present Example demonstrates that methionine has a positive impact in the stability of interleukin-12 metal binding polypeptide fusion agent, notably on preventing development of high molecular weight species (HMWS). The present Example demonstrates that IL-12 fusion polypeptide compositions assessed can be stored at 2-8° C. This Example also presents exemplary IL-12 fusion polypeptide compositions for use in accordance with the present invention.

### Assessed Compositions

Addition of methionine and sucrose vs. trehalose were tested in a composition comprising Polysorbate 20 or 80 (0.02% (w/v), see the compositions in Table 11. The surfactant assessment was performed using a composition with 2 mg/ml interleukin-12 metal binding polypeptide fusion agent, 20 mM Tris buffer at pH 7.0, 7.3 or 7.6. Buffer species, NaCl (50 mM) and L-Methionine (10 mM) were kept constant for all composition candidates. Six different compositions were tested (F1-F6).

TABLE 11
Composition compositions evaluated

| | Protein | | | | | | | | | |
|---|---|---|---|---|---|---|---|---|---|---|
| | Conc. | | Conc. | | | Conc. | | Conc. | Fill | |
| No. | (mg/mL) | Buffer | (mM) | pH | Excipient | (mM) | Surfactant | (% w/v) | (mL) | State |
| F1 | 2 | Tris | 20 | 7.3 | NaCl L- | 50 | Polysorbate | 0.02 | 2.5 | Liquid |
| | | | mM | | methionine | 10 | 20 | | | |
| | | | | | Sucrose | 150 | | | | |
| F2 | 2 | Tris | 20 | 7.3 | NaCl L- | 50 | Polysorbate | 0.04 | 2.5 | Liquid |
| | | | mM | | methionine | 10 | 80 | | | |
| | | | | | Sucrose | 150 | | | | |
| F3 | 2 | Tris | 20 | 7.0 | NaCl L- | 50 | Polysorbate | 0.02 | 2.5 | Liquid |
| | | | mM | | methionine | 10 | 20 | | | |
| | | | | | Trehalose | 150 | | | | |
| F4 | 2 | Tris | 20 | 7.6 | NaCl L- | 50 | Polysorbate | 0.02 | 2.5 | Liquid |
| | | | mM | | methionine | 10 | 20 | | | |
| | | | | | Sucrose | 150 | | | | |
| F5 | 2 | Tris | 20 | 7.3 | NaCl L- | 50 | Polysorbate | 0.02 | 2.5 | Liquid |
| | | | mM | | methionine | 10 | 80 | | | |
| | | | | | Sucrose | 150 | | | | |
| F6 | 2 | Tris | 20 | 7.3 | NaCl L- | 50 | Polysorbate | 0.02 | 2.5 | Liquid |
| | | | mM | | methionine | 10 | 20 | | | |
| | | | | | Trehalose | 150 | | | | |

Compositions as described in Table 11 were prepared by buffer exchange to achieve target buffer concentration and pH. Protein concentration, osmolality, and pH of each composition was determined. All composition solutions were filtered using a 0.22 μm Polyvinylidene Fluoride (PVDF) membrane filter.

Primary packaging materials were prepared according to standard procedures, and each composition was transferred manually, observing aseptic techniques, into 2R/13 mm glass type I vials at a target fill volume of 1.0 mL, stoppered with 13 mm bromobutyl rubber stoppers (injection stoppers) and sealed with 13 mm aluminium flip-off seals. Samples of all compositions were labelled and stored at 5±3° C. till distribution for stability assessments.

A testing sample of each composition was subjected to shake stress during approximately 5 days at room temperature in a reciprocating (horizontal) shaker at a target speed of 200 rpm. Additionally, a testing sample of each composition was subjected in vertical position to five freeze/thaw cycles from −65° C. or below to room temperature. Vials were maintained at selected temperatures −20° C., 5° C., 25° C. and 40° C., and were assessed at the following time points 0 (initial/T0/frozen starting material), 3 weeks (T3W), 6 weeks (T6W) and 12 weeks (T12W). See Table 12 for the specific vial distribution.

TABLE 12
Vial distribution

| | | | 5° C. +/− | 25° C. +/− 2° C./ | 40° C. +/− 2° C./ |
|---|---|---|---|---|---|
| Liquid | Initial | −20° C. | 3° C. | 60% +/− 5% RH | 75% +/− 5% RH |
| Initial/T0 | 1 | | | | |
| Freeze thaw: from | 1 | | | | |
| 25° C. to −65° C., 5 cycles | | | | | |
| 5 days shaking stress | | | | 1 | |
| 3 weeks | | 1 | 1 | 1 | 1 |
| 6 weeks | | 1 | 1 | 1 | 1 |
| Reserve ¹⁾ | | 1 | 1 | 1 | 1 |
| Vials/Temp | 2 | 3 | 3 | 4 | 3 |

| | |
|---|---|
| Target vials | 15 |

¹⁾ Agreement to test Reserve vials of the nominated formulation at T12wk (under Stage 27-E, SOW10)

At initial and subsequent time-points, testing samples of each compositions were pulled and assessed as indicated below:
Visible particles at all stability time points.
Clarity and opalescence of a solution (turbidimety) at all stability time points.
Color measurement using colorimeter at all stability time points.
Sub-visible particles by Light Obscuration (LO), low volume method at all stability time points.
Determination of pH at all stability time points.
Osmolality by Freezing Point depression at all stability time points.
Protein content by SoloVPE at all stability time points.
Purity by Size Exclusion-HPLC at all stability time points.
Purity by Anion Exchange Chromatography (AEX) at all stability time points, except FT (Freeze/Thaw) and shaking.
Determination of Polysorbate 20/80 content by Fluorescent Micelle Assay (HPLC) at all stability time points.
Purity by reverse phase-HPLC (CR-HPLC) (reduced and non-reduced condition) at all stability time points.

Furthermore, 200 μl aliquot for Alum-binding assay 100 μl aliquot for Potency assay and 100 μl aliquot for ELISA were taken for each composition at baseline (stored at −65° C.).

Compositions after Compounding

pH, protein concentration, and osmolality of each composition after compounding and filtration were determined. Results are shown in Table 13. For completeness, protein concentration determined by UV spectrophotometer (A280) at the initial time point of the stability study is also included.
pH and protein concentration of each composition (F1-F5) were close to the desired values
Osmolality results show a wide range, as expected for each of the compositions
All compositions were colorless and free of visible particles after compounding and filtration.

TABLE 13
Results of characterization of compositions after compounding.

| | Formulations NKY-001 S27C | | | | | |
|---|---|---|---|---|---|---|
| Test | F1 | F2 | F3 | F4 | F5 | F6 |
| pH^{a} | 7.2 | 7.2 | 6.9 | 7.5 | 7.3 | 7.3 |
| Osmolality by freezing | 314 | 311 | 313 | 310 | 301 | 311 |
| point [mOsm/Kg]^{a} | | | | | | |
| Protein concentration | 2.1 | 2.0 | 2.0 | 2.0 | 2.1 | 2.1 |
| [mg/mL]^{a} | | | | | | |

^{a}values taken over from T0 results

### Freeze-Thaw and Shaking Studies

All compositions, subjected to 5 freeze-thaw cycles (−65° C. to RT) or shaking stress at ambient temperature did not show any relevant changes in any of the analytical methods compared to the initial non-stressed samples, indicating that compositions effectively stabilized the IL-12 polypeptide fusion agent against both freeze-thaw and shaking stress. Only F1 and F5 seemed to have slightly higher particle counts after 5 days of shaking stress.

### Short-Term Stability Studies (Thermal Stress): Storage at −20° C., 5° C., 25° C., 40° C.

No visible particles were observed for any of the compositions, turbidity was stable (0 to 1 NTU) and color was ranging from colorless to B9. Protein concentration and pH remained stable over time.

Subvisible particles were generally low in number. Subvisible particles 225 μm were detected in very few of the samples (and only as 1 or 2 particles).

No major differences were observed over time in percentage of main peak at temperatures up to 25° C. for all compositions analyzed using the SEC method. After 6 weeks at 40° C., some LMWS appeared (50.1%) and HMWS showed an increase by approx. 0.3% compared to T0 for F1, and up to 0.5% for F4 and F5.

In RP-HPLC, the reduced condition seemed more stability indicating than the non-reduced condition. Some signs of degradation were observed at 40° C., especially for F5 (without L-methionine) (5% main peak loss after 6 weeks), which indicates that methionine is stabilizing the fusion polypeptide.

In Polysorbate content determination, only small variabilities were observed for samples containing PS20. Samples containing PS80 (F2 and F5) showed some degradation at 40° C.

The T12W time point was pulled for the nominated composition (FR) to decide the intended storage condition (refrigerated vs frozen −20° C.).

Long term (12 weeks time point) stability studies for nominated composition (F1)

Analysis of one additional time point was performed only for the nominated composition (F1), in order to support a decision regarding the storage condition.

After 12 weeks, no visible particles were observed at any storage temperature. No differences were observed in turbidity (remained at 1 NTU), color, pH and protein concentration. Less than or equal to 25 subvisible particle having a diameter of more than or equal to 10 μm per mL were observed in the compositions and less than or equal to 3 subvisible particle having a diameter of more than or equal to 10 μm per mL were observed in the compositions

By SEC, only the 40° C. sample showed some decrease in main peak (1%), which was reflected in an increase of HMWS (by approx. 1.0-1.2% compared to T0 and frozen analytical reference), and apparition of 0.1% LWMS.

The RP-HPLC non-reduced did not show any relevant changes for the compositions. In reduced conditions, a decrease of main peak (approx. 10-13%) was observed for the 40° C. storage, which is reflected in a corresponding increase in Peak A (by approx. 10-11%).

No decrease of polysorbate content was observed in any storage condition.

Overall, no differences were observed between 5° C. and −20° C. storage conditions for all purity methods; the potential difference in SVP counts may be related to method variability which is inherent to particle counting methods.

In conclusion, interleukin-12 metal binding polypeptide fusion agent compositions tested can be stored at 2-8° C.

### Sample Analysis by Anion Exchange Chromatography (AEX) Development Method

The method utilizes a digestion with a phosphatase kit during sample preparation, prior to analysis by HPLC. FT and shaking stress were not analyzed by AEX. Fomposition samples from the short-term stability study had been frozen after pull and were analyzed together around the time of the 12 wk pull for F1, in sequences per time point and including frozen analytical control. AEX was run as duplicate injections, and some variability was observed.

During implementation runs, profiles obtained in DPS looked similar to profiles known from assay development, with two peaks of similar height separated by a valley. Results for one main peak and the second (earlier) peak labelled acidic peak 1 were reported. This profile was generally maintained over the course of the formulation study, with only minor differences between compositions. Samples stored at −20° C., 5° C. and 25° C. resulted in similar profiles, potentially showing a slight shift in peak height towards the earlier peak (e.g. 6 weeks at 25° C.). In contrast, the samples stored at 40° C. started showing a loss of peak resolution at 3 weeks in all compositions (still two peaks but with a much less defined valley), and at 6 weeks, the signal could not be integrated as two separate peaks anymore; a distinction between “main” and “acidic” peaks was not possible since in some samples, the highest signal occurred at a retention time between the peak assignment.

It was noted that for F1 analyzed at 12 weeks, the 25° C. sample showed some loss of definition of the valley between the two peaks. The 40° C. sample showed a profile change towards a pre-peak shoulder and a more defined peak with a retention time similar to what had been assigned as main peak during method development.

### Conclusion

Temperature can impact the compositions, as is particularly observed in the reduction of primary peaks on RP-HPLC at 40° C. Inclusion of methionine improves stability of interleukin-12 metal binding polypeptide fusion agent, notably on development of HMWS. All pHs within the tested ranges were acceptable. Polysorbate 20 was confirmed to be more suitable for IL-12 fusion polypeptide than Polysorbate 80 (especially at 40° C.). Provided compositions were demonstrated to be stable even to freeze-Thaw and shaking stress, which indicates adequate behavior for handling during manufacturing.

### Example 5: Aluminum Hydroxide (Alum) Retention Assay

This Example presents an exemplary pharmaceutical composition comprising a fusion polypeptide metal-hydroxide complex. The present Example demonstrates that such pharmaceutical composition is stable and that aluminum hydroxide complexation does not affect fusion polypeptide stability.

Binding and retention of IL-12 fusion polypeptide to aluminum hydroxide was tested in vitro. An human IL-12 fusion polypeptide composition (20 mM Tris, 150 mM sucrose, 50 mM NaCl, 10 mM L-Methionine, 0.02% w/v Polysorbate 20, at pH 7.3) was mixed with aluminum hydroxide (Invivogen Cat #alu-vac-250) to a final concentration of 250 μg/mL IL-12 fusion polypeptide and 2.5 mg/mL aluminum hydroxide or composition buffer only as a control to a final volume of 40 μL.

IL-12 fusion polypeptide/alum mixtures were resuspended thoroughly by pipetting and incubated at room temperature for 30 minutes. The IL-12 fusion polypeptide/alum mixtures or IL-12 fusion polypeptide only controls were then diluted 25× in elution buffer containing a final concentration of 1 mM phosphate, 40% human serum to a final volume of 1 mL. Diluted samples were incubated at 37° C. with gentle rotating for 2-24 hours. At each timepoint, 50 μL of sample was removed and centrifuged at 18,000×g for 10 minutes to pellet the aluminum hydroxide. Cleared supernatant was transferred to a new tube and stored at 4C until ready for analysis. The concentration of free IL-12 fusion polypeptide in each supernatant sample was quantified using a human IL12p70 ELISA with R&D Systems MAB219 as the capture reagent and Biolegend antibody 508802 as the detection reagent. All dilutions were made in TBS+1% BSA+0.1% Tween-20. Test agents were used for standard curves with a top concentration of 0.5 ng/mL and 2× dilutions and supernatant samples were diluted to a theoretical concentration of 0.25 ng/mL if all polypeptide was released.

Protein stocks AK346B, IL-12 fusion polypeptide complexed to alum_B1 or IL-12 fusion polypeptide complexed to alum_B2: 0.333 mg/ml in TBS or F1. F1 is an exemplary fusion polypeptide composing comprising 20 mM Tris, 150 mM sucrose, 50 mM NaCl, 10 mM L-Methionine, 0.02% w/v Polysorbate 20, at pH 7.3.

All alum samples: 10 mg/mL (neat)

A-H described below were eluted in TBS/PBS with 1 mM phosphate 40% human serum. PBS: 1×PBS (pH 7.4; 11.8 mM PO₄) via CSH protocol; 20 mM TBS (pH 7.4). Human Serum Gender Pooled; BioVT, Cat #HUMANSERM-0001255, Lot #: HMN749277. Timepoints: 2 h and 24 h incubation at 37° C. B1 and B2 are two different preparations of purified IL-12 fusion polypeptide.

### Experimental Conditions, Proteins and Concentrations

A: 10 μg/mL AK346B (TBS); 30 μL protein+10 μL TBS—no alum control
B: 10 μg/mL ANK101_B1 (F1); 30 μL protein+10 μL F1 buffer—no alum control
C: 10 μg/mL ANK101_B2 (F1); 30 μL protein+10 μL TBS—no alum control
D: 10 μg/mL AK346B (TBS); 30 μL protein+10 μL alum—(10:1 alum: protein ratio)
E: 10 μg/mL ANK101_B1 (F1); 30 μL protein+10 μL alum'(10:1 alum: protein ratio)
F: 10 μg/mL ANK101_B2 (F1); 30 μL protein+10 μL alum—(10:1 alum: protein ratio)
G: 10 μg/mL ANK101_B1 (TBS); 30 μL protein+10 μL alum—(10:1 alum: protein ratio)
H: 10 μg/mL ANK101_B2 (TBS); 30 μL protein+10 μL alum—(10:1 alum: protein ratio)

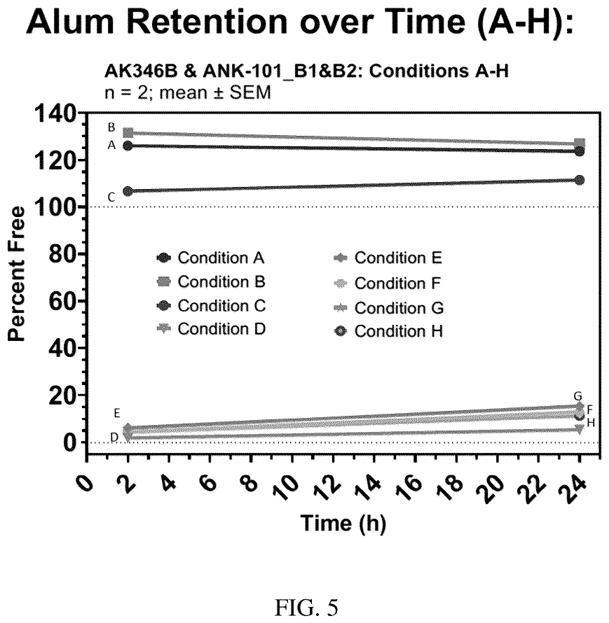

IL-12 fusion polypeptide in formulation buffer forms a complex with aluminum hydroxide that remains stable after incubation in 1 mM phosphate and 40% human serum. Only ^{˜}4% of protein was released at 2 hours and ^{˜}14% at 24 hours (FIG. 5, conditions E/F), which is highly similar to release of IL-12 fusion polypeptide when formulated in TBS prior to aluminum hydroxide complexation (FIG. 5, conditions G/H).

### Example 6: IL-12 Signaling Activity Assay

The present Example demonstrates that the IL-12 fusion polypeptide retain its biological activity when formulated in a pharmaceutical composition according to the present disclosure.

In vitro IL12 signaling activity was assessed using the Promega IL12 Bioassay kit (JA2601) according to manufacturer's instructions. The IL12 Bioassay uses human cells engineered to express the IL12 receptor and a luciferase reporter under the control of an IL12 inducible promoter. Promega IL12 reporter cells are supplied in a frozen, ready to use format that does not require cell culture.

IL-12 fusion polypeptide formulated in TBS or optimized IL-12 fusion polypeptide composition (20 mM Tris, 150 mM sucrose, 50 mM NaCl, 10 mM L-Methionine, 0.02% w/v Polysorbate 20, at pH 7.3) were diluted in assay media to generate a titration series with a top concentration of 3 μg/mL and 3× dilutions. For samples mixed with alum, fusion polypeptides at a final concentration of 250 μg/mL were mixed with a 10× mass excess of aluminum hydroxide as defined by metal mass in formulation buffer and incubated at room temperature for 30 minutes with shaking before diluting in assay media as above. 25 μL of each sample in the titration series was transferred to a 96 well plate and mixed with 50 μL of Promega cell suspension for a final top fusion polypeptide concentration of 1 μg/mL. Plates were then incubated overnight at 370C in 5% CO2 for 6 hours. 75 μL of Bio-Glo reagent was added to sample wells, incubated for 10 minutes, and luminescence measured.

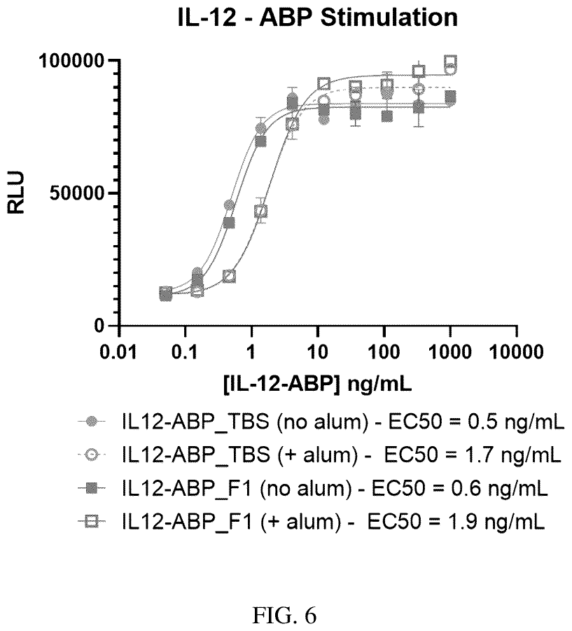

The IL12 fusion polypeptide in an optimized IL12 fusion polypeptide composition induces potent signaling in the Promega IL12 reporter assay both alone and after Alhydrogel complexation (FIG. 6). The EC50 values are highly similar to IL12 fusion polypeptide in TBS suggesting that the formulation does not impact biological activity.

### Example 7: Impact of Other Composition Components on IL-12 Fusion Polypeptide Stability

The present Example demonstrates that IL-12 fusion polypeptide when formulated in a composition comprising Tris buffer, sucrose, a salt, L-methionine, and a surfactant with a pH of about 7-7.5 exhibit low oxidation and low deamidation. In particular this example demonstrates that L-methionine in the composition protects against oxidation of methionine and tryptophan in the IL-12 fusion polypeptide and a low pH (about 7-7.5) protects against deamidation of asparagine and glutamine.

Composition F1, F2, F4 and F5 were prepared as described in Example 4. Compositions were incubated for 6 weeks at 40° C.

Oxidation (%) of methionine and tryptophan is shown in Table 14 below.

TABLE 14
Oxidation of Methionine and Tryptophan [%]

| | | | W2324, | | | | | | | | |
|---|---|---|---|---|---|---|---|---|---|---|---|
| | W90 | M189 | W240 | M333 | M350 | M418 | M419 | M432 | M446 | M458 | M466 |
| F1 T0 | 0.2 | 1.0 | n.d. | 6.5 | 2.0 | 2.5 | 0.6 | 2.9 | 3.0 | 1.0 | 0.8 |
| CTRL | | | | | | | | | | | |
| F1 | 0.2 | 0.9 | 0.4 | 5.6 | 1.9 | 2.5 | 0.6 | 2.2 | 3.0 | 0.8 | 0.6 |
| T6w | | | | | | | | | | | |
| 40° C. | | | | | | | | | | | |
| F2 | 0.3 | 1.0 | n.d. | 5.2 | 2.1 | 2.7 | 0.5 | 2.4 | 3.8 | 0.7 | 0.5 |
| T6w | | | | | | | | | | | |
| 40° C. | | | | | | | | | | | |
| F4 | 0.3 | 0.9 | n.d. | 4.7 | 1.9 | 2.6 | 0.5 | 2.3 | 3.8 | 0.2 | 0.4 |
| T6w | | | | | | | | | | | |
| 40° C. | | | | | | | | | | | |
| F5 | 0.3 | 1.4 | n.d. | 7.9 | 2.2 | 4.9 | 0.8 | 2.9 | 5.1 | 0.8 | 0.9 |
| T6w | | | | | | | | | | | |
| 40° C. | | | | | | | | | | | |

Composition F5 (without Methionine) exhibited 27% oxidized species vs. F1, F2 and F4 (all with Methionine) exhibited ^{˜}18-20% oxidized species. Showing that L-methionine is capable of reducing or preventing oxidation of methionine and tryptophan in composition F1, F2 and F4.

Deamination (%) of Asparagine (N) and Glutamine (Q) is shown in Table 15 below.

TABLE 15
Deamidation of Asparagine and Glutamine [%]

| | Q56 | Q65 | Q144 | N162 | N218 | Q220 | Q254 |
|---|---|---|---|---|---|---|---|
| F1 T0 CTRL | 0.3 | n.d. | 0.2 | n.d. | 0.1 | 0.2 | 0.9 |
| F1 T6w 40° C. | 0.3 | 0.2 | 0.3 | 9.0 | 0.1 | 0.2 | 0.4 |
| F2 T6w 40° C. | 0.3 | 0.2 | 0.3 | 9.9 | <0.1 | 0.2 | 0.9 |
| F4 T6w 40° C. | 0.3 | 0.2 | 0.3 | 11.1 | 0.1 | 0.1 | 1.0 |
| F5 T6w 40° C. | 0.4 | 0.2 | 0.3 | 9.3 | 0.1 | 0.3 | n.d. |

| | N323 | N349 | Q352 | Q434 | N441 | Q456 | Q467 | N472 |
|---|---|---|---|---|---|---|---|---|
| F1 T0 CTRL | 0.2 | 0.1 | 0.4 | 0.1 | n.d. | <0.1 | n.d. | 3.8 |
| F1 T6w 40° C. | 1.0 | 0.2 | 0.4 | 0.1 | 0.1 | n.d. | n.d. | 18.4 |
| F2 T6w 40° C. | 0.9 | 0.1 | 0.4 | 0.1 | 0.1 | n.d. | 0.5 | 16.2 |
| F4 T6w 40° C. | 1.6 | 0.2 | 0.4 | 0.1 | n.d. | 0.2 | <0.1 | 24.1 |
| F5 T6w 40° C. | 1.1 | 0.1 | 0.3 | 0.2 | n.d. | n.d. | <0.1 | 18.4 |

n.d.: no modification detected.
No modification detected for other positions.

Table 15 depicts the effects of pH on deamidation. Composition (F4) having the highest pH (pH 7.6) exhibited significantly higher levels of deamidation for N162, N323, and N472 compared to compositions having a lower pH (F1, F2, and F5 having a pH of about 7.3).

### Example 8: Formulation Development of IL-12 Fusion Polypeptide Alone or Complexed with Aluminum Hydroxide

The present Example demonstrates that IL-12 fusion polypeptide drug product can be complexed to aluminum hydroxide (alum). The Example also demonstrates that IL-12 fusion polypeptide complexed to alum does not significantly impact IL-12 fusion polypeptide potency or PBMC viability. Preparation of complexed IL-12 fusion polypeptide using syringes does not result in loss of IL-12 fusion polypeptides.

### Exemplary IL-12 Fusion Polypeptide Drug Products

IL-12 fusion polypeptide drug product is made available as 1.5 mg/vial, manufactured directly from the fully formulated drug substance. The IL-12 fusion polypeptide drug product is a sterile formulation contained in a single use vial, each vial nominally containing 1.5 mg.

The IL-12 fusion polypeptide drug substance component (nominal concentration 2 mg/mL) is fully formulated at the drug substance stage in 20 mM Tris, 50 mM Sodium chloride, 150 mM Sucrose, 0.02% Polysorbate 20 (w/v), 10 mM L-Methionine, at a target pH of 7.3. IL-12 fusion polypeptide drug substance is the only active ingredient in the drug product (IL-12 fusion polypeptide drug product 1.5 mg/vial).

The qualitative and quantitative composition of the IL-12 fusion polypeptide drug product is the same as the IL-12 fusion polypeptide drug substance. No incompatibility exists between the excipients and the active substance, as shown in the stability studies for both drug substance and drug product.

IL-12 fusion polypeptide drug product was developed for intratumoral administration for clinical trials. IL-12 fusion polypeptide drug product is composed of 1.5 mg IL-12 fusion polypeptide/vial in glass vials.

Early-stage formulation development studies, including agitation, freeze/thaw and storage stability studies have confirmed the suitability of this formulation and dosage form. The buffer and pH were selected to provide a stable solution for the protein while maintaining pH during storage for drug substance and drug product. Polysorbate 20 has been added to reduce the potential for agitation and/or freeze/thaw induced aggregation. Sucrose has been added to adjust the osmolality of the product. The formulation is designed to be robust with respect to freeze/thaw cycles.

### Administration Components and Simulated Use

A simulated administration study was performed to assess the initial step in the dosage preparation, including compatibility of the product with several components and contact materials expected to be used during the dose preparation for clinical drug administration via intratumoral route.

The compatibility of the diluted IL-12 fusion polypeptide drug product with the product-specific diluent was studied in a type 1 glass vial (6R) to establish the stability of the diluted drug product and qualify representative clinical dosage preparation materials. The IL-12 fusion polypeptide drug product (Lot 101) was presented as a liquid in the following formulation: 20 mM Tris, 150 mM Sucrose, 50 mM Sodium chloride, 10 mM L-Methionine, 0.02% (w/v) Polysorbate 20, pH 7.3, at a nominal concentration of 2 mg/mL (representative batch with nominal fill volume was 1.0 mL in 2R glass vials [Type 1] including overfill). The product-specific diluent was presented as a liquid in the following formulation: 20 mM Tris, 150 mM Sucrose, 50 mM Sodium chloride, 10 mM L-Methionine, 0.02% (w/v) Polysorbate 20, pH 7.3 (representative batch with nominal fill volume of 6 mL in 6R glass vials [Type 1]). IL-12 fusion polypeptide drug product and the product-specific diluent were transferred into sealed empty sterile 6R glass vials using commercially available siliconized syringes (1 mL or 2 mL) and needles (21 gauge).

The IL-12 fusion polypeptide drug product was diluted with product-specific diluent solution into 6R vials to target concentration of 0.25 mg/mL. The diluted drug product (at 0.25 mg/mL) was prepared in triplicate (n=3). The physicochemical stability of diluted IL-12 fusion polypeptide drug product solution in 6R sterile sealed vials for up to 4 hours with exposure to ambient storage conditions (ambient temperature with exposure to light) is supported by physicochemical analytical data.

Throughout the study, no major changes were observed in the physicochemical analytical tests (clarity, color), purity by size exclusion-high performance liquid chromatography, and activity indicating good compatibility with the selected materials. The time 0 samples tested were practically free of visible particles (1 of 3 replicates contained 1 fiber-like particle). For the time at 4 hours (T4 h), a reported result of few visible particles was assigned for the samples (2 of 3 replicas contained 1 fiber-like particle each). In further investigation by particle characterization, these visible particles were reported as non-proteinaceous, mainly cellulose fibers and some oleamide particles. Those visible particles were therefore inherent to the dosage preparation. Considering the expected administration volume below 100 mL, subvisible particles were well within the pharmacopeia requirements <United States Pharmacopoeia 787> in both subvisible particle counts for ≥25 μm (≤600 counts/container) and ≥10 μm (5 6,000 counts/container).

Recoveries at T4h were overall high for all samples (all above 99%), showing good compatibility with the contact materials.

Cell-based Assay results for IL-12 fusion polypeptide drug product at T0 and T4 showed 102% activity (T0) and 103% activity (T4).

These results show that IL-12 fusion polypeptide drug product is stable in the product-specific diluent at the target concentration (0.25 mg/mL) in contact with glass vials for up to 4 hours at ambient conditions.

Potency and Strength of IL-12 Fusion Polypeptide Complexed with Alhydrogel® at Dose Preparation

Aluminum hydroxide (alum) that is the chemical basis of Alhydrogel®.

The compatibility of the complexed IL-12 fusion polypeptide drug product/Alhydrogel® was interrogated with an in-use compatibility study that emulated drug preparation at the clinical pharmacy utilizing IL-12 fusion polypeptide drug product or drug substance, Alhydrogel®, Diluent, sterile empty vials (SEVs), and commonly available ancillary components (e.g., syringes, needles). A bracketing design was employed to cover the intended dose range including an intermediate dose. Dose preparations are described below:

High Dose: 0.25 mg/mL IL-12 fusion polypeptide Complexed with 2.5 mg of Alhydrogel® (Dose Group 6)
1) 3.75 mL of Diluent drug product was added into a 6R SEV.
2) 0.75 mL of IL-12 fusion polypeptide drug product/drug substance (2 mg/mL) was added into the diluent containing 6R vial and gently swirled to ensure mixing.
3) The vial of Alhydrogel® was shaken well to ensure homogeneity.
4) 1.5 mL of Alhydrogel® (10 mg/mL) was added into the diluted solution of IL-12 fusion polypeptide drug product and gently swirled to ensure mixing.
   a. The concentration of IL-12 fusion polypeptide drug product/drug substance=0.250 mg/mL
   b. The concentration of Alhydrogel®=2.5 mg/mL
   c. A total volume of IL-12 fusion polypeptide, 6 mL, was contained within the 6R vial
5) The mixed preparation was incubated for 30 minutes or 6 hours at room temperature.

Middle Dose: 0.02 mg/mL IL-12 fusion polypeptide Complexed with 0.2 mg of Alhydrogel® (Dose Group 3)

Starting with the previously prepared solution of 0.250 mg/mL IL-12-fusion polypeptide/2.5 mg/mL Alhydrogel® (Dose Group 6) from the initial mixing process described above, the following dilution scheme was performed:
1) The vial prepared for Dose Group 6 was mixed gently.
2) 5.52 mL of Diluent drug product was added into a 6R SEV.
3) 0.48 mL of Dose Group 6 was added into the diluent containing 6R vial and gently swirled to ensure mixing.
   a. The concentration of IL-12 fusion polypeptide drug product/substance=0.02 mg/mL.
   b. The concentration of Alhydrogel®=0.2 mg/mL
4) The mixed preparation was incubated for 30 minutes or 6 hours at room temperature.

Low Dose: 0.002 mg/mL IL-12-ABP Complexed with 0.02 mg of Alhydrogel® (Dose Group 1).

Starting with the previously prepared solution of 0.02 mg/mL IL-12 fusion polypeptide/0.2 mg/mL Alhydrogel® (Dose Group 3) from the mixing process described above, the following dilution scheme was performed:
1) The vial prepared for Dose Group 3 was mixed gently.
2) 5.4 mL of Diluent drug product was added into a 6R SEV.
3) 0.6 mL of Dose Group 3 was added into the diluent containing 6R vial and gently swirled to ensure mixing.
   a. The concentration of IL-12 fusion polypeptide drug product/substance=0.002 mg/mL.
   b. The concentration of Alhydrogel®=0.02 mg/mL.
4) The mixed preparation was incubated for 30 minutes or 6 hours at room temperature.

Interleukin-12 (IL-12) signals through a heterodimeric complex of IL-12Rβ1 and IL-12Rβ2 expressed on T and natural killer (NK) cells to induce interferon gamma (IFNγ) expression through phosphorylation and activation of STAT4. The potency of free IL-12-ABP protein and complexed IL-12 fusion polypeptide to induce IFNγ expression from activated primary human peripheral blood mononuclear cells (PBMCs) from healthy donors was assessed in comparison to a human IL-12 control protein lacking the alum-binding protein. Cells were treated with a titration of human IL-12 (control), IL-12 fusion polypeptide, or IL-12 fusion polypeptide complexed to alum in the presence of 100 ng/mL soluble α-cluster of differentiation (CD) 3 antibody (clone OKT3) for PBMCs. After 3 days, IFNγ concentration in the supernatant was measured by time-resolved fluorescence energy transfer (TR-FRET) assay. The concentrations of a-CD3 antibody were selected to suboptimally activate the immune cells and increase their responsiveness to the IL-12 agents while inducing minimal IFNγ on their own.

In this study, human PBMCs were isolated from 2 healthy donors and seeded at 5×10⁵ cells per well in round bottom 96-well plates. PBMCs were stimulated with anti-CD3 (100 ng/mL) in the presence of IL-12 fusion polypeptide drug substance (Good Manufacturing Practice Lot 1205114). IL-12 fusion polypeptide was prepared via either a syringe (at all steps or for extraction from the dose vial only) or prepared by using a pipette throughout (Study ATXFTE-06). Appropriate controls included negative control (unstimulated PBMCs), positive control (soluble CD3 [5 μg/mL]+soluble CD28 [2 μg/mL]), and Diluent (formulation buffer). Following a 72-hour incubation, cell culture supernatants were harvested and stored at −80° C. until cytokine analysis by TR-FRET for IFNγ was completed. Cell viability was determined using the CellTiter-Glo® 2.0 Cell Viability Assay. The study was performed utilizing PBMCs from 2 donors. Each experimental condition was tested in triplicate, and each immune assay readout was performed in singlicate (Table 16).

TABLE 16
PBMC Assay Groups Study Summary

| Complex Preparation/Control | | | | | | |
|---|---|---|---|---|---|---|
| Condition | | | | | | |
| | | Incubation | | | | |
| Method | Dose | Time | | Stimulation | Final Dose | Readouts |
| Unstimulated PBMC | | | | NA | NA | Day 3 |
| Positive Control | | | | anti-CD3 (5 μg/mL) + | NA | Cell viability: |
| | | | | anti-CD28 (2 | | CellTiter-Glo ® |
| | | | | μg/mL) | | 2.0 Cell Viability |
| Vehicle (Formulation Buffer) | | | | anti-CD3 (100 | 0.04% | Assay |
| IL-12 fusion polypeptide | | 30 | mins | ng/mL) | 12-point dose | Cytokine |
| (Free) | | | | | titration from 100 | quantification |
| IL-12 fusion polypeptide | | | | | ng/mL for each | by TR-FRET: |
| complexed to alum | | | | | dose treatment | IFNγ |
| (Rotating) | | | | | group | Additional |
| IL-12 fusion polypeptide | | 6 | hours | | | supernatants |
| (Free) | | | | | | stored for |
| IL-12 fusion polypeptide | | | | | | optional |
| complexed to alum | | | | | | cytokine |
| (Rotating) | | | | | | analysis |
| 1. Syringe for | High | 6 | hours | | | |

| | |
|---|---|
| all steps | Medium |
| | Low |
| 2. Syringe to | High |
| remove from | Medium |
| dose vial only | Low |
| 3. Pipette for | High |
| all steps | Medium |
| | Low |

CD = cluster of differentiation;
IFNγ = interferon gamma;
NA = not applicable;
PBMC = peripheral blood mononuclear cell;
TR-FRET = time-resolved fluorescence energy transfer

The CellTiter-Glo® 2.0 Cell Viability Assay was used to determine the number of viable cells in culture by quantitating the amount of adenosine triphosphate (ATP) present. This was used as an indication of the presence of metabolically active cells.

### Results

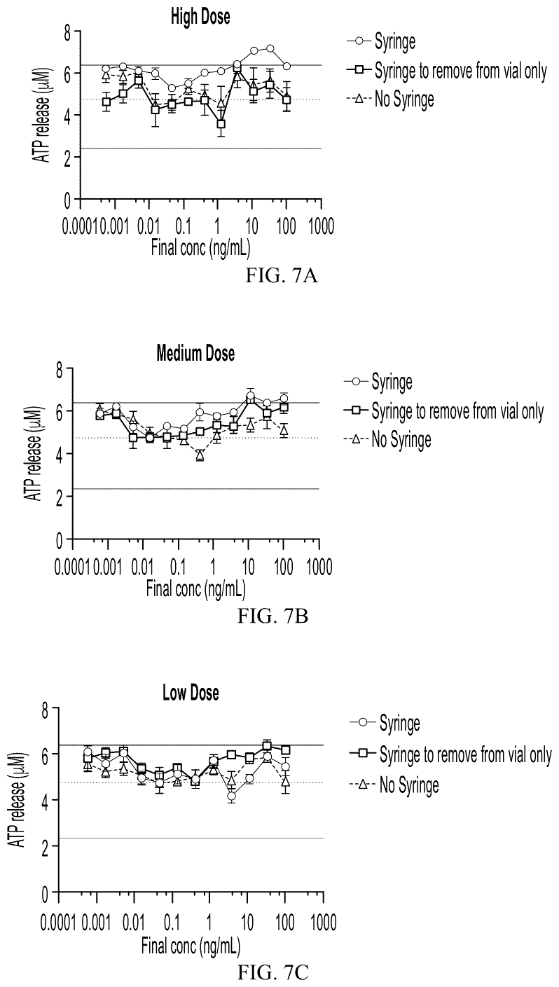

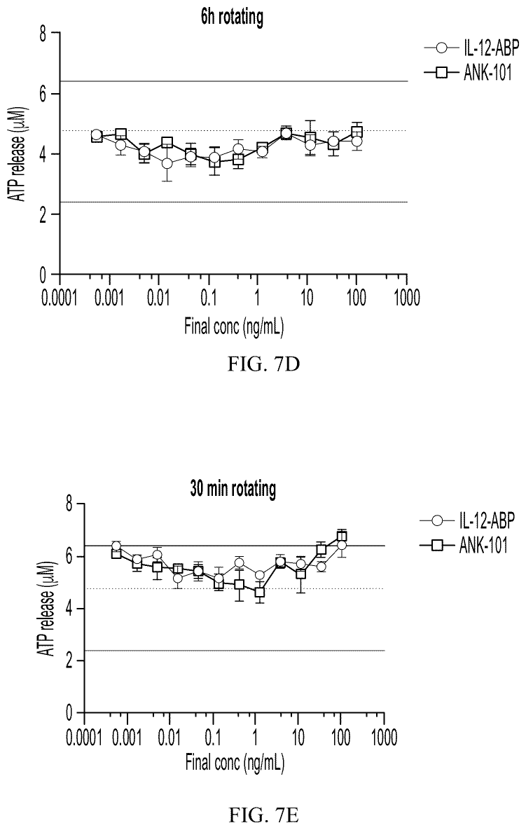

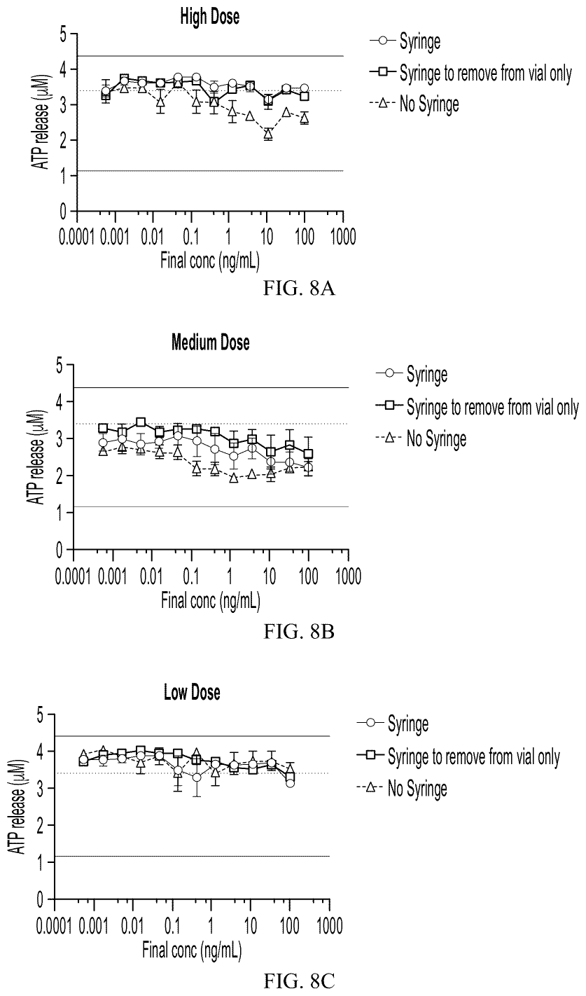

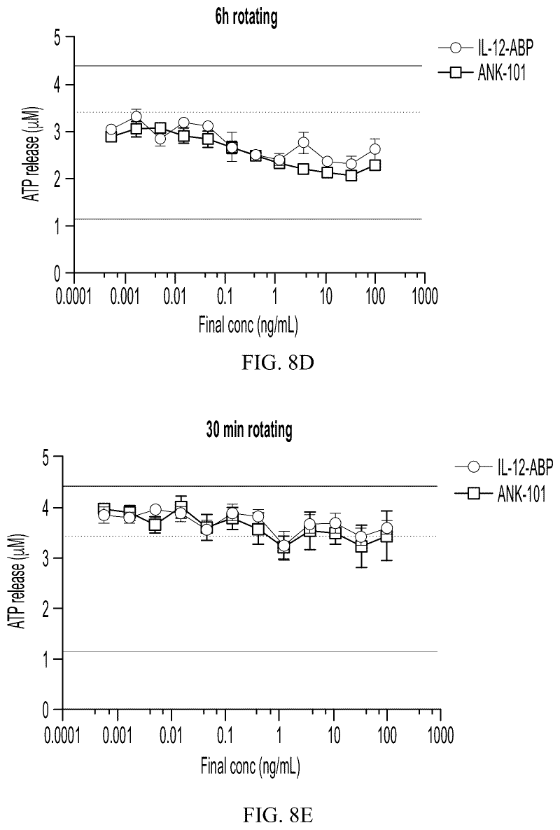

Across all 2 donors, stimulation with the positive control (anti-CD3 [5 μg/mL]+anti-CD28 [2 μg/mL]) increased levels of ATP above the unstimulated conditions (FIG. 7 and FIG. 8). In the presence of IL-12 fusion polypeptide complexed to alum, at all dose preparation groups, ATP levels were in line with the vehicle control for most protein concentrations, with a reduction in ATP levels seen at the top doses of IL-12 fusion polypeptide complexed to alum. Test compound preparation and incubation method did not appear to have a consistent impact on PBMC viability.

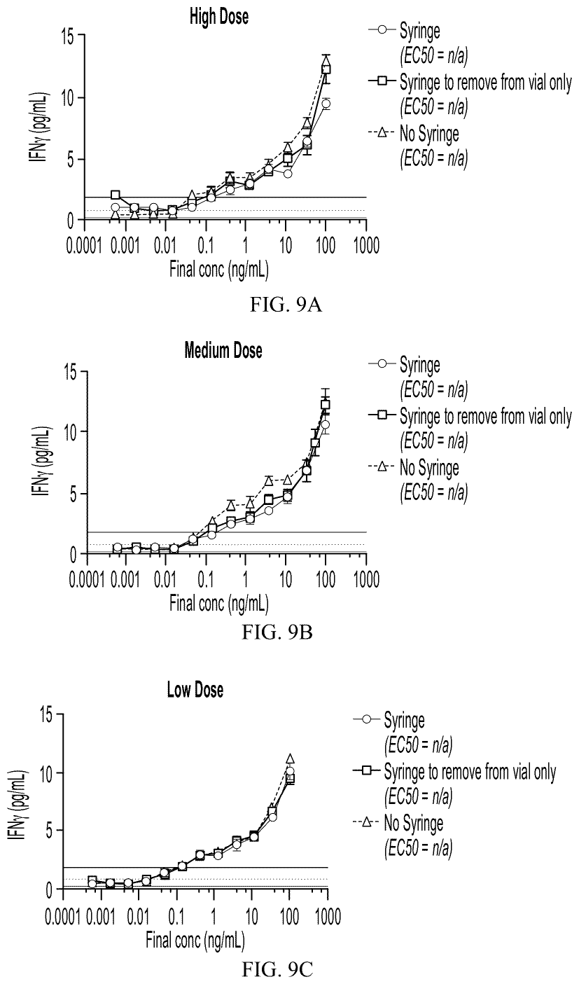

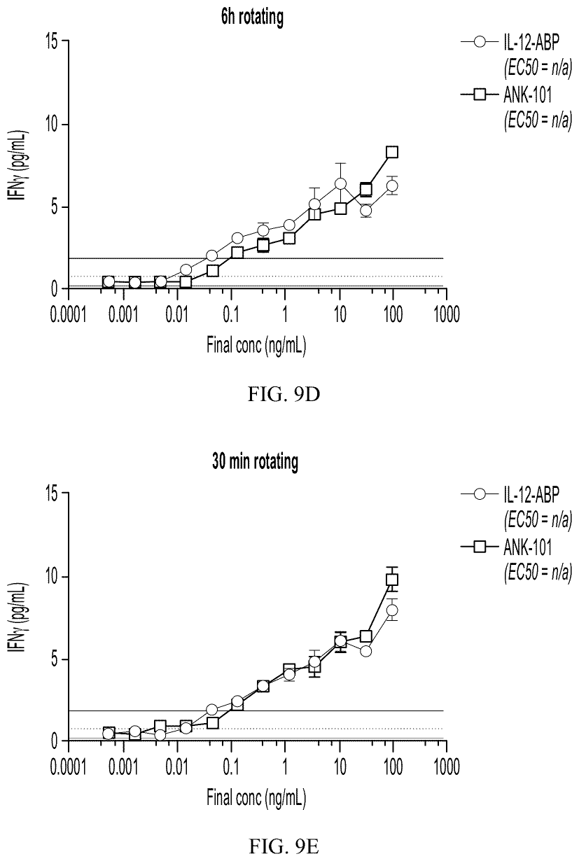

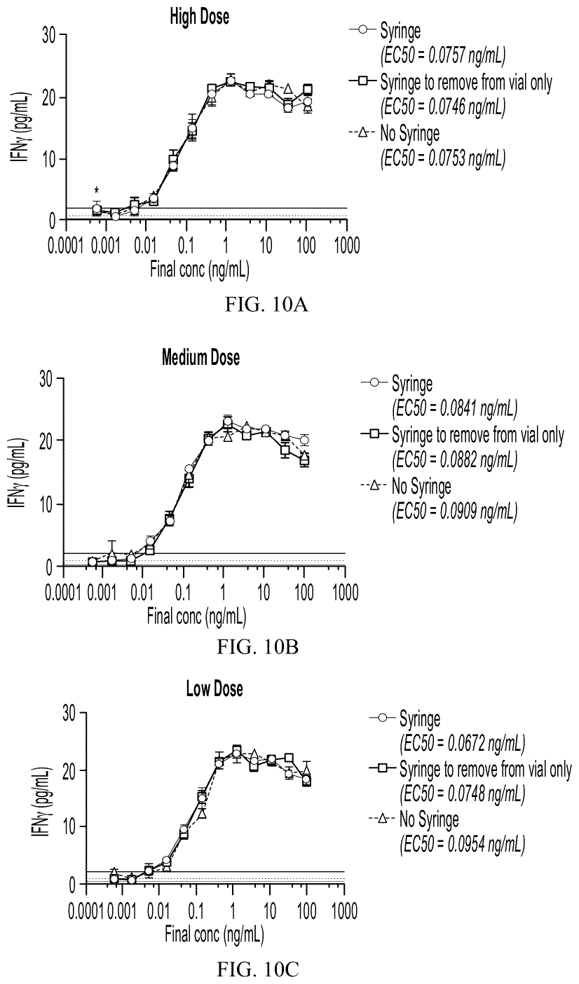

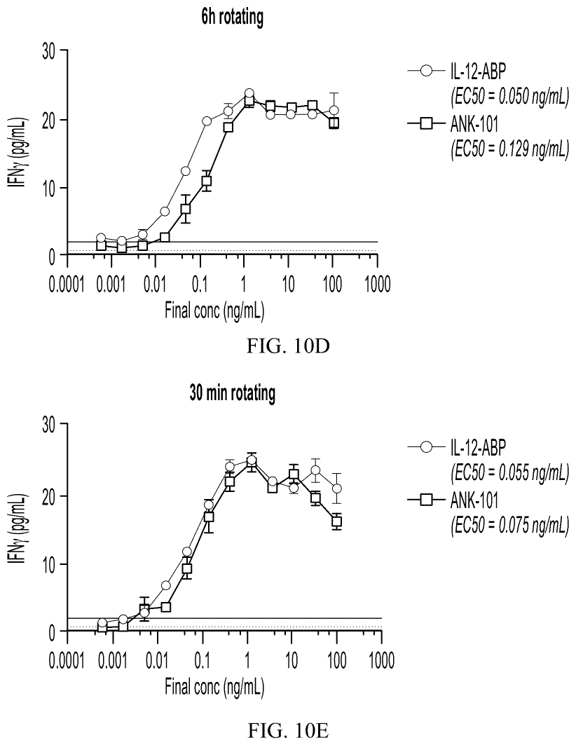

Stimulation with 100 ng/mL anti-CD3 induced sub-optimal stimulation of PBMC with moderate production of IFNγ. IL-12 fusion polypeptide complexed to alum at all dose preparation groups demonstrated enhanced IFNγ production in a dose-dependent manner (FIG. 9 and FIG. 10). There was limited difference in IFNγ production across both donors between the syringe and pipette preparation groups. The method of incubation (stationary vs. rotating) did not appear to impact IFNγ production. Increasing the incubation time caused a moderate reduction in the potency of IL-12 fusion polypeptide complexed to alum for the rotating condition only, however the half-maximal effective concentration (EC50) is still in line with non-rotating conditions at all 3 doses.

### Summary

IL-12 is an important T cell and NK cell stimulator and plays a vital role in driving the differentiation of T cells towards a pro-inflammatory phenotype by inducing production of IFNγ. To investigate the effect of preparation and administration method of IL-12 fusion polypeptide complexed to alum, EC₅₀ values were determined from the production of IFNγ by activated primary human PBMCs. The impact of the preparation method on PBMC viability was also determined. Three preparation methods (syringe, syringe to remove from dose vial only and no syringe [pipetting]) and 3 incubation periods (6 hours stationary, 6 hours with rotation and 30 minutes with rotation) were compared in this study-preparation with or without a syringe alongside a rotating set-up. The preparation conditions with or without a syringe were compared at 3 different dose groups (high, medium, or low).

At the end of the incubation period, in the absence of rotation, a high level of sedimentation of the complexes was noted. Each of the conditions were thoroughly inverted back and forth to ensure homogeneity.

Across both donors the cell viability, measured by ATP, of PBMCs remained consistent with a moderate reduction (Donor 2) in cell viability at the highest dose concentrations, comparable to previous studies observations. This reduction in cell viability was not consistently influenced by the preparation dose of IL-12 fusion polypeptide complexed to alum. Overall, preparation of IL-12 fusion polypeptide complexed to alum by syringe did not appear to have a consistent impact on PBMC viability.

Across both donors, IL-12 fusion polypeptide complexed to alum enhanced IFNγ production in a dose-dependent manner with a maximal response above the positive control (anti-CD3+anti-CD28). At all dose preparation groups, there was a limited difference in IFNγ production between the syringe and pipette preparation groups. Method of dose preparation did not appear to have an impact on the maximal response of cells, with a similar response across all doses whether prepared by syringe or pipette. EC₅₀ values were calculated only for Donor 2 as Donor 1 did not reach the maximal response. Increasing the incubation time may cause a decrease of the potency for IL-12 fusion polypeptide complexed to alum for the rotating condition only, however the EC₅₀ is still in line with non-rotating conditions at all 3 doses. Potency remained consistent across dose groups in Donor 2 ranging from 0.0541 ng/mL to 0.129 ng/mL, in line with historical IL-12 fusion polypeptide complexed to alum data.

Overall, the data suggests that method of dose preparation (syringe/no-syringe), incubation method (stationary/rotation), and length of incubation (30 minutes vs. 6 hours) did not have a significant impact on IL-12 fusion polypeptide complexed to alum potency or PBMC viability.

### Micro-BCA Assay of Complexed IL-12 Fusion Polypeptide.

The content (protein concentration) of the complexed IL-12 fusion polypeptide was interrogated with an in-use compatibility study that emulated drug preparation at the clinical pharmacy. The study utilized IL-12 fusion polypeptide drug substance (Lot P4130826), Alhydrogel® (Lot 152-001-001), Diluent (Lot 152-002-001), and SEVs, and commonly available ancillary components (e.g., syringes, needles). A bracketing design was employed to cover the intended dose range including an intermediate dose. A micro-BCA assay (Micro-BCA protein Assay Kit, Thermo Scientific) was employed to measure the quantity of IL-12 fusion polypeptide at the 3 different doses.

Dose group 6 (250 pg/mL), dose group 3 (20 pg/mL), and dose group 1 (2 pg/mL) samples were prepared. One set of dose controls was also prepared where all preparation was done by pipetting.

A micro-BCA assay was employed to quantify IL-12 fusion polypeptide bound to Alhydrogel® during an in-use bracketing study. The study included the highest potential dose to patients, as well as middle and lowest doses. Improvements in sample handling have enabled a more accurate quantitation of the protein in the high and middle doses while the low dose remains a challenge. The measured concentration of dosing preparations (same material and techniques such as these described in the Pharmacy Manual), did not vary appreciably to the dosing controls where the groups were prepared by pipetting. It appears that the use of syringes did not result in significant loss of protein during preparations. Precision in all cases is good, indicating that liquid handling by syringe is accurate enough to ensure reproducible dose dispensing at all volumes.

### Example 9: Stability Data Bulk IL-12 Fusion Polypeptide Drug Substance

The present Example demonstrates high stability of IL-12 fusion polypeptide drug substance reference standard batch and of IL-12 fusion polypeptide drug substance GMO batch.

Two IL-12 fusion polypeptide drug substances batches were tested:
500 L scale bioreactor batch (analytical reference standard batch) (P4130826ARS); and
1000 L scale bioreactor batch (Good Manufacturing Practice (GMP) batch) (1205114).

Summary of IL-12 Drug Substance stability is shown in Table 17.

TABLE 17

| | | | Stability | | Available | |
|---|---|---|---|---|---|---|
| Batch # | DoM | Manufacturer | Condition | | Data | |
| P4130826 | Aug. 3, 2022 | Lonza | −70° | C.¹ | 9 | Months |
| (Current RS lot) | | | 5° | C.² | | |
| | | | 25° | C.³ | | |
| 1205114 | May 11, 2023 | Lonza | −70° | C.¹ | 1 | Month |

(GMP Clinical
lot)

The stability protocol for IL-12 fusion polypeptide reference standard batch is shown in Table 18.

TABLE 18
Stability Protocol for IL-12 fusion polypeptide
Drug Substance Lot P4130826¹.

| | Timepoints (Months) | | | | | | | |
|---|---|---|---|---|---|---|---|---|
| Storage Condition | 0 | 1 | 2 | 3 | 6 | 9 | 12 | 18 |
| −70° C. (HDPE, Bottles) | NA | X | X | X | X | X | X | X |
| −70° C. (LDPE, Bag) | NA | X | X | X | X | X | X | X |
| +5° C. (HDPE, Bottles) | BR | X | X | X | X | X | X | X |
| +25° C. (HDPE, Bottles) | NA | X | X | X | X | NT | NT | NT |
| +5° C. (HDPE, Bottles, | B | NT | NT | NT | NT | NT | NT | NT |
| Bioburden) | | | | | | | | |
| +5° C. (LDPE Bag, Bioburden) | B* | NT | NT | NT | NT | NT | NT | NT |

¹Residual host cell deoxyribonucleic acid (DNA) and host cell protein (HCP) is not tested in the stability program; Bioburden is tested at T = 0 timepoint only; Malachite Green is tested for T = 0 only; anion exchange high-performance liquid chromatography (AEX-HPLC) is tested for information only;
Endotoxin is tested at T = 0 timepoint only
X = Clarity, color, protein concentration (content, pH, reduced reversed phase HPLC (RP-HPLC), size exclusion HPLC (SE HPLC), reduced capillary SDS, anion exchange HPLC (AEX-HPLC), activity
B = Six containers (two from the start, two from the middle and two from the end of the aliquoting process) removed from storage and transferred to QC Microbiology specifically for Bioburden testing
B* = Three bags (one from the start, one from the middle and one from the end of the aliquoting process) removed from storage and transferred to QC Microbiology specifically for Bioburden testing
BR = Batch release testing will be reported for T = 0 timepoint for Bioburden and Osmolality
HDPE = high density polyethylene;
LDPE = low density polyethylene;
NA = not applicable;
NT = not tested

The stability protocol for IL-12 fusion polypeptide GMO batch is shown in Table 19.

TABLE 19
Stability Protocol for IL-12 fusion polypeptide
Drug Substance Lot 1205114¹

| | Timepoints (Months) | | | | | | | | |
|---|---|---|---|---|---|---|---|---|---|
| Storage Condition | 0 | 1 | 2 | 3 | 6 | 9 | 12 | 18 | 24 |
| −70° C. (LDPE Bag) | NA | X | X | X | X | X | X | X | X |
| +5° C. (LDPE Bag) | BR | X | X | X | X | X | X | NT | NT |
| +25° C. (LDPE Bag) | NA | X | X | X | X | NT | NT | NT | NT |
| +5° C. (LDPE Bag, | B* | NT | NT | NT | NT | NT | NT | NT | NT |
| Bioburden) | | | | | | | | | |

¹Residual host cell DNA and HCP is not tested in the stability program; Bioburden is tested at T = 0 timepoint only; Malachite Green is tested for T = 0 only; AEX-HPLC is tested for information only;
Endotoxin is tested at T = 0 timepoint only
X = Clarity, color, protein concentration (content, pH, reduced reversed phase HPLC (RP-HPLC), size exclusion HPLC (SE HPLC), reduced capillary SDS, anion exchange HPLC (AEX-HPLC), activity
B* = Three bags (one from the start, one from the middle and one from the end of the aliquoting process) removed from storage and transferred to QC Microbiology specifically for Bioburden testing
BR = Batch release testing will be reported for T = 0 timepoint except for Bioburden and Osmolality
LDPE = low density polyethylene;
NA = not applicable;
NT = not tested

### Analytical Methods

An exemplary stability test method is described in this section. AEX-HPLC method was used for the separation of the dephosphorylated IL-12 fusion polypeptide drug substance and its charged variants. An AEX-HPLC column was used to quantify charged variants present in the LI-12 fusion polypeptide drug substance. The sample was dephosphorylated using phosphatase and injected onto the column. Charged variants were separated based on differences on the surface charge of different molecular species. Acidic molecules having negative charge elute later than basic molecules with positive charge. For IL-12 fusion polypeptide drug substance, the charged species can be based on differences in their surface charge and determined through detection of eluted peaks by fluorescence detection using excitation wavelength of 280 nm and emission wavelength of 320 nm. Relative quantification of the IL-12 fusion polypeptide drug substance acidic and basic species is achieved by relative area % evaluation.

### Summary

To-date, 1 month of stability data at each storage condition have been collected for IL-12 fusion polypeptide drug substance GMP batch and 9 months of stability at each storage condition for IL-12 fusion polypeptide drug substance analytical reference standard batch have been collected. All stability storage conditions (−70° C., +5° C. and +25° C.) met the stability study specification. All samples tested to date met the current GMP release specification. Drug substance analytical reference standard batch samples stored at +5° C. showed an apparent slight decrease in percentage main peak by reduced capillary electrophoresis sodium dodecyl sulfate (CE SDS) analysis at nine months compared to the study start, however this was within the expected intermediate precision percentage CV for the main peak (1% CV for reduced CE SDS).

Comparison of samples stored in low density polyethylene (LDPE) Bags and high-density polyethylene (HDPE) Bottles at −70° C. for nine months for IL-12 fusion polypeptide from the analytical reference standard batch showed consistent results, confirming that the container type had no marked effect on the product by any test methods.

Only during the stress condition of 25° C. and at the 6-month timepoint (analytical reference standard batch), there is a decrease in % main peak as evidenced by CE SDS with drug substance analytical reference standard batch.

A similar slight decrease at all conditions at the 1-month timepoint for GMP batch as evidenced by AEX-HPLC may be attributed to assay variability which will be interrogated during the planned assay validation.

### Example 10: Stability Data IL-12 Fusion Polypeptide Drug Product

The present Example demonstrates high stability of IL-12 fusion polypeptide drug product (reference standard batch and GMO batch).

A summary of the IL-12 fusion polypeptide drug product lots placed on stability is provided in Table 20. Stability indicating tests and associated conditions are listed below in Table 21 and Table 22.

TABLE 20
Summary of IL12-fusion polypeptide stability studies

| | | | Available |
|---|---|---|---|
| Lot # | Manufacturer | Stability Condition | Data |
| 101 | Lonza AG | −20° C. ± 5° C. (UP) | 9 Months |
| (Current RS, | Drug Product Services | −20° C. ± 5° C.* (UP) | |
| non-GMP Lot) | Hochbergerstrasse 60A 4057 | Endotoxin/ | |
| | Basel | bioburden | |
| | Switzerland | 5° C. ± 3° C. (UP) | |
| | | 5° C. ± 3° C. (UP)* | |
| | | Endotoxin/ | |
| | | bioburden | |
| | | 5° C. ± 3° C. (INV) | |
| | | 25° C. ± 2° C./60% | |
| | | RH ± 5% RH (INV) | |
| | | 40° C. ± 2° C./75% | |
| | | RH ± 5% RH (INV) | |
| 0001200579 | Lonza AG | −20° C. ± 5° C. (UP) | 0 |
| (GMP lot) | Standort Sterilproduktion Stein | 5° C. ± 3° C. (INV) | |
| | Schaffhauserstrasse 101 | 25° C. ± 2° C./60% | |
| | 4332 Stein | RH ± 5% RH (INV) | |
| | Switzerland | 40° C. ± 2° C./75% | |
| | | RH ± 5% RH (INV) | |

TABLE 21
Stability Protocol for IL-12 fusion
polypeptide Drug Product Lot 101

| | Time Points (months) | | | | | | | | |
|---|---|---|---|---|---|---|---|---|---|
| Storage Conditions | 0^{BR} | 1 | 3 | 6 | 9 | 12 | 18 | 24 | 36 |
| −20° C. ± 5° C. (UP) | BR | X | X | X | X | X | X | X | Opt. |
| 5° C. ± 3° C. (UP) | BR | NT | NT | X | NT | ◯ | NT | ◯ | Opt. |
| 5° C. ± 3° C. (INV) | BR | X | X | X | X | X | X | X | Opt. |
| 25° C. ± 2° C./ | BR | X | X | X | NT | NT | NT | NT | NT |
| 60% RH ± 5% RH (INV) | | | | | | | | | |
| 40° C. ± 2° C./ | BR | X | X | NT | NT | NT | NT | NT | NT |
| 75% RH ± 5% RH (INV) | | | | | | | | | |

BR = batch release;
INV = inverted, vials stored heads down;
NT = not tested;
Opt. = optional;
RH = relative humidity;
UP = upright, vials stored heads up;
X = color, clarity, pH, protein concentration (content), size exclusion-high-performance liquid chromatography (SE HPLC), capillary electrophoresis sodium dodecyl sulfate (CE SDS), reversed-phase high-performance liquid chromatography (RP HPLC), anion exchange high-performance liquid chromatography (AEX HPLC), surfactant content, activity, visible and sub-visible particles
◯ = bioburden only;
Note:
Batch release testing includes color, clarity, pH, osmolality, extractable volume, visible and sub-visible particles, protein concentration (content), size exclusion HPLC, activity, endotoxin, bioburden, identity by reduced reversed phase HPLC, purity by reduced by reversed phase HPLC, capillary electrophoresis SDS reduced, anion exchange HPLC; Extractable volume, osmolality, identity, endotoxin, tested at BR only; Surfactant content is tested from 1 M onwards for all conditions; Bioburden testing is done
for 5° C. ± 3° C. (UP) and −20° C. ± 5° C. (UP) at BR and annually thereafter

TABLE 22
Stability Protocol for IL-12 fusion
polypeptide Drug Product Lot 101

| | | Time Points (months) | | | | | | | |
|---|---|---|---|---|---|---|---|---|---|
| Storage Conditions | 0^{BR} | 1 | 3 | 6 | 9 | 12 | 18 | 24 | 36 |
| −20° C. ± | BR | X^{1, 2} | X | X | X | X | X | X | Opt. |
| 5° C. (UP) | | | | | | | | | |
| 5° C. ± | BR | NT | NT | ◯ | NT | ◯ | NT | ◯ | Opt. |
| 3° C. (UP) | | | | | | | | | |
| 5° C. ± | BR | X¹ | X | X | X | X | X | X | Opt. |
| 3° C. (INV) | | | | | | | | | |
| 25° C. ± | BR | X¹ | X | X | NT | NT | NT | NT | NT |
| 2° C./60% | | | | | | | | | |
| RH ± 5% RH (INV) | | | | | | | | | |
| 40° C. ± | BR | X | X | NT | NT | NT | NT | NT | NT |
| 2° C./75% | | | | | | | | | |
| RH ± 5% RH (INV) | | | | | | | | | |

BR = batch release;
INV = inverted, vials stored heads down;
NT = not tested;
Opt. = optional;
RH = relative humidity;
UP = upright, vials stored heads up;
◯ = color, clarity, pH, content, SE HPLC, CE SDS, RP HPLC, surfactant content, activity, visible and sub-visible particles;
X = color, clarity, pH, content, SE HPLC, CE SDS, RP HPLC, surfactant content, activity, visible and sub-visible particles, CCIT, osmolality, malachite green, LC MS (intact mass).
¹The 1 M −20° C. ± 5° C. (UP), 5° C. ± 3° C. (INV), and 25° C. ± 2° C./60% RH ± 5% RH (INV) conditions are not tested with LC MS (intact mass).
²The 1 M −20° C. ± 5° C. (UP) condition is not tested with malachite green.
Note:
Batch release testing includes color, clarity, pH, osmolality, extractable volume, visible and sub-visible particles, protein concentration (content), SE HPLC, activity, endotoxin, sterility, identity by RP-HPLC red., purity by reduced by RP-HPLC, CE-SDS red., surfactant content, AEX HPLC; Extractable volume, identity, endotoxin, sterility is tested at BR only; For stability, container closure integrity (CCIT) is tested for 5° C. ± 3° C. (INV) only, starting with 12 M and annually thereafter. Osmolality is tested for the 5° C. ± 3° C. (INV) and the 25° C. ± 2° C./60% RH ± 5% RH (INV) conditions, starting with the 3 M timepoint for both. Malachite green, AEX HPLC, and LC MS (intact mass) are employed for characterization purposes

### Exemplary Analytical Methods

AEX-HPLC method was used for the separation of the dephosphorylated IL-12 fusion polypeptide drug product and its charged variants. An AEX-HPLC column was used to quantify charged variants present in the IL-12 fusion polypeptide drug substance. The sample was dephosphorylated using phosphatase and injected onto the column. Charged variants were separated based on differences on the surface charge of different molecular species. Acidic molecules having negative charge elute later than basic molecules with positive charge. For IL-12 fusion polypeptide drug substance, the charged species can be based on differences in their surface charge and determined through detection of eluted peaks by fluorescence detection using excitation wavelength of 280 nm and emission wavelength of 320 nm. Relative quantification of the IL-12 fusion polypeptide drug product acidic and basic species is achieved by relative area % evaluation.

The Container Closure Integrity (CCI) test was performed using Helium Leak Test. The quantitative mass spectrometry-based helium leak physical container closure integrity method was used to test microbiological tightness of container closure systems. Container closure systems are placed into an airtight flange connected to the mass spectrometer, a vacuum pump creates a pressure difference between the inside of the mass spectrometer and the inside of the container closure system where helium gas was constantly applied into the container closure systems. The mass spectrometry instrument quantifies the helium gas flow in mbar L/s leaking through a potential leak in the container closure system.

### Conclusion

IL-12 fusion polypeptide drug product non-GMP lot, remained stable and met all release specifications at the 9-month timepoint and intended storage temperature of 5° C.±3° C. There was a minimal increase in 2:2 m subvisible particles for both upright and inverted vials. A similar profile was obtained when the IL-12 fusion polypeptide drug product was stored frozen at −20° C.±5° C. At the accelerated temperature of 25° C.±2° C./60%±5% relative humidity (RH) (inverted), and 6-month timepoint, IL-12 fusion polypeptide drug product, demonstrated slight decrease in main peak when tested with reduced reversed-phase high performance liquid chromatography (RP-HPLC) and reduced capillary electrophoresis sodium dodecyl sulfate (CE-SDS), compared to the previous timepoints. The molecule exhibited little, if any, tendency to aggregate (size exclusion-high-performance liquid chromatography [SE-HPLC]) or form subvisible particles. Only at the stress condition of 40° C.±2° C./75%±5% RH (inverted) and at the 3-month timepoints, IL-12 fusion polypeptide drug product exhibited a significant decrease in main peak (RP-HPLC, CE-SDS). At the same condition, IL-12 fusion polypeptide drug product also showed a slight decrease in % monomer when assayed by SE-HPLC. The IL-12 fusion polypeptide drug product GMP lot met all specifications at the time of lot release (0-month timepoint) and is comparable to IL-12 fusion polypeptide drug product non-GMP lot (0-month timepoints). It is expected that IL-12 fusion polypeptide drug product GMP lot will exhibit a similar stability profile to IL-12 fusion polypeptide drug product non-GMP lot.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A composition comprising
i) a phosphorylated form of a fusion polypeptide comprising:
a) an immunomodulatory polypeptide comprising an interleukin-12 immune agonist moiety; and
b) a metal hydroxide-binding polypeptide,
whose amino acid sequence includes a plurality of phosphorylation sites, so that the fusion polypeptide can adopt phosphorylated and unphosphorylated forms;
ii) Tris buffer;
iii) salt;
iv) sucrose;
v) L-Methonine; and
vi) a surfactant,
wherein the pH of the composition is within the range of about 6.5 and about 8.

2. The composition according to any of the preceding claims, wherein the fusion polypeptide, when exposed to a metal-hydroxide forms a complex therewith.

3. The composition according to any of the preceding claims, wherein the metal hydroxide is an aluminum hydroxide.

4. The composition according to any of the preceding claims, wherein the pH is within the range about 6.5 and about 7.8.

5. The composition according to any of the preceding claims, wherein, wherein the pH is within the range about 7 and about 7.6.

5. The composition according to any of the preceding claims, wherein the pH is about 7.3 to about 7.4.

6. The composition according to any of the preceding claims, wherein the concentration of the fusion polypeptide is within the range of about 0.1 g/L and about 15 g/L.

7. The composition according to any of the preceding claims, wherein the concentration of fusion polypeptide is within the range of about 0.5 g/L and about 5 g/L.

8. The composition according to any of the preceding claims, wherein the concentration of fusion polypeptide is within the range of about 1 g/L and about 3 g/L.

9. The composition according to any of the preceding claims, wherein the concentration of fusion polypeptide about 2 g/L.

10. The composition according to any of the preceding claims, wherein the concentration of Tris buffer is within the range of about 1 mM and about 50 mM.

11. The composition according to any of the preceding claims, wherein the concentration of Tris buffer is within the range of about 10 mM and about 25 mM.

12. The composition according to any of the preceding claims, wherein the concentration of salt is within the range of about 1 mM and about 750 mM.

13. The composition according to any of the preceding claims, wherein the concentration of salt is within the range of about 10 mM and about 100 mM.

14. The composition according to any of the preceding claims wherein the salt is NaCl or Na2SO4.

15. The composition according to any of the preceding claims, wherein the concentration of L-Methonine is within the range of about 1 mM and about 20 mM.

16. The composition according to any of the preceding claims, wherein the concentration of L-Methonine is within the range of about 5 mM and about 15 mM.

17. The composition according to any of the preceding claims wherein the concentration of L-Methonine is 10 mM.

18. The composition according to any of the preceding claims, wherein the surfactant is a polysorbate.

19. The composition according to any of the preceding claims, wherein the surfactant is a polysorbate 20 or polysorbate 80.

20. The composition according to any of the preceding claims, wherein the surfactant is a polysorbate 20.

21. The composition according to any of the preceding claims, wherein the concentration of polysorbate is within the range of about 0.005% w/v and about 0.1% w/v.

22. The composition according to any of the preceding claims, wherein the concentration of polysorbate is within the range of about 0.01% w/v and about 0.05% w/v.

23. The composition according to any of the preceding claims, wherein the concentration of polysorbate is about 0.02% w/v.

24. The composition according to any of the preceding claims, wherein the concentration of sucrose is within the range of about 100 mM and about 200 mM.

25. The composition according to any of the preceding claims, wherein the concentration of sucrose is 150 mM.

26. The composition according to any of the preceding claims, wherein the composition comprises 2 mg/mL fusion polypeptide, 20 mM Tris buffer, 50 mM NaCl, 10 mM L-Methonine, 0.02% polysorbate 20, and 150 mM sucrose, and wherein the pH of the composition is within the range of 6 and 8.

27. The composition according to any of the preceding claims, wherein the composition has been stored at a temperate at the most −50° C., such as at the most −55° C., such as at the most −60° C., such as at the most −65° C.

28. The composition according to any of the preceding claims, wherein the composition has been stored within the range of about 1 day to about 500 days.

29. The composition according to any of the preceding claims wherein the pH of the composition after storage is comparable to the pH of the composition prior to storage.

30. The composition according to any of the preceding claims, wherein the concentration of the fusion polypeptide in the composition after storage is comparable to the concentration of the fusion polypeptide in the composition prior to storage.

31. The composition according to any of the preceding claims, wherein the osmolarity of the composition after storage is comparable to the osmolarity of the composition prior to storage.

32. The composition according to any of the preceding claims, wherein the color of the composition after storage is comparable to the color of the composition prior to storage.

33. The composition according to any of the preceding claims, wherein the amount of visible particles in the composition is comparable to the amount of visible particles of the composition prior to storage.

34. The composition according to any of the preceding claims, wherein the formulation is a liquid composition.

35. The composition according to any of the preceding claims, wherein the formulation is a solid composition.

36. The composition according to any of the preceding claims, wherein the composition is in a dry form.

37. The composition according to any of the preceding claims, wherein the formulation is a powder.

38. The composition according to any of the preceding claims, wherein the composition is frozen.

39. The composition according to any of the preceding claims, wherein the composition is in a vial.

40. The composition according to any of the preceding claims, wherein the vial is light protected.

41. A pharmaceutical formulation comprising
i) a fusion polypeptide metal-hydroxide complex comprising:
(a) a phosphorylated form of a fusion polypeptide comprising:
ai) an immunomodulatory polypeptide comprising an interleukin-12 immune agonist moiety; and
aii) a metal hydroxide-binding polypeptide
wherein the fusion polypeptide metal-hydroxide complex amino acid sequence includes a plurality of phosphorylation sites, so that it can adopt phosphorylated and unphosphorylated forms; and
(b) a metal hydroxide
ii) Tris buffer;
iii) salt;
iv) sucrose;
v) L-Methonine; and
vi) a surfactant,
wherein the pH of the composition is within the range of about 6.5 and about 8.

42. The pharmaceutical formulation of claim 41, wherein the fusion polypeptide is adsorbed via ligand exchange to the metal hydroxide via the at least one phosphorylated amino acid of the metal hydroxide-binding peptide, thereby forming fusion polypeptide metal-hydroxide complex.

43. The pharmaceutical formulation of claims 41-42, wherein the metal hydroxide is an aluminum hydroxide.

44. The pharmaceutical formulation of claims 41-43, wherein the concentration of the aluminum hydroxide is within the range of about 0.5 mg/mL and about 10 mg/mL.

45. The pharmaceutical formulation of claims 41-44, wherein the concentration of the aluminum hydroxide is within the range of about 1 mg/mL and about 5 mg/mL.

46. The pharmaceutical formulation of claims 41-45, wherein the concentration of the aluminum hydroxide is 2.5 mg/mL.

47. The pharmaceutical formulation of claims 41-46, wherein the pH is within the range about 6.8 and about 7.8.

48. The pharmaceutical formulation of claims 41-47, wherein the pH is within the range about 7 and about 7.6.

49. The pharmaceutical formulation of claims 41-48, wherein the pH is about 7.3.

50. The pharmaceutical formulation of claims 41-49, wherein the concentration of the fusion polypeptide is within the range of about 0.0025 mg/mL and about 1 mg/mL.

51. The pharmaceutical formulation of claims 41-50, wherein the concentration of the fusion polypeptide is within the range of about 0.05 mg/mL and about 0.75 mg/mL.

52. The pharmaceutical formulation of claims 41-51, wherein the concentration of fusion polypeptide is within the range of about 0.1 mg/mL and about 0.5 mg/mL.

53. The pharmaceutical formulation of claims 41-52, wherein the concentration of the fusion polypeptide is 0.25 mg/mL.

54. The pharmaceutical formulation of claims 41-53, wherein the concentration of Tris buffer is within the range of about 1 mM and about 50 mM.

55. The pharmaceutical formulation of claims 41-54, wherein the concentration of Tris buffer is within the range of about 10 mM and about 40 mM.

56. The pharmaceutical formulation of claims 41-55, wherein the concentration of Tris buffer is is within the range of about 15 mM and about 20 mM.

57. The pharmaceutical formulation of claims 41-56, wherein the concentration of the salt is within the range of about 1 mM and about 100 mM.

58. The pharmaceutical formulation of claims 41-57, wherein the concentration of the salt is within the range of about 20 mM and about 60 mM.

59. The pharmaceutical formulation of claims 41-58, wherein the concentration of the salt is within the range of about 38 mM and about 50 mM.

60. The pharmaceutical formulation of claims 41-59, wherein the salt is NaCl or Na2SO4.

61. The pharmaceutical formulation of claims 41-60, wherein the concentration of L-Methonine is within the range of about 1 mM and about 20 mM.

62. The pharmaceutical formulation of claims 41-61, wherein the concentration of L-Methonine is within the range of about 5 mM and about 15 mM.

63. The pharmaceutical formulation of claims 41-62, wherein the concentration of L-Methonine is within the range of about 7.5 mM and about 10 mM.

64. The pharmaceutical formulation of claims 41-63, wherein the surfactant is a polysorbate.

65. The pharmaceutical formulation of claims 41-64, wherein the surfactant is a polysorbate 20 or polysorbate 80.

66. The pharmaceutical formulation of claims 41-65, wherein the surfactant is a polysorbate 20.

67. The pharmaceutical formulation of claims 41-66, wherein the concentration of polysorbate is within the range of about 0.005% w/v and about 0.1% w/v.

68. The pharmaceutical formulation of claims 41-67, wherein the concentration of polysorbate is within the range of about 0.01% w/v and about 0.05% w/v.

69. The pharmaceutical formulation of claims 41-68, wherein the concentration of polysorbate is about 0.015% w/v.

70. The pharmaceutical formulation of claims 41-69, wherein the concentration of sucrose is within the range of about 100 mM and about 200 mM.

71. The pharmaceutical formulation of claims 41-70, wherein the concentration of sucrose is 113 mM.

72. The pharmaceutical formulation of claims 41-71, wherein the formulation is a liquid composition.

73. The pharmaceutical formulation of claims 41-72, wherein the formulation is a solid composition.

74. The pharmaceutical formulation of claims 41-73, wherein the formulation is a powder.

75. The pharmaceutical formulation of claims 41-74, wherein the composition comprises 0.25 mg/mL fusion polypeptide, 15 mM Tris buffer, 38 mM NaCl, 7.5 mM L-Methonine, 0.015% polysorbate 20, and 113 mM sucrose, 2.5 mg/mL aluminum hydroxide and wherein the pH of the composition is within the range of 6 and 8.

76. The pharmaceutical formulation of claims 41-75, wherein the formulation is a liquid formulation.

77. The pharmaceutical formulation of claims 41-76, therein the formulation is formulated for parenteral delivery.

78. The pharmaceutical formulation of claims 41-77, wherein the formulation is formulated for intra-tumoral injection.

79. The pharmaceutical formulation of claims 41-79, wherein the formulation is in a vial.

80. A method of treating a subject, comprising administering a pharmaceutical composition according to claim 41.

81. The method of claim 80, wherein the subject has cancer.

82. The method of claim 81, wherein the cancer is associated with a tumor.

83. The method of claim 80, wherein the subject is a human.

84. The method of claim 80, wherein the composition is administered by parenteral administration.

85. The method of claim 82, wherein the composition is administered by an intra-tumoral injection.

86. The method of claim 83, wherein the composition is administered by a peri-tumoral injection.

87. The method of claim 80, wherein the composition is administered in combination with a second therapy.

88. The method of claim 87, wherein the second therapy is a check point inhibitor.

89. A method of manufacturing a composition of claim 1, the method comprising combining the phosphorylated form of the fusion polypeptide with a Tris buffer, salt, sucrose, L-methionine and a surfactant.

90. A method of manufacturing a pharmaceutical formulation of claim 41, the method comprising:
i) contacting the composition of claim 1 with a metal-hydroxide.

91. The method of claim 90, wherein the contacting is performed for 1 minute to 60 minutes.

92. The method of claim 91, wherein the contacting is performed at room temperature.

93. A method of characterizing a composition of claim 1, by assessing degree of phosphorylation of the fusion polypeptide.

94. The method of claim 93, wherein the characterization includes assessing the purity of the fusion polypeptide in the composition.
